SEQUENCE LISTING (529 sequences)

SEQ ID NO: 1 - protein, 8 aa, Artificial Sequence
  Synthetic
GFTFSDYY

SEQ ID NO: 2 - protein, 8 aa, Artificial Sequence
  Synthetic
IDSSAGAI

SEQ ID NO: 3 - protein, 13 aa, Artificial Sequence
  Synthetic
ARERRDGHKIFDC

SEQ ID NO: 4 - protein, 12 aa, Artificial Sequence
  Synthetic
QNILYSSNNKNY

SEQ ID NO: 5 - protein, 3 aa, Artificial Sequence
  Synthetic
WSS

SEQ ID NO: 6 - protein, 9 aa, Artificial Sequence
  Synthetic
QQYYSSPYN

SEQ ID NO: 7 - protein, 120 aa, Artificial Sequence
  Synthetic
QVQLVESGGDLVKPGGSLRLSCVASGFTFSDYYMNWIRLPPGKGLEWVSYIDSSAGAIYYADSVRGRFTVSRDDAKNSLFLHMNSLRADDTAVYYCARERRDGHKIFDCWGPGTLVTVSS

SEQ ID NO: 8 - protein, 113 aa, Artificial Sequence
  Synthetic
DIVMTQSPDSLAVSLGERATINCKSSQNILYSSNNKNYLAWYQQKAGQPPKLLIFWSSTRESGVPDRFSGSGSGTDFTLTINSLQAEDVAVYYCQQYYSSPYNFGRGTTLEIK

SEQ ID NO: 9 - protein, 8 aa, Artificial Sequence
  Synthetic
GFTFSDYY

SEQ ID NO: 10 - protein, 8 aa, Artificial Sequence
  Synthetic
IDSSAGAI

SEQ ID NO: 11 - protein, 13 aa, Artificial Sequence
  Synthetic
ARERRDGHKIFDC

SEQ ID NO: 12 - protein, 12 aa, Artificial Sequence
  Synthetic
QNILYSSNNKNY

SEQ ID NO: 13 - protein, 3 aa, Artificial Sequence
  Synthetic
WSS

SEQ ID NO: 14 - protein, 9 aa, Artificial Sequence
  Synthetic
QQYYSSPYN

SEQ ID NO: 15 - protein, 120 aa, Artificial Sequence
  Synthetic
QVHLVESGGDLVKPGGSLRLSCVASGFTFSDYYMNWIRMPPGKGLEWISYIDSSAGAIYYADSVKGRFTVSRDDAKNSLFLHMNNLRADDTAVYYCARERRDGHKIFDCWGPGTLVTVSS

SEQ ID NO: 16 - protein, 113 aa, Artificial Sequence
  Synthetic
DIVMTQSPDSLAVSLGERATINCKSSQNILYSSNNKNYLAWYQQKAGQPPKLLIFWSSTRESGVPDRFSGSGSGTDFTLTISSLQAEDVAVYYCQQYYSSPYNFGRGTTLEIK

SEQ ID NO: 17 - protein, 8 aa, Artificial Sequence
  Synthetic
GYSFTYYW

SEQ ID NO: 18 - protein, 8 aa, Artificial Sequence
  Synthetic
IYPGDSDT

SEQ ID NO: 19 - protein, 12 aa, Artificial Sequence
  Synthetic
TTHARNWNNVAY

SEQ ID NO: 20 - protein, 6 aa, Artificial Sequence
  Synthetic
QNINTF

SEQ ID NO: 21 - protein, 3 aa, Artificial Sequence
  Synthetic
AAS

SEQ ID NO: 22 - protein, 9 aa, Artificial Sequence
  Synthetic
QQSSSTPWT

SEQ ID NO: 23 - protein, 119 aa, Artificial Sequence
  Synthetic
EVQLVQSGAEVKKPGESLKISCKGSGYSFTYYWIGWVRQMPGKGLEWMGIIYPGDSDTRYSPSFQGQVTISADKSISTAYLQWSSLKASDTAMYYCTTHARNWNNVAYWGQGTLVTVSS

SEQ ID NO: 24 - protein, 107 aa, Artificial Sequence
  Synthetic
DIQMTQSPSSLSASVGDRLTITCRASQNINTFLNWYQQNPGKAPKVLIYAASSLESGVPSRFSGSGSGTDFTLTISSLQPEDFATYYCQQSSSTPWTFGQGTKVEIK

SEQ ID NO: 25 - protein, 8 aa, Artificial Sequence
  Synthetic
GFTFSGFA

SEQ ID NO: 26 - protein, 8 aa, Artificial Sequence
  Synthetic
ISGSGDIT

SEQ ID NO: 27 - protein, 16 aa, Artificial Sequence
  Synthetic
AKDPYNWNHGVYGMDV

SEQ ID NO: 28 - protein, 6 aa, Artificial Sequence
  Synthetic
QSISSY

SEQ ID NO: 29 - protein, 3 aa, Artificial Sequence
  Synthetic
GAS

SEQ ID NO: 30 - protein, 9 aa, Artificial Sequence
  Synthetic
QQSFNAPLT

SEQ ID NO: 31 - protein, 123 aa, Artificial Sequence
  Synthetic
EVQLLESGGGLAQPGKSLRLSCAASGFTFSGFAMSWVRQAPGKGLEWVSGISGSGDITYYADSVKGRFTISRDNSKSTLYLQMNSLGAEDTAVYYCAKDPYNWNHGVYGMDVWGQGTTVTVSS

SEQ ID NO: 32 - protein, 107 aa, Artificial Sequence
  Synthetic
DIQMTQSPSSLSASVGDRVTITCRASQSISSYLNWYQQKPGKAPKFLIYGASSLQSGVPSRFSGSGSGTDFTLTISSLQPEDFAAYYCQQSFNAPLTFGGGTKVEIK

SEQ ID NO: 33 - protein, 8 aa, Artificial Sequence
  Synthetic
GFTFSDYY

SEQ ID NO: 34 - protein, 8 aa, Artificial Sequence
  Synthetic
IDSSAGAI

SEQ ID NO: 35 - protein, 13 aa, Artificial Sequence
  Synthetic
ARERRDGHKIFDW

SEQ ID NO: 36 - protein, 12 aa, Artificial Sequence
  Synthetic
QNILYTSNNKNY

SEQ ID NO: 37 - protein, 3 aa, Artificial Sequence
  Synthetic
WSS

SEQ ID NO: 38 - protein, 9 aa, Artificial Sequence
  Synthetic
HQYYSSPYT

SEQ ID NO: 39 - protein, 120 aa, Artificial Sequence
  Synthetic
QVQLVESGGGLVKPGGSLRLSCVASGFTFSDYYMNWIRLPPGRGLQWISYIDSSAGAIYYADSVKGRFTVSRDDAKNSLYLQMNSLRADDTAVYYCARERRDGHKIFDWWGQGTLVTVSS

SEQ ID NO: 40 - protein, 113 aa, Artificial Sequence
  Synthetic
DIVMTQSPDSLAVSLGERATINCKSSQNILYTSNNKNYLGWYQQKAGQPPKLLIYWSSTRNSGVPDRFSGSGSGTDFTLTISSLQAEDVAVYYCHQYYSSPYTFGQGTNLEIK

SEQ ID NO: 41 - protein, 8 aa, Artificial Sequence
  Synthetic
GYSFTNYW

SEQ ID NO: 42 - protein, 8 aa, Artificial Sequence
  Synthetic
IYPGDSDT

SEQ ID NO: 43 - protein, 12 aa, Artificial Sequence
  Synthetic
ATHARSWNYVAY

SEQ ID NO: 44 - protein, 6 aa, Artificial Sequence
  Synthetic
QSVNNF

SEQ ID NO: 45 - protein, 3 aa, Artificial Sequence
  Synthetic
AAS

SEQ ID NO: 46 - protein, 9 aa, Artificial Sequence
  Synthetic
QQSNTTPWT

SEQ ID NO: 47 - protein, 119 aa, Artificial Sequence
  Synthetic
EVQLVQSGAEVKKPGESLKISCKGSGYSFTNYWIGWVRQMPGKGLEWMGIIYPGDSDTRYSPSFQGQVTLSADKSISTAYLQWNSLKASDTAMYYCATHARSWNYVAYWGQGTLVTVAS

SEQ ID NO: 48 - protein, 107 aa, Artificial Sequence
  Synthetic
DIQMTQSPSSLSASVGDRVTITCRASQSVNNFLNWYQQKPGTAPKLLIYAASSLQGGVPSRFSGSGSGTDFTLTISSLQPEDFATYYCQQSNTTPWTFGQGTKVEIK

SEQ ID NO: 49 - protein, 8 aa, Artificial Sequence
  Synthetic
GFTFSDYY

SEQ ID NO: 50 - protein, 8 aa, Artificial Sequence
  Synthetic
IDSSAGAI

SEQ ID NO: 51 - protein, 13 aa, Artificial Sequence
  Synthetic
ARERRDGHKIFDC

SEQ ID NO: 52 - protein, 12 aa, Artificial Sequence
  Synthetic
QNILYSSNNKNY

SEQ ID NO: 53 - protein, 3 aa, Artificial Sequence
  Synthetic
WSS

SEQ ID NO: 54 - protein, 9 aa, Artificial Sequence
  Synthetic
QQYYSSPYN

SEQ ID NO: 55 - protein, 120 aa, Artificial Sequence
  Synthetic
QVQLVESGGDLVKPGGSLRLSCVASGFTFSDYYMNWIRLPPGKGLEWISYIDSSAGAIYYADSVKGRFTVSRDDAKNSLFLHMNSLRADDTAVYYCARERRDGHKIFDCWGPGTLVTVSS

SEQ ID NO: 56 - protein, 113 aa, Artificial Sequence
  Synthetic
DIVMTQSPDSLAVSLGERATINCKSSQNILYSSNNKNYLAWYQQKTGQPPKLLIFWSSTRESGVPDRFSGSGSGTDFTLTISSLQAEDVAVYYCQQYYSSPYNFGRGTTLEIK

SEQ ID NO: 57 - protein, 8 aa, Artificial Sequence
  Synthetic
GFTFDDYA

SEQ ID NO: 58 - protein, 8 aa, Artificial Sequence
  Synthetic
ISWSGVTM

SEQ ID NO: 59 - protein, 11 aa, Artificial Sequence
  Synthetic
ARGDGTNAFDI

SEQ ID NO: 60 - protein, 12 aa, Artificial Sequence
  Synthetic
QSVLYKSNNKNY

SEQ ID NO: 61 - protein, 3 aa, Artificial Sequence
  Synthetic
WAS

SEQ ID NO: 62 - protein, 9 aa, Artificial Sequence
  Synthetic
QQYYSVPLT

SEQ ID NO: 63 - protein, 118 aa, Artificial Sequence
  Synthetic
EVQLVESGGDLVQPGRSLRLSCAASGFTFDDYAMHWVRQGPGKGLEWVSGISWSGVTMGYADSVKGRFTISRDNAKNSLYLRMNSLRADDTAFYYCARGDGTNAFDIWGHGTMVTVSS

SEQ ID NO: 64 - protein, 113 aa, Artificial Sequence
  Synthetic
DIVMTQSPDSLAVSLGERATINCKSSQSVLYKSNNKNYLDWYQQKPGQPPKLLIYWASSRESGVPDRFSGSGSGTDFTLTISSLQAEDVAVYYCQQYYSVPLTFGGGTKVEIK

SEQ ID NO: 65 - protein, 8 aa, Artificial Sequence
  Synthetic
GFTFSDYY

SEQ ID NO: 66 - protein, 8 aa, Artificial Sequence
  Synthetic
IDSSAGAI

SEQ ID NO: 67 - protein, 13 aa, Artificial Sequence
  Synthetic
ARERRDGHKIFDC

SEQ ID NO: 68 - protein, 12 aa, Artificial Sequence
  Synthetic
QNILYSSNNKNY

SEQ ID NO: 69 - protein, 3 aa, Artificial Sequence
  Synthetic
WSS

SEQ ID NO: 70 - protein, 9 aa, Artificial Sequence
  Synthetic
QQYYSSPYN

SEQ ID NO: 71 - protein, 120 aa, Artificial Sequence
  Synthetic
QVQLVESGGDLVKPGGSLRLSCVASGFTFSDYYMNWIRLPPGKGLEWISYIDSSAGAIYYADSVKGRFTVSRDDAKNSLFLHMNNLRADDTAVYYCARERRDGHKIFDCWGPGTLVTVSS

SEQ ID NO: 72 - protein, 113 aa, Artificial Sequence; identical to SEQ ID NO: 16
  Synthetic

SEQ ID NO: 73 - protein, 8 aa, Artificial Sequence
  Synthetic
GFTFSDYY

SEQ ID NO: 74 - protein, 8 aa, Artificial Sequence
  Synthetic
IDSSAGAI

SEQ ID NO: 75 - protein, 13 aa, Artificial Sequence
  Synthetic
ARERRDGHKIFDY

SEQ ID NO: 76 - protein, 12 aa, Artificial Sequence
  Synthetic
QNILYSSNNKNY

SEQ ID NO: 77 - protein, 3 aa, Artificial Sequence
  Synthetic
WSS

SEQ ID NO: 78 - protein, 9 aa, Artificial Sequence
  Synthetic
HQYYSSPYN

SEQ ID NO: 79 - protein, 120 aa, Artificial Sequence
  Synthetic
QVQLVESGGDLVKPGGSLRLSCVASGFTFSDYYMNWIRLPPGRGLEWISYIDSSAGAIYYADSVKGRFTVSRDDAKKSLFLYMNSLRADDTAVYYCARERRDGHKIFDYWGPGTLVTVSS

SEQ ID NO: 80 - protein, 113 aa, Artificial Sequence
  Synthetic
DIVMTQSPDSLAVSLGERATINCKSSQNILYSSNNKNYLAWYQQKAGQPPKLLIFWSSTRESGVPDRFSGSGSGTDFTLTISSLQAEDVAVYYCHQYYSSPYNFGRGTTLEIK

SEQ ID NO: 81 - protein, 8 aa, Artificial Sequence
  Synthetic
GFTFSSFA

SEQ ID NO: 82 - protein, 8 aa, Artificial Sequence
  Synthetic
ISGSGGST

SEQ ID NO: 83 - protein, 16 aa, Artificial Sequence
  Synthetic
AKDKRNWNYGIDSFDF

SEQ ID NO: 84 - protein, 6 aa, Artificial Sequence
  Synthetic
QSISGY

SEQ ID NO: 85 - protein, 3 aa, Artificial Sequence
  Synthetic
ATS

SEQ ID NO: 86 - protein, 9 aa, Artificial Sequence
  Synthetic
HQSHSPPFT

SEQ ID NO: 87 - protein, 123 aa, Artificial Sequence
  Synthetic
EVQLLESGGGLVQPGGSLRLSCAASGFTFSSFAMSWVRQAPGKGLEWVSGISGSGGSTYYADSVKGRFTISRDNSKNTLYLQMNSLRAEDTAIYYCAKDKRNWNYGIDSFDFWGQGTMVTVSS

SEQ ID NO: 88 - protein, 107 aa, Artificial Sequence
  Synthetic
DIQMTQSPSSLSASVGGRVTITCRTSQSISGYLNWYQQKAGKAPKLLIYATSNLQSGVPSRFTGSGSGTDFTLTISSLQPEDFAAYYCHQSHSPPFTFGPGTKVAFK

SEQ ID NO: 89 - protein, 8 aa, Artificial Sequence
  Synthetic
GFTFSSYE

SEQ ID NO: 90 - protein, 8 aa, Artificial Sequence
  Synthetic
ISSGGSSI

SEQ ID NO: 91 - protein, 15 aa, Artificial Sequence
  Synthetic
ARVGGSYYYSYAMDV

SEQ ID NO: 92 - protein, 6 aa, Artificial Sequence
  Synthetic
QDISSY

SEQ ID NO: 93 - protein, 3 aa, Artificial Sequence
  Synthetic
AAS

SEQ ID NO: 94 - protein, 9 aa, Artificial Sequence
  Synthetic
QQPHNSPFS

SEQ ID NO: 95 - protein, 122 aa, Artificial Sequence
  Synthetic
EVQLVESGGGLVQPGGSLRLSCAASGFTFSSYEMNWVRQAPGKGLEWVSYISSGGSSIKYADSVRGRFAFSRDNAENSVHLQMNSLRAEDTAVYYCARVGGSYYYSYAMDVWGQGTAVTVSS

SEQ ID NO: 96 - protein, 107 aa, Artificial Sequence
  Synthetic
DIQLTQSPSFLSASVGDRVTITCWASQDISSYLAWYQQKPGKAPKLLIYAASTLQSGVPSRFTGSGSGTEFTLTISSLQPEDFATYYCQQPHNSPFSFGPGTKVDVK

SEQ ID NO: 97 - protein, 8 aa, Artificial Sequence
  Synthetic
GFTFSDYY

SEQ ID NO: 98 - protein, 8 aa, Artificial Sequence
  Synthetic
IDSSAGAI

SEQ ID NO: 99 - protein, 13 aa, Artificial Sequence
  Synthetic
VRERRDGHKIFDY

SEQ ID NO: 100 - protein, 12 aa, Artificial Sequence
  Synthetic
QNILYSSNNKNY

SEQ ID NO: 101 - protein, 3 aa, Artificial Sequence
  Synthetic
WSS

SEQ ID NO: 102 - protein, 9 aa, Artificial Sequence
  Synthetic
QQYYSSPYN

SEQ ID NO: 103 - protein, 120 aa, Artificial Sequence
  Synthetic
QVQLVESGGDLVKPGGSLRLSCVASGFTFSDYYMNWIRLPPGKGLEWISYIDSSAGAIYYADSVKGRFTVSRDDAKNSLFLHMNSLRADDTAVYYCVRERRDGHKIFDYWGPGTLVTVSS

SEQ ID NO: 104 - protein, 113 aa, Artificial Sequence
  Synthetic
DIVMTQSPDSLAVSLGERATINCKSSQNILYSSNNKNYLAWYQQKAGQPPKLLIFWSSIRESGVPDRFSGSGSGTDFTLTISSLQAEDVAVYYCQQYYSSPYNFGRGTTLEIK

SEQ ID NO: 105 - protein, 8 aa, Artificial Sequence
  Synthetic
GFTFSGFA

SEQ ID NO: 106 - protein, 8 aa, Artificial Sequence
  Synthetic
ISGSGDIT

SEQ ID NO: 107 - protein, 16 aa, Artificial Sequence
  Synthetic
GKDPYNWNHGVYGMDV

SEQ ID NO: 108 - protein, 6 aa, Artificial Sequence
  Synthetic
QSISTY

SEQ ID NO: 109 - protein, 3 aa, Artificial Sequence
  Synthetic
GAS

SEQ ID NO: 110 - protein, 9 aa, Artificial Sequence
  Synthetic
QQSYNIPLT

SEQ ID NO: 111 - protein, 123 aa, Artificial Sequence
  Synthetic
EVQLLESGGGLIQPGGSLRLSCAASGFTFSGFAMSWVRQAPGKGLEWVSGISGSGDITYYGDSVKGRFTISRDNSKSTLYLQMNSLRDADTAVYYCGKDPYNWNHGVYGMDVWGQGTTVTVSS

SEQ ID NO: 112 - protein, 107 aa, Artificial Sequence
  Synthetic
DIQMTQSPSSLSASVGDRVTITCRASQSISTYLNWYQQKPGKAPKFLIYGASSLQSGVPSRFSGSGSGTDFSLTISSLHPGDFATYYCQQSYNIPLTFGGGTKVEIK

SEQ ID NO: 113 - protein, 8 aa, Artificial Sequence
  Synthetic
GFAFHTYD

SEQ ID NO: 114 - protein, 8 aa, Artificial Sequence
  Synthetic
VVGSGINT

SEQ ID NO: 115 - protein, 12 aa, Artificial Sequence
  Synthetic
AKDSSSWFSLHY

SEQ ID NO: 116 - protein, 6 aa, Artificial Sequence
  Synthetic
QSISNW

SEQ ID NO: 117 - protein, 3 aa, Artificial Sequence
  Synthetic
KAS

SEQ ID NO: 118 - protein, 9 aa, Artificial Sequence
  Synthetic
QQYRSYAYT

SEQ ID NO: 119 - protein, 119 aa, Artificial Sequence
  Synthetic
EVQLLESGGGLVQPGGSLRLSCAASGFAFHTYDMTWVRQAPGKGLEWVSGVVGSGINTYYADSVKGRFTISRDNSKSTLYLQMNSLRAEDTAVYYCAKDSSSWFSLHYWGQGTLVTVSS

SEQ ID NO: 120 - protein, 107 aa, Artificial Sequence
  Synthetic
DIQMTQSPSTLSASVGDRVTITCRASQSISNWLAWYQQKPGKAPKLLIYKASSLESGVPSRFSGSGSGTDFTLTISSLQPDDFASYYCQQYRSYAYTFGQGTKLEIT

SEQ ID NO: 121 - protein, 8 aa, Artificial Sequence
  Synthetic
GFTFSDYY

SEQ ID NO: 122 - protein, 8 aa, Artificial Sequence
  Synthetic
IDSSAGAT

SEQ ID NO: 123 - protein, 13 aa, Artificial Sequence
  Synthetic
ARERRSGHKIFDC

SEQ ID NO: 124 - protein, 12 aa, Artificial Sequence
  Synthetic
QNILYSSNNKNY

SEQ ID NO: 125 - protein, 3 aa, Artificial Sequence
  Synthetic
WSS

SEQ ID NO: 126 - protein, 9 aa, Artificial Sequence
  Synthetic
QQYYSSPYN

SEQ ID NO: 127 - protein, 120 aa, Artificial Sequence
  Synthetic
QVQLVESGGDLVKPGGSLRLSCVASGFTFSDYYMNWIRLPPGKGLEWISYIDSSAGATYYADSVKGRFTVSRDDAKNSLFLHMNSLRAEDTAVYYCARERRSGHKIFDCWGPGTLVTVSS

SEQ ID NO: 128 - protein, 113 aa, Artificial Sequence; identical to SEQ ID NO: 16
  Synthetic

SEQ ID NO: 129 - protein, 8 aa, Artificial Sequence
  Synthetic
GFTFSDYY

SEQ ID NO: 130 - protein, 8 aa, Artificial Sequence
  Synthetic
IDSSAGAI

SEQ ID NO: 131 - protein, 13 aa, Artificial Sequence
  Synthetic
ARERRDGHKIFDY

SEQ ID NO: 132 - protein, 12 aa, Artificial Sequence
  Synthetic
QNILYSSNNKNY

SEQ ID NO: 133 - protein, 3 aa, Artificial Sequence
  Synthetic
WSS

SEQ ID NO: 134 - protein, 9 aa, Artificial Sequence
  Synthetic
QQYYSSPYN

SEQ ID NO: 135 - protein, 120 aa, Artificial Sequence
  Synthetic
QVHLVESGGDLVKPGGSLRLSCVASGFTFSDYYMNWIRLPPGKGLEWISYIDSSAGAIYYADSVKGRFTVSRDDAKNSMFLHMNSLRADDTAVYYCARERRDGHKIFDYWGPGTLVTVSS

SEQ ID NO: 136 - protein, 113 aa, Artificial Sequence; identical to SEQ ID NO: 16
  Synthetic

SEQ ID NO: 137 - protein, 8 aa, Artificial Sequence
  Synthetic
GFTFSDYY

SEQ ID NO: 138 - protein, 8 aa, Artificial Sequence
  Synthetic
IDSSAGAI

SEQ ID NO: 139 - protein, 13 aa, Artificial Sequence
  Synthetic
ARERREGHKIFDF

SEQ ID NO: 140 - protein, 12 aa, Artificial Sequence
  Synthetic
QNILYSSNNKNY

SEQ ID NO: 141 - protein, 3 aa, Artificial Sequence
  Synthetic
WSS

SEQ ID NO: 142 - protein, 9 aa, Artificial Sequence
  Synthetic
QQYYSSPYN

SEQ ID NO: 143 - protein, 120 aa, Artificial Sequence
  Synthetic
QVQLVESGGDLVKPGGSLRLSCVASGFTFSDYYMNWIRLPPGKGLEWISYIDSSAGAIYYADSVKGRFTVSRDDAKNSLFLHMNSLRADDTAVYYCARERREGHKIFDFWGPGTLVTVSS

SEQ ID NO: 144 - protein, 113 aa, Artificial Sequence
  Synthetic
DIVMTQSPDSLALSLGERATINCKSSQNILYSSNNKNYLACYQQKAGQPPKLLIFWSSTRESGVPDRFSGSGSGTDFTLTISSLQAEDVAVYYCQQYYSSPYNFGRGTTLEIK

SEQ ID NO: 145 - protein, 8 aa, Artificial Sequence
  Synthetic
GFTFHNYA

SEQ ID NO: 146 - protein, 8 aa, Artificial Sequence
  Synthetic
ISGSGGTA

SEQ ID NO: 147 - protein, 14 aa, Artificial Sequence
  Synthetic
AKDRFLEWVEGFDS

SEQ ID NO: 148 - protein, 6 aa, Artificial Sequence
  Synthetic
QSIGSF

SEQ ID NO: 149 - protein, 3 aa, Artificial Sequence
  Synthetic
AAS

SEQ ID NO: 150 - protein, 9 aa, Artificial Sequence
  Synthetic
QQSYNTPLT

SEQ ID NO: 151 - protein, 121 aa, Artificial Sequence
  Synthetic
EVQLLESGGGLVQPGGSLRLSCAASGFTFHNYAMSWVRQAPGKGLEWVSGISGSGGTAYYADSVKGRFTISRDNSKNTLYLQMNSLRADDTALYYCAKDRFLEWVEGFDSWGQGTLVTVSS

SEQ ID NO: 152 - protein, 107 aa, Artificial Sequence
  Synthetic
DIQMTQSPSSLSASVGDRVTITCRASQSIGSFLIWYQQKPGKAPKLLIYAASSLQSGVPSRFSGSGSGTDFTLIISSLQPEDFATYYCQQSYNTPLTFGGGTKVEIK

SEQ ID NO: 153 - protein, 8 aa, Artificial Sequence
  Synthetic
GFTFSSYE

SEQ ID NO: 154 - protein, 8 aa, Artificial Sequence
  Synthetic
ISSGGSSI

SEQ ID NO: 155 - protein, 15 aa, Artificial Sequence
  Synthetic
ARVGGSYYYSYAMDV

SEQ ID NO: 156 - protein, 6 aa, Artificial Sequence
  Synthetic
QDISSY

SEQ ID NO: 157 - protein, 3 aa, Artificial Sequence
  Synthetic
AAS

SEQ ID NO: 158 - protein, 9 aa, Artificial Sequence
  Synthetic
QQPNNYPFS

SEQ ID NO: 159 - protein, 122 aa, Artificial Sequence
  Synthetic
EVQLVESGGGLVQPGGSLRLSCAASGFTFSSYEMNWVRQAPGKGLEWVSYISSGGSSIKYADSVRGRFTFSRDNAENSVHLQMNSLRGEDTAVYYCARVGGSYYYSYAMDVWGQGTAVTVSS

SEQ ID NO: 160 - protein, 107 aa, Artificial Sequence
  Synthetic
DIQLTQSPSFLSASVGDRVTITCWASQDISSYLAWYQQKPGKAPKLLIYAASTLQSGVPSRFSGSGSGTEFTLTISSLQPEDFATYYCQQPNNYPFSFGPGTKVDVK

SEQ ID NO: 161 - protein, 8 aa, Artificial Sequence
  Synthetic
GFTFSSYE

SEQ ID NO: 162 - protein, 8 aa, Artificial Sequence
  Synthetic
ISSGGSSI

SEQ ID NO: 163 - protein, 15 aa, Artificial Sequence
  Synthetic
ARVGGSYYYSYAMDV

SEQ ID NO: 164 - protein, 6 aa, Artificial Sequence
  Synthetic
QDISSY

SEQ ID NO: 165 - protein, 3 aa, Artificial Sequence
  Synthetic
AAS

SEQ ID NO: 166 - protein, 9 aa, Artificial Sequence
  Synthetic
QQPNNYPFS

SEQ ID NO: 167 - protein, 122 aa, Artificial Sequence
  Synthetic
EVQLVESGGGLVQPGGSLRLSCAASGFTFSSYEMNWVRQAPGKGLEWVSYISSGGSSIKYADSVRGRFTFSRDNAENSVFLQMNSLRGEDTAVYYCARVGGSYYYSYAMDVWGQGTAVTVSS

SEQ ID NO: 168 - protein, 107 aa, Artificial Sequence; identical to SEQ ID NO: 160
  Synthetic

SEQ ID NO: 169 - protein, 8 aa, Artificial Sequence
  Synthetic
GFTFSSFA

SEQ ID NO: 170 - protein, 8 aa, Artificial Sequence
  Synthetic
ISASGGTT

SEQ ID NO: 171 - protein, 14 aa, Artificial Sequence
  Synthetic
AKDRFLEWVEGFDP

SEQ ID NO: 172 - protein, 6 aa, Artificial Sequence
  Synthetic
QSIGRF

SEQ ID NO: 173 - protein, 3 aa, Artificial Sequence
  Synthetic
ATS

SEQ ID NO: 174 - protein, 9 aa, Artificial Sequence
  Synthetic
QQSYITPLT

SEQ ID NO: 175 - protein, 121 aa, Artificial Sequence
  Synthetic
EVQLLESGGGLVQPGGSLRLSCAVSGFTFSSFAMSWVRQAPGKGLEWVSGISASGGTTNYADSVKGRFTISRDNSKNTLYLQMSSLRAEDTAEYYCAKDRFLEWVEGFDPWGQGILVTVSP

SEQ ID NO: 176 - protein, 107 aa, Artificial Sequence
  Synthetic
DIQMTQSPSSLSASVGDRVTITCRASQSIGRFLIWYQQKPGKAPKLLLYATSSLQSGVPARFTGSGSGTDFTLTIGSLQPEDFATYYCQQSYITPLTFGGGTKVEIK

SEQ ID NO: 177 - protein, 8 aa, Artificial Sequence
  Synthetic
GFTFSSFA

SEQ ID NO: 178 - protein, 8 aa, Artificial Sequence
  Synthetic
ISASGGTT

SEQ ID NO: 179 - protein, 14 aa, Artificial Sequence
  Synthetic
AKDRFLEWVEGFDP

SEQ ID NO: 180 - protein, 6 aa, Artificial Sequence
  Synthetic
QSIGRF

SEQ ID NO: 181 - protein, 3 aa, Artificial Sequence
  Synthetic
ATS

SEQ ID NO: 182 - protein, 9 aa, Artificial Sequence
  Synthetic
QQSYITPLT

SEQ ID NO: 183 - protein, 121 aa, Artificial Sequence
  Synthetic
EVQLLESGGGLVQPGGSLRLSCAASGFTFSSFAMSWVRQAPGKGLEWVSGISASGGTTNYADSVKGRFTISRDNSKNTLYLQMSSLRAEDTAEYYCAKDRFLEWVEGFDPWGQGILVTVSP

SEQ ID NO: 184 - protein, 107 aa, Artificial Sequence; identical to SEQ ID NO: 176
  Synthetic

SEQ ID NO: 185 - protein, 25 aa, Artificial Sequence
  Synthetic
QVQLVESGGDLVKPGGSLRLSCVAS

SEQ ID NO: 186 - protein, 17 aa, Artificial Sequence
  Synthetic
MNWIRLPPGKGLEWVSY

SEQ ID NO: 187 - protein, 38 aa, Artificial Sequence
  Synthetic
YYADSVRGRFTVSRDDAKNSLFLHMNSLRADDTAVYYC

SEQ ID NO: 188 - protein, 11 aa, Artificial Sequence
  Synthetic
WGPGTLVTVSS

SEQ ID NO: 189 - protein, 26 aa, Artificial Sequence
  Synthetic
DIVMTQSPDSLAVSLGERATINCKSS

SEQ ID NO: 190 - protein, 17 aa, Artificial Sequence
  Synthetic
LAWYQQKAGQPPKLLIF

SEQ ID NO: 191 - protein, 36 aa, Artificial Sequence
  Synthetic
TRESGVPDRFSGSGSGTDFTLTINSLQAEDVAVYYC

SEQ ID NO: 192 - protein, 10 aa, Artificial Sequence
  Synthetic
FGRGTTLEIK

SEQ ID NO: 193 - protein, 25 aa, Artificial Sequence
  Synthetic
QVHLVESGGDLVKPGGSLRLSCVAS

SEQ ID NO: 194 - protein, 17 aa, Artificial Sequence
  Synthetic
MNWIRMPPGKGLEWISY

SEQ ID NO: 195 - protein, 38 aa, Artificial Sequence
  Synthetic
YYADSVKGRFTVSRDDAKNSLFLHMNNLRADDTAVYYC

SEQ ID NO: 196 - protein, 36 aa, Artificial Sequence
  Synthetic
TRESGVPDRFSGSGSGTDFTLTISSLQAEDVAVYYC

SEQ ID NO: 197 - protein, 25 aa, Artificial Sequence
  Synthetic
EVQLVQSGAEVKKPGESLKISCKGS

SEQ ID NO: 198 - protein, 17 aa, Artificial Sequence
  Synthetic
IGWVRQMPGKGLEWMGI

SEQ ID NO: 199 - protein, 38 aa, Artificial Sequence
  Synthetic
RYSPSFQGQVTISADKSISTAYLQWSSLKASDTAMYYC

SEQ ID NO: 200 - protein, 11 aa, Artificial Sequence
  Synthetic
WGQGTLVTVSS

SEQ ID NO: 201 - protein, 26 aa, Artificial Sequence
  Synthetic
DIQMTQSPSSLSASVGDRLTITCRAS

SEQ ID NO: 202 - protein, 17 aa, Artificial Sequence
  Synthetic
LNWYQQNPGKAPKVLIY

SEQ ID NO: 203 - protein, 36 aa, Artificial Sequence
  Synthetic
SLESGVPSRFSGSGSGTDFTLTISSLQPEDFATYYC

SEQ ID NO: 204 - protein, 10 aa, Artificial Sequence
  Synthetic
FGQGTKVEIK

SEQ ID NO: 205 - protein, 25 aa, Artificial Sequence
  Synthetic
EVQLLESGGGLAQPGKSLRLSCAAS

SEQ ID NO: 206 - protein, 17 aa, Artificial Sequence
  Synthetic
MSWVRQAPGKGLEWVSG

SEQ ID NO: 207 - protein, 38 aa, Artificial Sequence
  Synthetic
YYADSVKGRFTISRDNSKSTLYLQMNSLGAEDTAVYYC

SEQ ID NO: 208 - protein, 11 aa, Artificial Sequence
  Synthetic
WGQGTTVTVSS

SEQ ID NO: 209 - protein, 26 aa, Artificial Sequence [sequence omitted]

SEQ ID NO: 210 - protein, 17 aa, Artificial Sequence
  Synthetic
LNWYQQKPGKAPKFLIY

SEQ ID NO: 211 - protein, 36 aa, Artificial Sequence
  Synthetic
  misc_feature (17)..(17): Xaa can be any naturally occurring amino acid
SLQSGVPSRFSGSGCGXDFTLTISSLQREDFAAYYC

SEQ ID NO: 212 - protein, 10 aa, Artificial Sequence
  Synthetic
FGGGTKVEIK

SEQ ID NO: 213 - protein, 25 aa, Artificial Sequence
  Synthetic
QVQLVESGGGLVKPGGSLRLSCVAS

SEQ ID NO: 214 - protein, 17 aa, Artificial Sequence
  Synthetic
MNWIRLPPGRGLQWISY

SEQ ID NO: 215 - protein, 38 aa, Artificial Sequence
  Synthetic
YYADSVKGRFTVSRDDAKNSLYLQMNSLRADDTAVYYC

SEQ ID NO: 216 - protein, 17 aa, Artificial Sequence
  Synthetic
LGWYQQKAGQPPKLLIY

SEQ ID NO: 217 - protein, 36 aa, Artificial Sequence
  Synthetic
TRNSGVPDRFSGSGSGTDFTLTISSLQAEDVAVYYC

SEQ ID NO: 218 - protein, 10 aa, Artificial Sequence
  Synthetic
FGQGTNLEIK

SEQ ID NO: 219 - protein, 38 aa, Artificial Sequence
  Synthetic
RYSPSFQGQVTLSADKSISTAYLQWNSLKASDTAMYYC

SEQ ID NO: 220 - protein, 11 aa, Artificial Sequence
  Synthetic
WGQGTLVTVAS

SEQ ID NO: 221 - protein, 17 aa, Artificial Sequence
  Synthetic
LNWYQQKPGTAPKLLIY

SEQ ID NO: 222 - protein, 36 aa, Artificial Sequence
  Synthetic
SLQGGVPSRFSGSGSGTDFTLTISSLQPEDFATYYC

SEQ ID NO: 223 - protein, 17 aa, Artificial Sequence
  Synthetic
MNWIRLPPGKGLEWISY

SEQ ID NO: 224 - protein, 38 aa, Artificial Sequence
  Synthetic
YYADSVKGRFTVSRDDAKNSLFLHMNSLRADDTAVYYC

SEQ ID NO: 225 - protein, 17 aa, Artificial Sequence
  Synthetic
LAWYQQKTGQPPKLLIF

SEQ ID NO: 226 - protein, 25 aa, Artificial Sequence
  Synthetic
EVQLVESGGDLVQPGRSLRLSCAAS

SEQ ID NO: 227 - protein, 17 aa, Artificial Sequence
  Synthetic
MHWVRQGPGKGLEWVSG

SEQ ID NO: 228 - protein, 38 aa, Artificial Sequence
  Synthetic
GYADSVKGRFTISRDNAKNSLYLRMNSLRADDTAFYYC

SEQ ID NO: 229 - protein, 11 aa, Artificial Sequence
  Synthetic
WGHGTMVTVSS

SEQ ID NO: 230 - protein, 17 aa, Artificial Sequence
  Synthetic
LDWYQQKPGQPPKLLIY

SEQ ID NO: 231 - protein, 36 aa, Artificial Sequence
  Synthetic
SRESGVPDRFSGSGSGTDFTLTISSLQAEDVAVYYC

SEQ ID NO: 232 - protein, 17 aa, Artificial Sequence
  Synthetic
MNWIRLPPGRGLEWISY

SEQ ID NO: 233 - protein, 38 aa, Artificial Sequence
  Synthetic
YYADSVKGRFTVSRDDAKKSLFLYMNSLRADDTAVYYC

SEQ ID NO: 234 - protein, 25 aa, Artificial Sequence
  Synthetic
EVQLLESGGGLVQPGGSLRLSCAAS

SEQ ID NO: 235 - protein, 38 aa, Artificial Sequence
  Synthetic
YYADSVKGRFTISRDNSKNTLYLQMNSLRAEDTAIYYC

SEQ ID NO: 236 - protein, 11 aa, Artificial Sequence
  Synthetic
WGQGTMVTVSS

SEQ ID NO: 237 - protein, 26 aa, Artificial Sequence
  Synthetic
DIQMTQSPSSLSASVGGRVTITCRTS

SEQ ID NO: 238 - protein, 17 aa, Artificial Sequence
  Synthetic
LNWYQQKAGKAPKLLIY

SEQ ID NO: 239 - protein, 36 aa, Artificial Sequence
  Synthetic
NLQSGVPSRFTGSGSGTDFTLTISSLQPEDFAAYYC

SEQ ID NO: 240 - protein, 10 aa, Artificial Sequence
  Synthetic
FGPGTKVAFK

SEQ ID NO: 241 - protein, 25 aa, Artificial Sequence [sequence omitted]

SEQ ID NO: 242 - protein, 17 aa, Artificial Sequence
  Synthetic
MNWVRQAPGKGLEWVSY

SEQ ID NO: 243 - protein, 38 aa, Artificial Sequence
  Synthetic
KYADSVRGRFAFSRDNAENSVHLQMNSLRAEDTAVYYC

SEQ ID NO: 244 - protein, 11 aa, Artificial Sequence
  Synthetic
WGQGTAVTVSS

SEQ ID NO: 245 - protein, 26 aa, Artificial Sequence
  Synthetic
DIQLTQSPSFLSASVGDRVTITCWAS

SEQ ID NO: 246 - protein, 17 aa, Artificial Sequence
  Synthetic
LAWYQQKPGKAPKLLIY

SEQ ID NO: 247 - protein, 36 aa, Artificial Sequence
  Synthetic
TLQSGVPSRFTGSGSGTEFTLTISSLQPEDFATYYC

SEQ ID NO: 248 - protein, 10 aa, Artificial Sequence
  Synthetic
FGPGTKVDVK

SEQ ID NO: 249 - protein, 36 aa, Artificial Sequence
  Synthetic
IRESGVPDRFSGSGSGTDFTLTISSLQAEDVAVYYC

SEQ ID NO: 250 - protein, 25 aa, Artificial Sequence
  Synthetic
EVQLLESGGGLIQPGGSLRLSCAAS

SEQ ID NO: 251 - protein, 38 aa, Artificial Sequence
  Synthetic
YYGDSVKGRFTISRDNSKSTLYLQMNSLRDADTAVYYC

SEQ ID NO: 252 - protein, 36 aa, Artificial Sequence
  Synthetic
  misc_feature (26)..(26): Xaa can be any naturally occurring amino acid
SLQSGVPSRFSGSGCGTDFSLTISSXHPGDFATYYC

SEQ ID NO: 253 - protein, 17 aa, Artificial Sequence
  Synthetic
MTWVRQAPGKGLEWVSG

SEQ ID NO: 254 - protein, 38 aa, Artificial Sequence
  Synthetic
YYADSVKGRFTISRDNSKSTLYLQMNSLRAEDTAVYYC

SEQ ID NO: 255 - protein, 26 aa, Artificial Sequence
  Synthetic
DIQMTQSPSTLSASVGDRVTITCRAS

SEQ ID NO: 256 - protein, 36 aa, Artificial Sequence
  Synthetic
SLESGVPSRFSGSGSGTDFTLTISSLQPDDFASYYC

SEQ ID NO: 257 - protein, 10 aa, Artificial Sequence
  Synthetic
FGQGTKLEIT

SEQ ID NO: 258 - protein, 38 aa, Artificial Sequence
  Synthetic
YYADSVKGRFTVSRDDAKNSLFLHMNSLRAEDTAVYYC

SEQ ID NO: 259 - protein, 38 aa, Artificial Sequence
  Synthetic
YYADSVKGRFTVSRDDAKNSMFLHMNSLRADDTAVYYC

SEQ ID NO: 260 - protein, 26 aa, Artificial Sequence
  Synthetic
DIVMTQSPDSLALSLGERATINCKSS

SEQ ID NO: 261 - protein, 17 aa, Artificial Sequence
  Synthetic
LACYQQKAGQPPKLLIF

SEQ ID NO: 262 - protein, 38 aa, Artificial Sequence
  Synthetic
YYADSVKGRFTISRDNSKNTLYLQMNSLRADDTALYYC

SEQ ID NO: 263 - protein, 17 aa, Artificial Sequence
  Synthetic
LIWYQQKPGKAPKLLIY

SEQ ID NO: 264 - protein, 36 aa, Artificial Sequence
  Synthetic
SLQSGVPSRFSGSGSGTDFTLIISSLQPEDFATYYC

SEQ ID NO: 265 - protein, 38 aa, Artificial Sequence
  Synthetic
KYADSVRGRFTFSRDNAENSVHLQMNSLRGEDTAVYYC

SEQ ID NO: 266 - protein, 36 aa, Artificial Sequence
  Synthetic
  misc_feature (15)..(15): Xaa can be any naturally occurring amino acid
TLQSGVPSRFSGSGXGTEFTLTISSLQREDFATYYC

SEQ ID NO: 267 - protein, 38 aa, Artificial Sequence
  Synthetic
KYADSVRGRFTFSRDNAENSVFLQMNSLRGEDTAVYYC

SEQ ID NO: 268 - protein, 36 aa, Artificial Sequence
  Synthetic
TLQSGVPSRFSGSGSGTEFTLTISSLQPEDFATYYC

SEQ ID NO: 269 - protein, 25 aa, Artificial Sequence
  Synthetic
EVQLLESGGGLVQPGGSLRLSCAVS

SEQ ID NO: 270 - protein, 38 aa, Artificial Sequence
  Synthetic
NYADSVKGRFTISRDNSKNTLYLQMSSLRAEDTAEYYC

SEQ ID NO: 271 - protein, 11 aa, Artificial Sequence
  Synthetic
WGQGILVTVSP

SEQ ID NO: 272 - protein, 17 aa, Artificial Sequence
  Synthetic
LIWYQQKPGKAPKLLLY

SEQ ID NO: 273 - protein, 36 aa, Artificial Sequence
  Synthetic
SLQSGVPARFTGSGSGTDFTLTIGSLQPEDFATYYC

SEQ ID NO: 274 - protein, 330 aa, Artificial Sequence [sequence omitted]

SEQ ID NO: 275 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 276 - protein, 377 aa, Artificial Sequence
  Synthetic
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 277 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 278 - protein, 353 aa, Artificial Sequence
  Synthetic
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 279 - protein, 340 aa, Artificial Sequence
  Synthetic
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 280 - protein, 384 aa, Artificial Sequence
  Synthetic
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 281 - protein, 453 aa, Artificial Sequence
  Synthetic
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 282 - protein, 428 aa, Artificial Sequence
  Synthetic
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 283 - protein, 324 aa, Artificial Sequence
  Synthetic
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 284 - protein, 330 aa, Artificial Sequence
  Synthetic
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 285 - protein, 335 aa, Artificial Sequence
  Synthetic
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 286 - protein, 335 aa, Artificial Sequence
  Synthetic
AKTTAPSVYPLVPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPHQRVPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 287 - protein, 344 aa, Artificial Sequence
  Synthetic
ESARNPTIYPLTLPPALSSDPVIIGCLIHDYFPSGTMNVTWGKSGKDITTVNFPPALASGGRYTMSNQLTLPAVECPEGESVKCSVQHDSNPVQELDVNCSGPTPPPPITIPSCQPSLSLQRPALEDLLLGSDASITCTLNGLRNPEGAVFTWEPSTGKDAVQKKAVQNSCGCYSVSSVLPGCAERWNSGASFKCTVTHPESGTLTGTIAKVTVNTFPPQVHLLPPPSEELALNELLSLTCLVRAFNPKEVLVRWLHGNEELSPESYLVFEPLKEPGEGATTYLVTSVLRVSAETWKQGDQYSCMVGHEALPMNFTQKTIDRLSGKPTNVSVSVIMSEGDGICY

SEQ ID NO: 288 - protein, 454 aa, Artificial Sequence
  Synthetic
SQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 289 - protein, 257 aa, Artificial Sequence
  Synthetic
DKKEPDMFLLSECKAPEENEKINLGCLVIGSQPLKISWEPKKSSIVEHVFPSEMRNGNYTMVLQVTVLASELNLNHTCTINKPKRKEKPFKFPESWDSQSSKRVTPTLQAKNHSTEATKAITTKKDIEGAMAPSNLTVNILTTSTHPEMSSWLLCEVSGFFPENIHLMWLGVHSKMKSTNFVTANPTAQPGGTFQTWSVLRLPVALSSSLDTYTCVVEHEASKTKLNASKSLAISGCYHLLPESDGPSRRPDGPALA

SEQ ID NO: 290 - protein, 421 aa, Artificial Sequence
  Synthetic
SIRNPQLYPLKPCKGTASMTLGCLVKDYFPNPVTVTWYSDSLNMSTVNFPALGSELKVTTSQVTSWGKSAKNFTCHVTHPPSFNESRTILVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 291 - protein, 107 aa, Artificial Sequence [sequence omitted]

SEQ ID NO: 292 - protein, 106 aa, Artificial Sequence
  Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 293 - protein, 106 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 294 - protein, 106 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 295 - protein, 106 aa, Artificial Sequence
  Synthetic
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 296 - protein, 106 aa, Artificial Sequence
  Synthetic
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 297 - protein, 107 aa, Artificial Sequence
  Synthetic
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 298 - protein, 106 aa, Artificial Sequence
  Synthetic
  misc_feature (1)..(1): Xaa can be any naturally occurring amino acid
XQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 299 - protein, 105 aa, Artificial Sequence
  Synthetic
GQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 300 - protein, 104 aa, Artificial Sequence
  Synthetic
QPKSTPTLTMFPPSPEELQENKATLVCLISNFSPSGVTVAWKANGTPITQGVDTSNPTKEDNKYMASSFLHLTSDQWRSHNSFTCQVTHEGDTVEKSLSPAECL

SEQ ID NO: 301 - protein, 104 aa, Artificial Sequence
  Synthetic
  misc_feature (1)..(1): Xaa can be any naturally occurring amino acid
XQPKATPSVNLFPPSSEELKTKKATLVCMITEFYAAAVRVAWKADGTPFTQGVETTQPPKQRDNMASSYLLFTAEAWESHSSYSCHVTHEGNTVEKSLSRAECS

SEQ ID NO: 302 - DNA, 24 bp, Artificial Sequence
  Synthetic
ggattcaccttcagtgactactac

SEQ ID NO: 303 - DNA, 24 bp, Artificial Sequence
  Synthetic
attgatagtagtgctggtgccatt

SEQ ID NO: 304 - DNA, 39 bp, Artificial Sequence
  Synthetic
gcgagagagcgtagagatggccacaaaatctttgactgt

SEQ ID NO: 305 - DNA, 36 bp, Artificial Sequence
  Synthetic
cagaatattttgtacagttccaacaataagaactac

SEQ ID NO: 306 - DNA, 9 bp, Artificial Sequence
  Synthetic
tggtcatct

SEQ ID NO: 307 - DNA, 27 bp, Artificial Sequence
  Synthetic
cagcaatattacagtagtccgtataat

SEQ ID NO: 308 - DNA, 360 bp, Artificial Sequence
  Synthetic
caggtgcagctggtggagtctgggggagacttggtcaagcctggagggtccctgagactctcctgtgtagcctcgggattcaccttcagtgactactacatgaactggatccgcctgcctccagggaagggcctggagtgggtttcatacattgatagtagtgctggtgccatttactatgcagactctgtgaggggccgattcaccgtctccagggacgacgccaagaattcactatttctgcacatgaacagcctgagagccgatgacacggccgtttattactgtgcgagagagcgtagagatggccacaaaatctttgactgttggggcccgggaaccctggtcaccgtctcttca

SEQ ID NO: 309 - DNA, 339 bp, Artificial Sequence
  Synthetic
gacatcgtgatgacccagtctccagactccctggctgtgtctctgggcgagagggccaccatcaactgcaagtccagccagaatattttgtacagttccaacaataagaactacttagcttggtaccagcagaaagcaggacagcctcctaagttgctcattttctggtcatctacccgggaatccggggtccctgaccgattcagtggcagcgggtctgggacagatttcactctcaccatcaacagcctgcaggctgaagatgtggcagtttattactgtcagcaatattacagtagtccgtataattttggccgggggaccacactggagatcaaa

SEQ ID NO: 310 - DNA, 36 bp, Artificial Sequence
  Synthetic
cagaatattttatacagttccaacaataagaactat

SEQ ID NO: 311 - DNA, 360 bp, Artificial Sequence
  Synthetic
caggtgcacctggtggagtctgggggagacttggtcaagcctggagggtccctgagactctcctgtgtagcctctggattcaccttcagtgactactacatgaactggatccgtatgcctccagggaagggcctggagtggatttcatacattgatagtagtgctggtgccatttactatgcagactctgtgaagggccgattcaccgtctccagggacgacgccaagaactcactgtttctacacatgaacaacctgagagccgatgacacggccgtttattactgtgcgagagagcgtagagatggccacaaaatctttgactgttggggcccgggaaccctggtcaccgtctcctca

SEQ ID NO: 312 - DNA, 339 bp, Artificial Sequence
  Synthetic
gacatcgtgatgacccagtctccagactccctggctgtgtctctgggcgagagggccaccatcaactgcaagtccagccagaatattttatacagttccaacaataagaactatttagcttggtaccagcagaaagcaggacagcctcctaagttgctcattttctggtcatctacccgggaatccggggtccctgaccgattcagtggcagcgggtctgggacagatttcactctcaccatcagcagcctgcaggctgaagatgtggcagtttattactgtcagcaatattacagtagtccgtataattttggccgggggaccacactggagatcaaa

SEQ ID NO: 313 - DNA, 24 bp, Artificial Sequence
  Synthetic
ggatacagttttacatactactgg

SEQ ID NO: 314 - DNA, 24 bp, Artificial Sequence
  Synthetic
atctatcctggtgactctgatacc

SEQ ID NO: 315 - DNA, 36 bp, Artificial Sequence
  Synthetic
actacacatgcgcgcaactggaacaacgtggcctac

SEQ ID NO: 316 - DNA, 18 bp, Artificial Sequence
  Synthetic
cagaacattaacaccttt

SEQ ID NO: 317 - DNA, 9 bp, Artificial Sequence
  Synthetic
gctgcatcc

SEQ ID NO: 318 - DNA, 27 bp, Artificial Sequence
  Synthetic
caacagagttctagtaccccgtggacg

SEQ ID NO: 319 - DNA, 357 bp, Artificial Sequence
  Synthetic
gaggtgcagctggtgcagtctggagcagaggtgaaaaagcccggggagtctctgaagatctcctgtaagggttctggatacagttttacatactactggatcggctgggtgcgccagatgcccgggaaaggcctggagtggatggggatcatctatcctggtgactctgataccagatacagcccgtccttccaaggccaggtcaccatctcagccgacaagtccatcagtaccgcctatttgcagtggagcagcctgaaggcctcggacaccgccatgtattactgtactacacatgcgcgcaactggaacaacgtggcctactggggccagggaaccctggtcaccgtctcctca

SEQ ID NO: 320 - DNA, 322 bp, Artificial Sequence
  Synthetic
gacatccagatgacccagtctccatcctccctgtctgcatctgtcggagacagactcaccatcacttgccgggcaagtcagaacattaacacctttttaaattggtatcaacagaacccagggaaagcccctaaggtcctgatctatgctgcatccagtttggaaagtggggtcccatcaaggttcagtggcagtggatctgggacagatttcactctcaccatcagcagtctgcaacctgaagattttgcaacttactactgtcaacagagttctagtaccccgtggacgttcggccaagggaccaaggtggaaatcaaac

SEQ ID NO: 321 - DNA, 24 bp, Artificial Sequence
  Synthetic
ggattcacctttagtggcttcgcc

SEQ ID NO: 322 - DNA, 24 bp, Artificial Sequence
  Synthetic
attagtgggagtggtgatatcaca

SEQ ID NO: 323 - DNA, 48 bp, Artificial Sequence
  Synthetic
gcgaaagacccatataactggaaccacggggtctacggcatggacgtc

SEQ ID NO: 324 - DNA, 18 bp, Artificial Sequence
  Synthetic
cagagcattagcagctat

SEQ ID NO: 325 - DNA, 9 bp, Artificial Sequence
  Synthetic
ggtgcatcc

SEQ ID NO: 326 - DNA, 27 bp, Artificial Sequence
  Synthetic
caacagagtttcaatgcccctctcact

SEQ ID NO: 327 - DNA, 369 bp, Artificial Sequence
  Synthetic
gaggtgcagcttttggagtcggggggaggcttggcacagccggggaagtccctgagactctcctgtgcagcctctggattcacctttagtggcttcgccatgagctgggtccgccaggctcctgggaaggggctggagtgggtctcaggtattagtgggagtggtgatatcacatactatgcagactccgtgaagggccggttcaccatttccagagacaattccaagagtacgctgtatcttcaaatgaacagcctgggagccgaggacacggccgtgtattactgtgcgaaagacccatataactggaaccacggggtctacggcatggacgtctggggccaagggaccacggtcaccgtctcctca

SEQ ID NO: 328 - DNA, 321 bp, Artificial Sequence
  Synthetic
gacatccagatgacccagtctccatcctccctgtctgcatctgtaggagacagagtcaccatcacttgccgggcaagtcagagcattagcagctatttaaattggtatcagcagaaaccagggaaagcccctaaattcctgatctatggtgcatccagtttgcaaagtggggtcccatcaaggttcagtggcagtggatctgggactgatttcactctcaccatcagcagtctgcaacctgaagatttcgcagcttactactgtcaacagagtttcaatgcccctctcactttcggcggcgggaccaaggtggagatcaaa

SEQ ID NO: 329 - DNA, 39 bp, Artificial Sequence
  Synthetic
gcgagagaacgtagagatggccacaaaatctttgactgg

SEQ ID NO: 330 - DNA, 36 bp, Artificial Sequence
  Synthetic
cagaatattttatacacttccaacaataagaactac

SEQ ID NO: 331 - DNA, 27 bp, Artificial Sequence
  Synthetic
catcagtattacagtagtccgtacact

SEQ ID NO: 332 - DNA, 360 bp, Artificial Sequence
  Synthetic
caggtgcagctggtggagtctgggggaggcttggtcaagcctggagggtccctgagactctcctgcgtagcctctggattcaccttcagtgactactacatgaactggatccgcctgcctccagggagggggctgcagtggatttcatacattgatagtagtgctggtgccatttactacgcagactctgtgaagggccgattcaccgtctccagggacgacgccaagaattcactttatctgcaaatgaacagcctgagagccgatgacacggccgtttattactgtgcgagagaacgtagagatggccacaaaatctttgactggtggggccagggaaccctggtcaccgtctcctca

SEQ ID NO: 333 - DNA, 339 bp, Artificial Sequence
  Synthetic
gacatcgtgatgacccagtctccagactccctggctgtgtctctgggcgagagggccaccatcaactgcaagtccagccagaatattttatacacttccaacaataagaactacttaggttggtaccagcagaaagcaggacagcctcctaaactgctcatttactggtcatctacccggaattccggggtccctgaccgcttcagtggcagcgggtctgggacagatttcactctcaccatcagcagcctgcaggctgaggatgtggcagtttattactgtcatcagtattacagtagtccgtacacttttggccaggggaccaacctggagatcaaa

SEQ ID NO: 334 - DNA, 24 bp, Artificial Sequence
  Synthetic
ggatacagttttaccaactactgg

SEQ ID NO: 335 - DNA, 36 bp, Artificial Sequence
  Synthetic
gcgacacatgcgcgcagctggaactacgtggcctac

SEQ ID NO: 336 - DNA, 18 bp, Artificial Sequence
  Synthetic
cagagcgtcaacaacttt

SEQ ID NO: 337 - DNA, 27 bp, Artificial Sequence
  Synthetic
caacagagtaacactaccccgtggacg

SEQ ID NO: 338 - DNA, 358 bp, Artificial Sequence
  Synthetic
gaggtgcagctggtgcagtctggagcagaggtgaaaaagcccggggagtctctgaagatctcctgtaagggttctggatacagttttaccaactactggatcggctgggtgcgccagatgcccgggaaaggcctggagtggatggggatcatctatcctggtgactctgataccagatacagcccgtccttccaaggccaggtcaccctctcagccgacaagtccatcagtaccgcctacctacagtggaacagcctgaaggcctcggacaccgccatgtattactgtgcgacacatgcgcgcagctggaactacgtggcctactggggccagggaaccctggtcaccgtcgcctcat

SEQ ID NO: 339 - DNA, 321 bp, Artificial Sequence
  Synthetic
gacatccagatgacccagtctccatcctccctgtctgcatctgtaggagacagagtcaccatcacttgccgggcaagtcagagcgtcaacaactttttaaattggtatcagcagaaaccagggacagcccctaaactcctgatctatgctgcatccagtttgcaaggtggggtcccatcaaggttcagtggcagtggctctgggacagatttcactctcaccatcagcagtctgcaacctgaagattttgcaacttactactgtcaacagagtaacactaccccgtggacgttcggccaagggaccaaggtggaaatcaaa

SEQ ID NO: 340 - DNA, 36 bp, Artificial Sequence
  Synthetic
cagaatattttatacagttccaacaataagaactac

SEQ ID NO: 341 - DNA, 360 bp, Artificial Sequence
  Synthetic
caggtgcagctggtggagtctgggggagacttggtcaagcctggagggtccctgagactctcctgtgtagcctctggattcaccttcagtgactactacatgaactggatccgcctgcctccagggaagggcctggagtggatttcctacattgatagtagtgctggtgccatttactatgcagactctgtgaagggccgattcaccgtctccagggacgacgccaagaactcactatttctgcacatgaacagcctgagagccgatgacacggccgtttattactgtgcgagagagcgtagagatggccacaaaatctttgactgttggggcccgggaaccctggtcaccgtctcctca

SEQ ID NO: 342 - DNA, 339 bp, Artificial Sequence
  Synthetic
gacatcgtgatgacccagtctccagactccctggctgtgtctctgggcgagagggccaccatcaactgcaagtccagccagaatattttatacagttccaacaataagaactacttagcttggtaccagcagaaaacaggacagcctcctaagttgctcattttctggtcatctacccgggaatccggggtccctgaccgattcagtggcagcgggtctgggacagatttcactctcaccatcagcagcctgcaggctgaggatgtggcagtttattactgtcagcaatattacagtagtccgtataattttggccgggggaccacactggagatcaaa

SEQ ID NO: 343 - DNA, 24 bp, Artificial Sequence
  Synthetic
ggattcacctttgatgattatgcc

SEQ ID NO: 344 - DNA, 24 bp, Artificial Sequence
  Synthetic
attagttggagtggtgttaccatg

SEQ ID NO: 345 - DNA, 33 bp, Artificial Sequence
  Synthetic
gcaagaggggatggaaccaatgcttttgatatc

SEQ ID NO: 346 - DNA, 36 bp, Artificial Sequence
  Synthetic
cagagtgttttgtacaagtccaacaataagaactac

SEQ ID NO: 347 - DNA, 9 bp, Artificial Sequence
  Synthetic
tgggcatct

SEQ ID NO: 348 - DNA, 27 bp, Artificial Sequence
  Synthetic
cagcaatattatagtgttccgctcact

SEQ ID NO: 349 - DNA, 354 bp, Artificial Sequence
  Synthetic
gaagtgcagttggtggagtctgggggagacttggtacagcctggcaggtccctgagactctcctgtgcagcctctggattcacctttgatgattatgccatgcactgggtccggcaaggtccagggaagggcctggagtgggtctcaggcattagttggagtggtgttaccatgggctatgcggactctgtgaagggccgattcaccatttccagagacaacgccaagaactccctgtacctgcgaatgaacagtctgagagctgacgacacggccttctattactgtgcaagaggggatggaaccaatgcttttgatatctggggccacgggacaatggtcaccgtctcttca

SEQ ID NO: 350 - DNA, 339 bp, Artificial Sequence
  Synthetic
gacatcgtgatgacccagtctccagactccctggctgtgtctctgggcgagagggccaccatcaactgcaagtccagccagagtgttttgtacaagtccaacaataagaactacttggattggtatcagcagaaaccaggacagcctcctaagctgctcatttattgggcatcttcccgggaatccggggtccctgaccgattcagtggcagcgggtctgggacagatttcactctgaccatcagcagcctgcaggctgaagatgtggcagtttattactgtcagcaatattatagtgttccgctcactttcggcggagggaccaaggtggagatcaaa

SEQ ID NO: 351 - DNA, 360 bp, Artificial Sequence
  Synthetic
caggtgcagctggtggagtctgggggagacttggtcaagcctggagggtccctgagactctcctgtgtagcctctggattcaccttcagtgactactacatgaactggatccgcctgcctccagggaagggcctggagtggatttcatacattgatagtagtgctggtgccatttactatgcagactctgtgaagggccgattcaccgtctccagggacgacgccaagaactcactatttctacacatgaacaacctgagagccgatgacacggccgtttattactgtgcgagagagcgtagagatggccacaaaatctttgactgttggggcccgggaaccctggtcaccgtctcctca

SEQ ID NO: 352 - DNA, 339 bp, Artificial Sequence
  Synthetic
gacatcgtgatgacccagtctccagactccctggctgtgtctctgggcgagagggccaccatcaactgcaagtccagccagaatattttatacagttccaacaataagaactacttagcttggtaccagcagaaagcaggacagcctcctaagttgctcattttctggtcatctacccgggaatccggggtccctgaccgattcagtggcagcgggtctgggacagatttcactctcaccatcagcagcctgcaggctgaagatgtggcagtttattactgtcagcaatattacagtagtccgtataattttggccgggggaccacactggagatcaaa

SEQ ID NO: 353 - DNA, 39 bp, Artificial Sequence
  Synthetic
gcgagagagcgtagagatggccacaaaatctttgactat

SEQ ID NO: 354 - DNA, 27 bp, Artificial Sequence
  Synthetic
caccaatattacagtagtccgtataat

SEQ ID NO: 355 - DNA, 360 bp, Artificial Sequence
  Synthetic
caggtgcagctggtggagtctgggggagacttggtcaagcctggagggtccctgagactctcctgtgtagcctctggattcaccttcagtgactactacatgaactggatccgcctgcctccagggaggggcctggagtggatttcatacattgatagtagtgctggtgccatttactatgcagactctgtgaagggccgattcaccgtctccagggacgacgccaagaagtcactatttctgtacatgaacagcctgagagccgatgacacggccgtttattactgtgcgagagagcgtagagatggccacaaaatctttgactattggggcccgggaaccctggtcaccgtctcctca

SEQ ID NO: 356 - DNA, 339 bp, Artificial Sequence
  Synthetic
gacatcgtgatgacccagtctccagactccctggctgtgtctctgggcgagagggccaccatcaactgcaagtccagccagaatattttatacagttccaacaataagaactacttagcttggtaccagcagaaagcaggacagcctcctaagttgctcattttctggtcatctacccgggaatccggggtccctgaccgattcagtggcagcgggtctgggacagatttcactctcaccatcagcagcctgcaggctgaagatgtggcagtttattactgtcaccaatattacagtagtccgtataattttggccgggggaccacactggagatcaaa

SEQ ID NO: 357 - DNA, 24 bp, Artificial Sequence
  Synthetic
ggattcacctttagcagctttgcc

SEQ ID NO: 358 - DNA, 24 bp, Artificial Sequence
  Synthetic
attagtggtagtggtggtagcaca

SEQ ID NO: 359 - DNA, 48 bp, Artificial Sequence
  Synthetic
gcgaaagataaaaggaactggaactacgggattgattcttttgatttc

SEQ ID NO: 360 - DNA, 18 bp, Artificial Sequence
  Synthetic
cagagcattagcggctat

SEQ ID NO: 361 - DNA, 9 bp, Artificial Sequence
  Synthetic
gctacatcc

SEQ ID NO: 362 - DNA, 27 bp, Artificial Sequence
  Synthetic
catcagagtcacagtcccccattcact

SEQ ID NO: 363 - DNA, 369 bp, Artificial Sequence
  Synthetic
gaggtgcagttgttggagtctgggggaggcttggtacagcctggggggtccctgagactctcctgtgcagcctctggattcacctttagcagctttgccatgagctgggtccgccaggctccagggaaggggctggagtgggtctcaggtattagtggtagtggtggtagcacatactacgcagactccgtgaagggccggttcaccatctccagagacaattccaagaacacgctgtatctgcaaatgaacagcctgagagccgaggacacggccatatattactgtgcgaaagataaaaggaactggaactacgggattgattcttttgatttctggggccaagggacgatggtcaccgtctcttca

SEQ ID NO: 364 - DNA, 321 bp, Artificial Sequence
  Synthetic
gacatccagatgacccagtctccgtcctccctgtctgcatctgtaggaggcagagtcaccatcacttgccggacaagtcagagcattagcggctatttaaattggtatcagcagaaagcagggaaagcccctaaactcctgatctatgctacatccaatttgcaaagtggggtcccatcaaggttcactggcagtggctctgggacagatttcactctcaccatcagcagtctgcaacctgaagattttgcagcttactactgtcatcagagtcacagtcccccattcactttcggccctgggaccaaagtggctttcaaa

SEQ ID NO: 365 - DNA, 24 bp, Artificial Sequence
  Synthetic
ggattcaccttcagtagttatgaa

SEQ ID NO: 366 - DNA, 24 bp, Artificial Sequence
  Synthetic
attagtagtggtggtagttctata

SEQ ID NO: 367 - DNA, 45 bp, Artificial Sequence
  Synthetic
gcgagagtgggtggaagctactactactcctacgctatggacgtc

SEQ ID NO: 368 - DNA, 18 bp, Artificial Sequence
  Synthetic
caggacattagcagttat

SEQ ID NO: 369 - DNA, 27 bp, Artificial Sequence
  Synthetic
caacagcctcataattccccattcagt

SEQ ID NO: 370 - DNA, 366 bp, Artificial Sequence
  Synthetic
gaggtgcagttggtggagtctgggggaggcttggtccagcctggagggtccctgagactctcctgtgcagcctctggattcaccttcagtagttatgaaatgaactgggtccgccaggctccagggaagggactggagtgggtttcatacattagtagtggtggtagttctataaaatacgcagactctgtgaggggccgattcgccttctccagagacaacgccgagaactcagtgcatctgcaaatgaacagcctgagagccgaggacacggctgtttattactgtgcgagagtgggtggaagctactactactcctacgctatggacgtctggggccaagggaccgcggtcaccgtctcctca

SEQ ID NO: 371 - DNA, 321 bp, Artificial Sequence
  Synthetic
gacatccagttgacccagtctccatccttcctgtctgcatctgtaggagacagagtcaccatcacttgctgggccagtcaggacattagcagttatttagcctggtatcagcaaaaaccagggaaagcccctaagctcctgatctatgctgcatccactttgcaaagtggggtcccatcaaggttcaccggcagtggatctgggacagaattcactctcacaatcagcagcctgcagcctgaagattttgcaacttattactgtcaacagcctcataattccccattcagtttcggccctgggaccaaagtggatgtcaaa

SEQ ID NO: 372 - DNA, 39 bp, Artificial Sequence
  Synthetic
gtgagagagcgtagagatggccacaaaatctttgactat

SEQ ID NO: 373 - DNA, 360 bp, Artificial Sequence
  Synthetic
caggtgcagctggtggagtctgggggagacttggtcaagcctggagggtccctgagactctcctgtgtagcctctggattcaccttcagtgactactacatgaactggatccgcctgcctccagggaagggcctggagtggatttcatacattgatagtagtgctggtgccatttactatgcagactctgtgaagggccgattcaccgtctccagggacgacgccaagaactcactatttctgcacatgaacagcctgagagccgatgacacggccgtttattactgtgtgagagagcgtagagatggccacaaaatctttgactattggggcccgggaaccctggtcaccgtctcctcc

SEQ ID NO: 374 - DNA, 339 bp, Artificial Sequence
  Synthetic
gacatcgtgatgacccagtctccagactccctggctgtgtctctgggcgagagggccaccatcaactgcaagtccagccagaatattttatacagttccaacaataagaactacttagcttggtaccagcagaaagcaggacagcctcctaagttgctcattttctggtcatctattcgggaatccggggtccctgaccgattcagtggcagcgggtctgggacagatttcactctcaccatcagcagcctgcaggctgaagatgtggcagtttattattgtcagcaatattacagtagtccgtataattttggccgggggaccacactggagatcaaa

SEQ ID NO: 375 - DNA, 48 bp, Artificial Sequence
  Synthetic
gggaaagatccatataactggaatcacggggtctacggcatggacgtc

SEQ ID NO: 376 - DNA, 18 bp, Artificial Sequence
  Synthetic
cagagcattagcacctat

SEQ ID NO: 377 - DNA, 9 bp, Artificial Sequence
  Synthetic
ggtgcttcc

SEQ ID NO: 378 - DNA, 27 bp, Artificial Sequence
  Synthetic
caacagagttacaatatccctctcact

SEQ ID NO: 379 - DNA, 369 bp, Artificial Sequence
  Synthetic
gaggtgcagctgttggaatctgggggaggcctgatacaaccgggggggtccctgagactctcctgtgcagcctctggattcacctttagtggcttcgccatgagctgggtccgccaggctcctgggaaggggctggagtgggtctcaggtattagtgggagtggtgatatcacatactatggagactccgtgaagggccggttcaccatttccagagacaattccaagagtacgctgtatctgcaaatgaacagcctgagagacgcggacacggccgtatattattgtgggaaagatccatataactggaatcacggggtctacggcatggacgtctggggccaagggaccacggtcaccgtctcctca

SEQ ID NO: 380 - DNA, 321 bp, Artificial Sequence
  Synthetic
gacatccagatgacccagtctccatcctccctgtctgcatccgtaggagacagagtcaccatcacttgccgggcaagtcagagcattagcacctatttaaattggtatcagcagaagccagggaaagcccctaagttcctgatctatggtgcttccagtttgcaaagtggggtcccatcaaggttcagtggcagtggatctgggacagatttctctctcaccatcagcagtctgcaccctggagacttcgcaacttactactgtcaacagagttacaatatccctctcactttcggcggagggaccaaggtggagatcaaa

SEQ ID NO: 381 - DNA, 24 bp, Artificial Sequence
  Synthetic
ggattcgcctttcacacctatgac

SEQ ID NO: 382 - DNA, 24 bp, Artificial Sequence
  Synthetic
gttgttggtagtggtattaacaca

SEQ ID NO: 383 - DNA, 36 bp, Artificial Sequence
  Synthetic
gcgaaagatagcagcagttggttttccctccactac

SEQ ID NO: 384 - DNA, 18 bp, Artificial Sequence
  Synthetic
cagagcataagtaactgg

SEQ ID NO: 385 - DNA, 9 bp, Artificial Sequence
  Synthetic
aaggcgtct

SEQ ID NO: 386 - DNA, 27 bp, Artificial Sequence
  Synthetic
caacagtatagaagttatgcgtacact

SEQ ID NO: 387 - DNA, 357 bp, Artificial Sequence
  Synthetic
gaggtgcagttgttggagtctgggggaggcttggtacagcctggggggtccctgagactctcctgtgcagcctctggattcgcctttcacacctatgacatgacctgggtccgccaggctccagggaagggactggagtgggtctcaggtgttgttggtagtggtattaacacatactacgcagactccgtgaagggccggttcaccatttccagagacaattccaagagcacgctctatctgcaaatgaacagtctgagagccgaggacacggccgtatattactgtgcgaaagatagcagcagttggttttccctccactactggggccagggaaccctggtcaccgtctcctca

SEQ ID NO: 388 - DNA, 321 bp, Artificial Sequence
  Synthetic
gacatccagatgacccagtctccttccaccctgtctgcatctgtaggagacagagtcaccatcacttgccgggccagtcagagcataagtaactggttggcctggtatcagcagaaaccagggaaagcccctaaactcctgatctataaggcgtctagtttagaaagtggggtcccatcaaggttcagcggcagtggatctgggacagatttcactctcaccatcagcagcctgcagcctgatgattttgcaagttattactgccaacagtatagaagttatgcgtacacttttggccaggggaccaagctggagatcaca

SEQ ID NO: 389 - DNA, 24 bp, Artificial Sequence
  Synthetic
ggattcaccttcagtgactattac

SEQ ID NO: 390 - DNA, 24 bp, Artificial Sequence
  Synthetic
attgatagtagtgctggtgccact

SEQ ID NO: 391 - DNA, 39 bp, Artificial Sequence
  Synthetic
gcgagagagcgtagaagtggccacaaaatctttgactgt

SEQ ID NO: 392 - DNA, 360 bp, Artificial Sequence
  Synthetic
caggtgcagttggtggagtctgggggagacttggtcaagcctggagggtccctgagactctcctgtgtagcctctggattcaccttcagtgactattacatgaactggatccgcctgcctccagggaagggcctggagtggatttcatacattgatagtagtgctggtgccacttactatgcagactctgtgaagggccgattcaccgtctccagggacgacgccaagaattcactatttctgcacatgaacagtctgagagccgaggacacggccgtttattactgtgcgagagagcgtagaagtggccacaaaatctttgactgttggggcccgggaaccctggtcaccgtctcctca

SEQ ID NO: 393 - DNA, 24 bp, Artificial Sequence
  Synthetic
attgatagtagcgctggtgccatt

SEQ ID NO: 394 - DNA, 360 bp, Artificial Sequence
  Synthetic
caggtgcacctggtggagtctgggggagacttggtcaagcctggagggtccctgagactctcctgtgtagcctctggattcaccttcagtgactactacatgaactggatccgcctgcctccagggaagggcctggagtggatttcatacattgatagtagcgctggtgccatttactatgcagactctgtgaagggccgattcaccgtctccagggacgacgccaagaactcaatgtttctgcacatgaacagcctgagagccgatgacacggccgtttattactgtgcgagagagcgtagagatggccacaaaatctttgactattggggcccgggaaccctggtcaccgtctcctca

SEQ ID NO: 395 - DNA, 24 bp, Artificial Sequence
  Synthetic
ggtttcaccttcagtgactactac

SEQ ID NO: 396 - DNA, 39 bp, Artificial Sequence
  Synthetic
gcgagagagcggagagaaggccacaaaatctttgacttt

SEQ ID NO: 397 - DNA, 360 bp, Artificial Sequence
  Synthetic
caggtgcaactggtggagtctgggggagacttggtcaagcctggagggtccctgagactctcctgtgtagcctctggtttcaccttcagtgactactacatgaactggatccgcctgcctccagggaagggcctggagtggatttcatacattgatagtagtgctggtgccatttactatgcagactctgtgaaggggcgattcaccgtctccagggacgacgccaagaactcattatttctgcacatgaacagcctgagagccgatgacacggccgtttattactgtgcgagagagcggagagaaggccacaaaatctttgacttttggggcccgggaaccctggtcaccgtctcctca

SEQ ID NO: 398 - DNA, 339 bp, Artificial Sequence
  Synthetic
gacatcgtgatgacccagtctccagactccctggctctgtctctgggcgagagggccaccattaactgcaagtccagccagaatattttatacagttccaacaataagaactacttagcttgttaccaacagaaagcaggacagcctcctaagttgctcattttctggtcatctacccgggaatccggggtccctgaccgattcagtggcagcgggtctgggacagatttcactctcaccatcagcagcctgcaggctgaagatgtggcagtttattactgtcagcaatattacagtagtccgtataatttcggccgggggaccacactggagatcaaa

SEQ ID NO: 399 - DNA, 24 bp, Artificial Sequence
  Synthetic
ggcttcacctttcacaactatgcc

SEQ ID NO: 400 - DNA, 24 bp, Artificial Sequence
  Synthetic
attagtggtagtggtggtaccgca

SEQ ID NO: 401 - DNA, 42 bp, Artificial Sequence
  Synthetic
gcgaaagaccgatttttggagtgggtagaggggttcgactcc

SEQ ID NO: 402 - DNA, 18 bp, Artificial Sequence
  Synthetic
cagagcattggcagcttt

SEQ ID NO: 403 - DNA, 27 bp, Artificial Sequence
  Synthetic
caacagagttacaataccccgctcact

SEQ ID NO: 404 - DNA, 363 bp, Artificial Sequence
  Synthetic
gaggtgcagctgttggagtctgggggaggcttggtacagcctggggggtccctgagactctcctgtgcagcctctggcttcacctttcacaactatgccatgagctgggtccgccaggctccagggaaggggctggagtgggtctcaggtattagtggtagtggtggtaccgcatactacgcagactccgtgaagggccggttcaccatttccagagacaattccaagaacacgctgtatctgcaaatgaacagcctgagagccgacgacacggccttatattactgtgcgaaagaccgatttttggagtgggtagaggggttcgactcctggggccagggaaccctggtcaccgtctcctca

SEQ ID NO: 405 - DNA, 321 bp, Artificial Sequence
  Synthetic
gacatccagatgacccagtctccatcctccctgtctgcatctgtaggagacagagtcaccatcacttgccgggcaagtcagagcattggcagctttttaatttggtatcagcagaaaccagggaaagcccctaagctcctgatttatgctgcatccagtttgcaaagtggggtcccatcaaggttcagtggcagtggatctgggacagatttcactctcattatcagcagtctgcaacctgaagattttgcaacttattactgtcaacagagttacaataccccgctcactttcggcggagggaccaaagtggagatcaaa

SEQ ID NO: 406 - DNA, 24 bp, Artificial Sequence
  Synthetic
attagtagtggtggtagttccata

SEQ ID NO: 407 - DNA, 27 bp, Artificial Sequence
  Synthetic
caacagcctaataattacccattcagt

SEQ ID NO: 408 - DNA, 366 bp, Artificial Sequence
  Synthetic
gaggtgcagttggtggagtctgggggaggcttggtccagcctggagggtccctgagactctcctgtgcagcctctggattcaccttcagtagttatgaaatgaactgggtccgccaggctccagggaagggactggagtgggtttcatacattagtagtggtggtagttccataaaatacgcagactctgtgaggggccgattcaccttctccagagacaacgccgagaactcagtgcatctgcaaatgaacagcctgagaggcgaggacacggctgtttattactgtgcgagagtgggtggaagctactactactcctacgctatggacgtctggggccaagggaccgcggtcaccgtctcctca

SEQ ID NO: 409 - DNA, 321 bp, Artificial Sequence
  Synthetic
gacatccagttgacccagtctccatccttcctgtctgcatctgtaggagacagagtcaccatcacttgctgggccagtcaggacattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcctaataattacccattcagtttcggccctgggaccaaagtggatgtcaaa

SEQ ID NO: 410 - DNA, 366 bp, Artificial Sequence
  Synthetic
gaggtgcagttggtggagtctgggggaggcttggtccagcctggagggtccctgagactctcctgtgcagcctctggattcaccttcagtagttatgaaatgaactgggtccgccaggctccagggaagggactggagtgggtttcatacattagtagtggtggtagttccataaaatacgcagactctgtgaggggccgattcaccttctccagagacaacgccgagaactcagtgtttctccaaatgaacagcctgagaggcgaggacacggctgtttattactgtgcgagagtgggtggaagctactactactcctacgctatggacgtctggggccaagggaccgcggtcaccgtctcctca

SEQ ID NO: 411 - DNA, 321 bp, Artificial Sequence
  Synthetic
gacatccagttgacccagtctccatccttcctgtctgcatctgtaggagacagagtcaccatcacttgctgggccagtcaggacattagcagttatttagcctggtatcagcaaaaaccagggaaagcccctaagctcctgatctatgctgcatccactttacaaagtggggtcccatcaaggttcagcggcagtggatctgggacagaattcactctcacaatcagcagcctgcagcctgaagattttgcaacttattactgtcaacagcctaataattacccattcagtttcggccctgggaccaaagtggatgtcaaa

SEQ ID NO: 412 - DNA, 24 bp, Artificial Sequence
  Synthetic
attagtgctagtggcggtaccaca

SEQ ID NO: 413 - DNA, 42 bp, Artificial Sequence
  Synthetic
gcgaaagaccgatttttggagtgggtagaggggttcgacccc

SEQ ID NO: 414 - DNA, 18 bp, Artificial Sequence
  Synthetic
cagagcattggcaggttt

SEQ ID NO: 415 - DNA, 27 bp, Artificial Sequence
  Synthetic
caacagagttacattaccccgctcact

SEQ ID NO: 416 - DNA, 363 bp, Artificial Sequence
  Synthetic
gaggtgcagttgttggagtctgggggaggcttggtacagcctggggggtccctgagactctcctgtgcagtctctggattcacctttagcagctttgccatgagctgggtccgccaggctccagggaagggcctggagtgggtctcgggtattagtgctagtggcggtaccacaaattacgcagactccgtgaaaggccggttcaccatctccagagacaattccaagaacacgctctatctgcaaatgagcagcctgagagccgaggacacggccgaatattactgtgcgaaagaccgatttttggagtgggtagaggggttcgacccctggggccagggaatcctggtcaccgtctcccca

SEQ ID NO: 417 - DNA, 321 bp, Artificial Sequence
  Synthetic
gacatccagatgacccagtctccatcctccctgtctgcatctgtaggagacagagtcaccatcacttgccgggcaagtcagagcattggcaggtttttaatttggtatcagcagaaaccagggaaagcccctaaactcctgctttatgctacatccagtttgcaaagtggggtcccagcaaggttcactggcagtgggtctgggacagatttcactctcaccatcggcagtctgcaacctgaagattttgcaacttactactgtcaacagagttacattaccccgctcactttcggcggagggaccaaggtggagatcaaa

SEQ ID NO: 418 - DNA, 363 bp, Artificial Sequence
  Synthetic
gaggtgcagttgttggagtctgggggaggcttggtacagcctggggggtccctgagactctcctgtgcagcctctggattcacctttagcagctttgccatgagctgggtccgccaggctccagggaagggcctggagtgggtctcgggtattagtgctagtggcggtaccacaaattacgcagactccgtgaaaggccggttcaccatctccagagacaattccaagaacacgctctatctgcaaatgagcagcctgagagccgaggacacggccgaatattactgtgcgaaagaccgatttttggagtgggtagaggggttcgacccctggggccagggaatcctggtcaccgtctcccca

SEQ ID NO: 419 - DNA, 321 bp, Artificial Sequence
  Synthetic
gacatccagatgacccagtctccatcctccctgtctgcatctgtaggagacagagtcaccatcacttgccgggcaagtcagagcattggcaggtttttaatttggtatcagcagaaaccagggaaagcccctaagctcctgctctatgctacatccagtttgcaaagtggggtcccagcaaggttcactggcagtgggtctgggacagatttcactctcaccatcggcagtctgcaacctgaagattttgcaacttactactgtcaacagagttacattaccccgctcactttcggcggagggaccaaggtggagatcaaa

SEQ ID NO: 420 - DNA, 75 bp, Artificial Sequence
  Synthetic
caggtgcagctggtggagtctgggggagacttggtcaagcctggagggtccctgagactctcctgtgtagcctcg

SEQ ID NO: 421 - DNA, 51 bp, Artificial Sequence
  Synthetic
atgaactggatccgcctgcctccagggaagggcctggagtgggtttcatac

SEQ ID NO: 422 - DNA, 114 bp, Artificial Sequence
  Synthetic
tactatgcagactctgtgaggggccgattcaccgtctccagggacgacgccaagaattcactatttctgcacatgaacagcctgagagccgatgacacggccgtttattactgt

SEQ ID NO: 423 - DNA, 33 bp, Artificial Sequence
  Synthetic
tggggcccgggaaccctggtcaccgtctcttca

SEQ ID NO: 424 - DNA, 78 bp, Artificial Sequence
  Synthetic
gacatcgtgatgacccagtctccagactccctggctgtgtctctgggcgagagggccaccatcaactgcaagtccagc

SEQ ID NO: 425 - DNA, 51 bp, Artificial Sequence
  Synthetic
ttagcttggtaccagcagaaagcaggacagcctcctaagttgctcattttc

SEQ ID NO: 426 - DNA, 108 bp, Artificial Sequence
  Synthetic
acccgggaatccggggtccctgaccgattcagtggcagcgggtctgggacagatttcactctcaccatcaacagcctgcaggctgaagatgtggcagtttattactgt

SEQ ID NO: 427 - DNA, 30 bp, Artificial Sequence
  Synthetic
tttggccgggggaccacactggagatcaaa

SEQ ID NO: 428 - DNA, 75 bp, Artificial Sequence
  Synthetic
caggtgcacctggtggagtctgggggagacttggtcaagcctggagggtccctgagactctcctgtgtagcctct

SEQ ID NO: 429 - DNA, 51 bp, Artificial Sequence
  Synthetic
atgaactggatccgtatgcctccagggaagggcctggagtggatttcatac

SEQ ID NO: 430 - DNA, 114 bp, Artificial Sequence
  Synthetic
tactatgcagactctgtgaagggccgattcaccgtctccagggacgacgccaagaactcactgtttctacacatgaacaacctgagagccgatgacacggccgtttattactgt

SEQ ID NO: 431 - DNA, 33 bp, Artificial Sequence
  Synthetic
tggggcccgggaaccctggtcaccgtctcctca

SEQ ID NO: 432 - DNA, 108 bp, Artificial Sequence
  Synthetic
acccgggaatccggggtccctgaccgattcagtggcagcgggtctgggacagatttcactctcaccatcagcagcctgcaggctgaagatgtggcagtttattactgt

SEQ ID NO: 433 - DNA, 75 bp, Artificial Sequence
  Synthetic
gaggtgcagctggtgcagtctggagcagaggtgaaaaagcccggggagtctctgaagatctcctgtaagggttct

SEQ ID NO: 434 - DNA, 51 bp, Artificial Sequence
  Synthetic
atcggctgggtgcgccagatgcccgggaaaggcctggagtggatggggatc

SEQ ID NO: 435 - DNA, 114 bp, Artificial Sequence
  Synthetic
agatacagcccgtccttccaaggccaggtcaccatctcagccgacaagtccatcagtaccgcctatttgcagtggagcagcctgaaggcctcggacaccgccatgtattactgt

SEQ ID NO: 436 - DNA, 33 bp, Artificial Sequence
  Synthetic
tggggccagggaaccctggtcaccgtctcctca

SEQ ID NO: 437 - DNA, 78 bp, Artificial Sequence
  Synthetic
gacatccagatgacccagtctccatcctccctgtctgcatctgtcggagacagactcaccatcacttgccgggcaagt

SEQ ID NO: 438 - DNA, 51 bp, Artificial Sequence
  Synthetic
ttaaattggtatcaacagaacccagggaaagcccctaaggtcctgatttat

SEQ ID NO: 439 - DNA, 108 bp, Artificial Sequence
  Synthetic
agtttggaaagtggggtcccatcaaggttcagtggcagtggatctgggacagatttcactctcaccatcagcagtctgcaacctgaagattttgcaacttactactgt

SEQ ID NO: 440 - DNA, 30 bp, Artificial Sequence
  Synthetic
ttcggccaagggaccaaggtggaaatcaaa

SEQ ID NO: 441 - DNA, 75 bp, Artificial Sequence
  Synthetic
gaggtgcagcttttggagtcggggggaggcttggcacagccggggaagtccctgagactctcctgtgcagcctct

SEQ ID NO: 442 - DNA, 51 bp, Artificial Sequence
  Synthetic
atgagctgggtccgccaggctcctgggaaggggctggagtgggtctcaggt

SEQ ID NO: 443 - DNA, 114 bp, Artificial Sequence
  Synthetic
tactatgcagactccgtgaagggccggttcaccatttccagagacaattccaagagtacgctgtatcttcaaatgaacagcctgggagccgaggacacggccgtgtattactgt

SEQ ID NO: 444 - DNA, 33 bp, Artificial Sequence
  Synthetic
tggggccaagggaccacggtcaccgtctcctca

SEQ ID NO: 445 - DNA, 78 bp, Artificial Sequence
  Synthetic
gacatccagatgacccagtctccatcctccctgtctgcatctgtaggagacagagtcaccatcacttgccgggcaagt

SEQ ID NO: 446 - DNA, 51 bp, Artificial Sequence
  Synthetic
ttaaattggtatcagcagaaaccagggaaagcccctaaattcctgatttat

SEQ ID NO: 447 - DNA, 108 bp, Artificial Sequence
  Synthetic
  misc_feature (50)..(50): n is a, c, g, or t
agtttgcaaagtggggtcccatcaaggttcagtggcagtggatgtgggantgatttcactctcaccatcagcagtctgcaacgtgaagatttcgcagcttactactgt

SEQ ID NO: 448 - DNA, 31 bp, Artificial Sequence
  Synthetic
ttcggcggggggaccaaggtggagatcaaac

SEQ ID NO: 449 - DNA, 75 bp, Artificial Sequence
  Synthetic
caggtgcagctggtggagtctgggggaggcttggtcaagcctggagggtccctgagactctcctgcgtagcctct

SEQ ID NO: 450 - DNA, 51 bp, Artificial Sequence
  Synthetic
atgaactggatccgcctgcctccagggagggggctgcagtggatttcatac

SEQ ID NO: 451 - DNA, 114 bp, Artificial Sequence
  Synthetic
tactacgcagactctgtgaagggccgattcaccgtctccagggacgacgccaagaattcactttatctgcaaatgaacagcctgagagccgatgacacggccgtttattactgt

SEQ ID NO: 452 - DNA, 51 bp, Artificial Sequence
  Synthetic
ttaggttggtaccagcagaaagcaggacagcctcctaaactgctcatttac

SEQ ID NO: 453 - DNA, 108 bp, Artificial Sequence
  Synthetic
acccggaattccggggtccctgaccgcttcagtggcagcgggtctgggacagatttcactctcaccatcagcagcctgcaggctgaggatgtggcagtttattactgt

SEQ ID NO: 454 - DNA, 30 bp, Artificial Sequence
  Synthetic
tttggccaggggaccaacctggagatcaaa

SEQ ID NO: 455 - DNA, 114 bp, Artificial Sequence
  Synthetic
agatacagcccgtccttccaaggccaggtcaccctctcagccgacaagtccatcagtaccgcctacctacagtggaacagcctgaaggcctcggacaccgccatgtattactgt

SEQ ID NO: 456 - DNA, 34 bp, Artificial Sequence
  Synthetic
tggggccagggaaccctggtcaccgtcgcctcat

SEQ ID NO: 457 - DNA, 51 bp, Artificial Sequence
  Synthetic
ttaaattggtatcagcagaaaccagggacagcccctaaactcctgatttat

SEQ ID NO: 458 - DNA, 108 bp, Artificial Sequence
  Synthetic
agtttgcaaggtggggtcccatcaaggttcagtggcagtggctctgggacagatttcactctcaccatcagcagtctgcaacctgaagattttgcaacttactactgt

SEQ ID NO: 459 - DNA, 75 bp, Artificial Sequence
  Synthetic
caggtgcagctggtggagtctgggggagacttggtcaagcctggagggtccctgagactctcctgtgtagcctct

SEQ ID NO: 460 - DNA, 51 bp, Artificial Sequence
  Synthetic
atgaactggatccgcctgcctccagggaagggcctggagtggatttcctac

SEQ ID NO: 461 - DNA, 114 bp, Artificial Sequence
  Synthetic
tactatgcagactctgtgaagggccgattcaccgtctccagggacgacgccaagaactcactatttctgcacatgaacagcctgagagccgatgacacggccgtttattactgt

SEQ ID NO: 462 - DNA, 51 bp, Artificial Sequence
  Synthetic
ttagcttggtaccagcagaaaacaggacagcctcctaagttgctcattttc

SEQ ID NO: 463 - DNA, 108 bp, Artificial Sequence
  Synthetic
acccgggaatccggggtccctgaccgattcagtggcagcgggtctgggacagatttcactctcaccatcagcagcctgcaggctgaggatgtggcagtttattactgt

SEQ ID NO: 464 - DNA, 75 bp, Artificial Sequence
  Synthetic
gaagtgcagttggtggagtctgggggagacttggtacagcctggcaggtccctgagactctcctgtgcagcctct

SEQ ID NO: 465 - DNA, 51 bp, Artificial Sequence
  Synthetic
atgcactgggtccggcaaggtccagggaagggcctggagtgggtctcaggc

SEQ ID NO: 466 - DNA, 114 bp, Artificial Sequence
  Synthetic
ggctatgcggactctgtgaagggccgattcaccatttccagagacaacgccaagaactccctgtacctgcgaatgaacagtctgagagctgacgacacggccttctattactgt

SEQ ID NO: 467 - DNA, 33 bp, Artificial Sequence
  Synthetic
tggggccacgggacaatggtcaccgtctcttca

SEQ ID NO: 468 - DNA, 51 bp, Artificial Sequence
  Synthetic
ttggattggtatcagcagaaaccaggacagcctcctaagctgctcatttat

SEQ ID NO: 469 - DNA, 108 bp, Artificial Sequence
  Synthetic
tcccgggaatccggggtccctgaccgattcagtggcagcgggtctgggacagatttcactctgaccatcagcagcctgcaggctgaagatgtggcagtttattactgt

SEQ ID NO: 470 - DNA, 30 bp, Artificial Sequence
  Synthetic
ttcggcggagggaccaaggtggagatcaaa

SEQ ID NO: 471 - DNA, 51 bp, Artificial Sequence
  Synthetic
atgaactggatccgcctgcctccagggaagggcctggagtggatttcatac

SEQ ID NO: 472 - DNA, 114 bp, Artificial Sequence
  Synthetic
tactatgcagactctgtgaagggccgattcaccgtctccagggacgacgccaagaactcactatttctacacatgaacaacctgagagccgatgacacggccgtttattactgt

SEQ ID NO: 473 - DNA, 51 bp, Artificial Sequence
  Synthetic
atgaactggatccgcctgcctccagggaggggcctggagtggatttcatac

SEQ ID NO: 474 - DNA, 114 bp, Artificial Sequence
  Synthetic
tactatgcagactctgtgaagggccgattcaccgtctccagggacgacgccaagaagtcactatttctgtacatgaacagcctgagagccgatgacacggccgtttattactgt

SEQ ID NO: 475 - DNA, 75 bp, Artificial Sequence
  Synthetic
gaggtgcagttgttggagtctgggggaggcttggtacagcctggggggtccctgagactctcctgtgcagcctct

SEQ ID NO: 476 - DNA, 51 bp, Artificial Sequence
  Synthetic
atgagctgggtccgccaggctccagggaaggggctggagtgggtctcaggt

SEQ ID NO: 477 - DNA, 114 bp, Artificial Sequence
  Synthetic
tactacgcagactccgtgaagggccggttcaccatctccagagacaattccaagaacacgctgtatctgcaaatgaacagcctgagagccgaggacacggccatatattactgt

SEQ ID NO: 478 - DNA, 33 bp, Artificial Sequence
  Synthetic
tggggccaagggacgatggtcaccgtctcttca

SEQ ID NO: 479 - DNA, 78 bp, Artificial Sequence
  Synthetic
gacatccagatgacccagtctccgtcctccctgtctgcatctgtaggaggcagagtcaccatcacttgccggacaagt

SEQ ID NO: 480 - DNA, 51 bp, Artificial Sequence
  Synthetic
ttaaattggtatcagcagaaagcagggaaagcccctaaactcctgatctat

SEQ ID NO: 481 - DNA, 108 bp, Artificial Sequence
  Synthetic
aatttgcaaagtggggtcccatcaaggttcactggcagtggctctgggacagatttcactctcaccatcagcagtctgcaacctgaagattttgcagcttactactgt

SEQ ID NO: 482 - DNA, 30 bp, Artificial Sequence
  Synthetic
ttcggccctgggaccaaagtggctttcaaa

SEQ ID NO: 483 - DNA, 75 bp, Artificial Sequence
  Synthetic
gaggtgcagttggtggagtctgggggaggcttggtccagcctggagggtccctgagactctcctgtgcagcctct

SEQ ID NO: 484 - DNA, 51 bp, Artificial Sequence
  Synthetic
atgaactgggtccgccaggctccagggaagggactggagtgggtttcatac

SEQ ID NO: 485 - DNA, 114 bp, Artificial Sequence
  Synthetic
aaatacgcagactctgtgaggggccgattcgccttctccagagacaacgccgagaactcagtgcatctgcaaatgaacagcctgagagccgaggacacggctgtttattactgt

SEQ ID NO: 486 - DNA, 33 bp, Artificial Sequence
  Synthetic
tggggccaagggaccgcggtcaccgtctcctca

SEQ ID NO: 487 - DNA, 78 bp, Artificial Sequence
  Synthetic
gacatccagttgacccagtctccatccttcctgtctgcatctgtaggagacagagtcaccatcacttgctgggccagt

SEQ ID NO: 488 - DNA, 51 bp, Artificial Sequence
  Synthetic
ttagcctggtatcagcaaaaaccagggaaagcccctaagctcctgatctat

SEQ ID NO: 489 - DNA, 108 bp, Artificial Sequence
  Synthetic
actttgcaaagtggggtcccatcaaggttcaccggcagtggatctgggacagaattcactctcacaatcagcagcctgcagcctgaagattttgcaacttattactgt

SEQ ID NO: 490 - DNA, 30 bp, Artificial Sequence
  Synthetic
ttcggccctgggaccaaagtggatgtcaaa

SEQ ID NO: 491 - DNA, 33 bp, Artificial Sequence
  Synthetic
tggggcccgggaaccctggtcaccgtctcctcc

SEQ ID NO: 492 - DNA, 108 bp, Artificial Sequence
  Synthetic
attcgggaatccggggtccctgaccgattcagtggcagcgggtctgggacagatttcactctcaccatcagcagcctgcaggctgaagatgtggcagtttattattgt

SEQ ID NO: 493 - DNA, 75 bp, Artificial Sequence
  Synthetic
gaggtgcagctgttggaatctgggggaggcctgatacaaccgggggggtccctgagactctcctgtgcagcctct

SEQ ID NO: 494 - DNA, 114 bp, Artificial Sequence
  Synthetic
tactatggagactccgtgaagggccggttcaccatttccagagacaattccaagagtacgctgtatctgcaaatgaacagcctgagagacgcggacacggccgtatattattgt

SEQ ID NO: 495 - DNA, 78 bp, Artificial Sequence
  Synthetic
gacatccagatgacccagtctccatcctccctgtctgcatccgtaggagacagagtcaccatcacttgccgggcaagt

SEQ ID NO: 496 - DNA, 51 bp, Artificial Sequence
  Synthetic
ttaaattggtatcagcagaagccagggaaagcccctaagttcctgatttat

SEQ ID NO: 497 - DNA, 108 bp, Artificial Sequence
  Synthetic
  misc_feature (76)..(76): n is a, c, g, or t
agtttgcaaagtggggtcccatcaaggttcagtggcagtggatgtgggacagatttctctctcaccatcagcagtntgcaccctggagacttcgcaacttactactgt

SEQ ID NO: 498 - DNA, 51 bp, Artificial Sequence
  Synthetic
atgacctgggtccgccaggctccagggaagggactggagtgggtctcaggt

SEQ ID NO: 499 - DNA, 114 bp, Artificial Sequence
  Synthetic
tactacgcagactccgtgaagggccggttcaccatttccagagacaattccaagagcacgctctatctgcaaatgaacagtctgagagccgaggacacggccgtatattactgt

SEQ ID NO: 500 - DNA, 78 bp, Artificial Sequence
  Synthetic
gacatccagatgacccagtctccttccaccctgtctgcatctgtaggagacagagtcaccatcacttgccgggccagt

SEQ ID NO: 501 - DNA, 51 bp, Artificial Sequence
  Synthetic
ttggcctggtatcagcagaaaccagggaaagcccctaaactcctgatctat

SEQ ID NO: 502 - DNA, 108 bp, Artificial Sequence
  Synthetic
agtttagaaagtggggtcccatcaaggttcagcggcagtggatctgggacagatttcactctcaccatcagcagcctgcagcctgatgattttgcaagttattactgc

SEQ ID NO: 503 - DNA, 30 bp, Artificial Sequence
  Synthetic
tttggccaggggaccaagctggagatcaca

SEQ ID NO: 504 - DNA, 75 bp, Artificial Sequence
  Synthetic
caggtgcagttggtggagtctgggggagacttggtcaagcctggagggtccctgagactctcctgtgtagcctct

SEQ ID NO: 505 - DNA, 114 bp, Artificial Sequence
  Synthetic
tactatgcagactctgtgaagggccgattcaccgtctccagggacgacgccaagaattcactatttctgcacatgaacagtctgagagccgaggacacggccgtttattactgt

SEQ ID NO: 506 - DNA, 114 bp, Artificial Sequence
  Synthetic
tactatgcagactctgtgaagggccgattcaccgtctccagggacgacgccaagaactcaatgtttctgcacatgaacagcctgagagccgatgacacggccgtttattactgt

SEQ ID NO: 507 - DNA, 75 bp, Artificial Sequence
  Synthetic
caggtgcaactggtggagtctgggggagacttggtcaagcctggagggtccctgagactctcctgtgtagcctct

SEQ ID NO: 508 - DNA, 114 bp, Artificial Sequence
  Synthetic
tactatgcagactctgtgaaggggcgattcaccgtctccagggacgacgccaagaactcattatttctgcacatgaacagcctgagagccgatgacacggccgtttattactgt

SEQ ID NO: 509 - DNA, 78 bp, Artificial Sequence
  Synthetic
gacatcgtgatgacccagtctccagactccctggctctgtctctgggcgagagggccaccattaactgcaagtccagc

SEQ ID NO: 510 - DNA, 51 bp, Artificial Sequence
  Synthetic
ttagcttgttaccaacagaaagcaggacagcctcctaagttgctcattttc

SEQ ID NO: 511 - DNA, 30 bp, Artificial Sequence
  Synthetic
ttcggccgggggaccacactggagatcaaa

SEQ ID NO: 512 - DNA, 75 bp, Artificial Sequence
  Synthetic
gaggtgcagctgttggagtctgggggaggcttggtacagcctggggggtccctgagactctcctgtgcagcctct

SEQ ID NO: 513 - DNA, 114 bp, Artificial Sequence
  Synthetic
tactacgcagactccgtgaagggccggttcaccatttccagagacaattccaagaacacgctgtatctgcaaatgaacagcctgagagccgacgacacggccttatattactgt

SEQ ID NO: 514 - DNA, 51 bp, Artificial Sequence
  Synthetic
ttaatttggtatcagcagaaaccagggaaagcccctaagctcctgatttat

SEQ ID NO: 515 - DNA, 108 bp, Artificial Sequence
  Synthetic
agtttgcaaagtggggtcccatcaaggttcagtggcagtggatctgggacagatttcactctcattatcagcagtctgcaacctgaagattttgcaacttattactgt

SEQ ID NO: 516 - DNA, 30 bp, Artificial Sequence
  Synthetic
ttcggcggagggaccaaagtggagatcaaa

SEQ ID NO: 517 - DNA, 114 bp, Artificial Sequence
  Synthetic
aaatacgcagactctgtgaggggccgattcaccttctccagagacaacgccgagaactcagtgcatctgcaaatgaacagcctgagaggcgaggacacggctgtttattactgt

SEQ ID NO: 518 - DNA, 51 bp, Artificial Sequence
  Synthetic
ttagcctggtatcagcaaaaaccagggaaagcccctaagctcctgatttat

SEQ ID NO: 519 - DNA, 108 bp, Artificial Sequence
  Synthetic
  misc_feature (44)..(44): n is a, c, g, or t
actttgcaaagtggggtcccatcaaggttcagcggcagtggatntgggacagaattcactctcacaatcagcagcttgcagcgtgaagattttgcaacttattactgt

SEQ ID NO: 520 - DNA, 114 bp, Artificial Sequence
  Synthetic
aaatacgcagactctgtgaggggccgattcaccttctccagagacaacgccgagaactcagtgtttctccaaatgaacagcctgagaggcgaggacacggctgtttattactgt

SEQ ID NO: 521 - DNA, 108 bp, Artificial Sequence
  Synthetic
actttacaaagtggggtcccatcaaggttcagcggcagtggatctgggacagaattcactctcacaatcagcagcctgcagcctgaagattttgcaacttattactgt

SEQ ID NO: 522 - DNA, 75 bp, Artificial Sequence
  Synthetic
gaggtgcagttgttggagtctgggggaggcttggtacagcctggggggtccctgagactctcctgtgcagtctct

SEQ ID NO: 523 - DNA, 51 bp, Artificial Sequence
  Synthetic
atgagctgggtccgccaggctccagggaagggcctggagtgggtctcgggt

SEQ ID NO: 524 - DNA, 114 bp, Artificial Sequence
  Synthetic
aattacgcagactccgtgaaaggccggttcaccatctccagagacaattccaagaacacgctctatctgcaaatgagcagcctgagagccgaggacacggccgaatattactgt

SEQ ID NO: 525 - DNA, 33 bp, Artificial Sequence
  Synthetic
tggggccagggaatcctggtcaccgtctcccca

SEQ ID NO: 526 - DNA, 51 bp, Artificial Sequence
  Synthetic
ttaatttggtatcagcagaaaccagggaaagcccctaaactcctgctttat

SEQ ID NO: 527 - DNA, 108 bp, Artificial Sequence
  Synthetic
agtttgcaaagtggggtcccagcaaggttcactggcagtgggtctgggacagatttcactctcaccatcggcagtctgcaacctgaagattttgcaacttactactgt

SEQ ID NO: 528 - DNA, 51 bp, Artificial Sequence
  Synthetic
ttaatttggtatcagcagaaaccagggaaagcccctaagctcctgctctat

SEQ ID NO: 529 - protein, 362 aa, Unknown
  Synthetic
MAPTKRKGECPGAAPKKPKEPVQVPKLLIKGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGEPLEMQGVLMNYRSKYPDGTITPKNPTAQSQVMNTDHKAYLDKNNAYPVECWVPDPSRNENARYFGTFTGGENVPPVLHVTNTATTVLLDEQGVGPLCKADSLYVSAADICGLFTNSSGTQQWRGLARYFKIRLRKRSVKNPYPISFLLSDLINRRTQRVDGQPMYGMESQVEEVRVFDGTERLPGDPDMIRYIDKQGQLQTKML

(19) United States
(12) Patent Application Publication
(10) Pub. No.: US 2025/0304658 A1
(43) Pub. Date: Oct. 2, 2025

(54) NEUTRALIZING MONOCLONAL ANTIBODIES TO BK VIRUS

(71) Applicant: Icahn School of Medicine at Mount Sinai, New York, NY (US)

(72) Inventors: Thomas M. Moran, New York, NY (US); Thomas A. Kraus, New York, NY (US); James A. Duty, New York, NY (US); Domenico Tortorella, New York, NY (US)

(21) Appl. No.: 18/549,974

(22) PCT Filed: Mar. 11, 2022
(86) PCT No.: PCT/US2022/020050

§ 371 Date: Sep. 11, 2023

Related U.S. Application Data
(60) Provisional application No. 63/160,678, filed on Mar. 12, 2021.

(51) Int. Cl. C07K 16/08; A61K 31/343; A61K 31/366; A61K 31/52; A61K 31/5377; A61K 38/13; A61K 39/00; A61P 31/20

(52) U.S. Cl. CPC C07K 16/084; A61K 31/343; A61K 31/366; A61K 31/52; A61K 31/5377; A61K 38/13; A61P 31/20; A61K 2039/54; C07K 2317/41; C07K 2317/52; C07K 2317/565; C07K 2317/567; C07K 2317/76

(57) ABSTRACT

Provided herein are monoclonal antibodies targeting VP1 of polyomaviruses including BK virus. Also provided are methods of treating or preventing polyomavirus infections using one or more antibodies.

### CROSS REFERENCE TO RELATED APPLICATIONS

This application claims the benefit under 35 U.S.C. § 119(e) of U.S. Provisional Application No. 63/160,678, filed Mar. 12, 2021, entitled “NEUTRALIZING MONOCLONAL ANTIBODIES TO BK VIRUS,” the entire disclosure of which is hereby incorporated by reference in their entireties.

### SEQUENCE LISTING SUBMITTED AS ASCII TEXT FILE VIA EFS-WEB

The present Application contains a Sequence Listing which has been submitted in ASCII format via EFS-Web and is hereby incorporated by reference in its entirety. The ASCII version, created on Jan. 30, 2024, is named SeqList-084284-00283 and is 237,612 bytes in size. By this statement, the Sequence Listing constitutes a part of the instant Specification.

### BACKGROUND OF THE INVENTION

BK virus (BKV) is a dsDNA polyomavirus that is the causative agent of polyomavirus-associated nephropathy (PyVAN) and polyomavirus-associated hemorrhagic cystitis (PyVHC) following kidney transplantation and allogeneic hematopoietic stem cell transplantation, respectively. BK virus consists of genetically distinct genotypes that can give rise to differences in serotypes. Both PyVAN and PyVHC occur almost exclusively in immunosuppressed patients and at present the only treatment is a reduction in immunosuppression that dramatically increases the risk of graft failures. New approaches for treating BK viral disease would be a welcome advance in the art, including in the areas of cancer therapy and organ transplantation.

### SUMMARY OF THE INVENTION

The present disclosure provides, in some aspects, isolated antibodies and antigen binding fragments for treating BK viral disease. In particular aspects and without being bound by theory, the isolated antibodies and antigen binding fragments disclosed herein neutralize BK virus by binding to the capsid protein VP1, thereby inhibiting its interaction with sialic acid moieties on host cells and preventing viral infection. BK virus infection is common, but self-limiting, in healthy individuals, with infected individuals producing antibodies that limit viral replication. When individuals become immunocompromised, such as after the development of conditions such as AIDS, or treatment with immunosuppressive agents, such as in the course of cancer treatment or following organ transplant, loss of antibodies or normal immune system function allows BK virus to replicate to a greater extent. This uncontrolled BK virus replication causes multiple adverse effects, including cystitis, nephropathy, urethral stenosis, and colitis. Inhibiting BK virus replication using monoclonal antibodies thus presents a useful method of preventing viral pathogenesis in immunocompromised individuals and those receiving immunosuppressive therapies.

Furthermore, four distinct serotypes of BK virus have been identified, and transplantation of a kidney containing one serotype of BK virus into a recipient without antibodies specific to that serotype is likely to result in transplant rejection. Thus, identifying the BK viral serotype(s) present in a donated kidney, and administering antibodies specific to that serotype(s) to the prospective recipient before, during, and/or after transplantation, serves as one method of reducing the risk of transplant rejection. Additionally, some antibodies or combinations of antibodies provided herein neutralize more than a single serotype of BK virus, including up to all four serotypes of BK virus, and such antibodies or combinations of antibodies are thus useful for preventing or limiting BK virus replication even when the serotype of BK virus infection is not known.

In some aspects, the present disclosure provides an isolated antibody or antigen binding fragment thereof, comprising, a heavy chain variable region (VH) having three complementarity determining regions (CDRs) of HCDR1, HCDR2, and HCDR3, and a light chain variable region (VL) having three CDRs of LCDR1, LCDR2, and LCDR3, wherein:
(a) HCDR1 comprises at least 90% sequence identity to SEQ ID NO: 1, 9, 17, 25, 33, 41, 49, 57, 65, 73, 81, 89, 97, 105, 113, 121, 129, 137, 145, 153, 161, 169, or 177;
(b) HCDR2 comprises at least 90% sequence identity to SEQ ID NO: 2, 10, 18, 26, 34, 42, 50, 58, 66, 74, 82, 90, 98, 106, 114, 122, 130, 138, 146, 154, 162, 170, or 178;
(c) HCDR3 comprises at least 90% sequence identity to SEQ ID NO: 3, 11, 19, 27, 35, 43, 51, 59, 67, 75, 83, 91, 99, 107, 115, 123, 131, 139, 147, 155, 163, 171, or 179;
(d) LCDR1 comprises at least 90% sequence identity to SEQ ID NO: 4, 12, 20, 28, 36, 44, 52, 60, 68, 76, 84, 92, 100, 108, 116, 124, 132, 140, 148, 156, 164, 172, or 180;
(e) LCDR2 comprises at least 90% sequence identity to SEQ ID NO: 5, 13, 21, 29, 37, 45, 53, 61, 69, 77, 85, 93, 101, 109, 117, 125, 133, 141, 149, 157, 165, 173, or 181; and
(f) LCDR3 comprises at least 90% sequence identity to SEQ ID NO: 6, 14, 22, 30, 38, 46, 54, 62, 70, 78, 86, 94, 102, 110, 118, 126, 134, 142, 150, 158, 166, 174, or 182.

In some aspects, the present disclosure provides a composition comprising two more antibodies, wherein a second antibody comprises at least one CDR sequence which is distinct from at least one CDR sequence of a first antibody.

In some embodiments, the isolated antibody or antigen binding fragments provided herein comprise the following sets of CDR sequences:
(a) HCDR1 comprises SEQ ID NO: 1 or a sequence having at least 90% sequence therewith, HCDR2 comprises SEQ ID NO: 2 or a sequence having at least 90% sequence therewith, HCDR3 comprises SEQ ID NO: 3 or a sequence having at least 90% sequence therewith, LCDR1 comprises SEQ ID NO: 4 or a sequence having at least 90% sequence therewith, LCDR2 comprises SEQ ID NO: 5 or a sequence having at least 90% sequence therewith, and LCDR3 comprises SEQ ID NO: 6 or a sequence having at least 90% sequence therewith;
(b) HCDR1 comprises SEQ ID NO: 9 or a sequence having at least 90% sequence therewith, HCDR2 comprises SEQ ID NO: 10 or a sequence having at least 90% sequence therewith, HCDR3 comprises SEQ ID NO: 11 or a sequence having at least 90% sequence therewith, LCDR1 comprises SEQ ID NO: 12 or a sequence having at least 90% sequence therewith, LCDR2 comprises SEQ ID NO: 13 or a sequence having at least 90% sequence therewith, and LCDR3 comprises SEQ ID NO: 14 or a sequence having at least 90% sequence therewith;
(c) HCDR1 comprises SEQ ID NO: 17 or a sequence having at least 90% sequence therewith, HCDR2 comprises SEQ ID NO: 18 or a sequence having at least 90% sequence therewith, HCDR3 comprises SEQ ID NO: 19 or a sequence having at least 90% sequence therewith, LCDR1 comprises SEQ ID NO: 20 or a sequence having at least 90% sequence therewith, LCDR2 comprises SEQ ID NO: 21 or a sequence having at least 90% sequence therewith, and LCDR3 comprises SEQ ID NO: 22 or a sequence having at least 90% sequence therewith;
(d) HCDR1 comprises SEQ ID NO: 25 or a sequence having at least 90% sequence therewith, HCDR2 comprises SEQ ID NO: 26 or a sequence having at least 90% sequence therewith, HCDR3 comprises SEQ ID NO: 27 or a sequence having at least 90% sequence therewith, LCDR1 comprises SEQ ID NO: 28 or a sequence having at least 90% sequence therewith, LCDR2 comprises SEQ ID NO: 29 or a sequence having at least 90% sequence therewith, and LCDR3 comprises SEQ ID NO: 30 or a sequence having at least 90% sequence therewith;
(e) HCDR1 comprises SEQ ID NO: 33 or a sequence having at least 90% sequence therewith, HCDR2 comprises SEQ ID NO: 34 or a sequence having at least 90% sequence therewith, HCDR3 comprises SEQ ID NO: 35 or a sequence having at least 90% sequence therewith, LCDR1 comprises SEQ ID NO: 36 or a sequence having at least 90% sequence therewith, LCDR2 comprises SEQ ID NO: 37 or a sequence having at least 90% sequence therewith, and LCDR3 comprises SEQ ID NO: 38 or a sequence having at least 90% sequence therewith;
(f) HCDR1 comprises SEQ ID NO: 41 or a sequence having at least 90% sequence therewith, HCDR2 comprises SEQ ID NO: 42 or a sequence having at least 90% sequence therewith, HCDR3 comprises SEQ ID NO: 43 or a sequence having at least 90% sequence therewith, LCDR1 comprises SEQ ID NO: 44 or a sequence having at least 90% sequence therewith, LCDR2 comprises SEQ ID NO: 45 or a sequence having at least 90% sequence therewith, and LCDR3 comprises SEQ ID NO: 46 or a sequence having at least 90% sequence therewith;
(g) HCDR1 comprises SEQ ID NO: 49 or a sequence having at least 90% sequence therewith, HCDR2 comprises SEQ ID NO: 50 or a sequence having at least 90% sequence therewith, HCDR3 comprises SEQ ID NO: 51 or a sequence having at least 90% sequence therewith, LCDR1 comprises SEQ ID NO: 52 or a sequence having at least 90% sequence therewith, LCDR2 comprises SEQ ID NO: 53 or a sequence having at least 90% sequence therewith, and LCDR3 comprises SEQ ID NO: 54 or a sequence having at least 90% sequence therewith;
(h) HCDR1 comprises SEQ ID NO: 65 or a sequence having at least 90% sequence therewith, HCDR2 comprises SEQ ID NO: 66 or a sequence having at least 90% sequence therewith, HCDR3 comprises SEQ ID NO: 67 or a sequence having at least 90% sequence therewith, LCDR1 comprises SEQ ID NO: 68 or a sequence having at least 90% sequence therewith, LCDR2 comprises SEQ ID NO: 69 or a sequence having at least 90% sequence therewith, and LCDR3 comprises SEQ ID NO: 70 or a sequence having at least 90% sequence therewith;
(i) HCDR1 comprises SEQ ID NO: 73 or a sequence having at least 90% sequence therewith, HCDR2 comprises SEQ ID NO: 74 or a sequence having at least 90% sequence therewith, HCDR3 comprises SEQ ID NO: 75 or a sequence having at least 90% sequence therewith, LCDR1 comprises SEQ ID NO: 76 or a sequence having at least 90% sequence therewith, LCDR2 comprises SEQ ID NO: 77 or a sequence having at least 90% sequence therewith, and LCDR3 comprises SEQ ID NO: 79 or a sequence having at least 90% sequence therewith;
(j) HCDR1 comprises SEQ ID NO: 81 or a sequence having at least 90% sequence therewith, HCDR2 comprises SEQ ID NO: 82 or a sequence having at least 90% sequence therewith, HCDR3 comprises SEQ ID NO: 83 or a sequence having at least 90% sequence therewith, LCDR1 comprises SEQ ID NO: 84 or a sequence having at least 90% sequence therewith, LCDR2 comprises SEQ ID NO: 85 or a sequence having at least 90% sequence therewith, and LCDR3 comprises SEQ ID NO: 86 or a sequence having at least 90% sequence therewith;
(k) HCDR1 comprises SEQ ID NO: 89 or a sequence having at least 90% sequence therewith, HCDR2 comprises SEQ ID NO: 90 or a sequence having at least 90% sequence therewith, HCDR3 comprises SEQ ID NO: 91 or a sequence having at least 90% sequence therewith, LCDR1 comprises SEQ ID NO: 92 or a sequence having at least 90% sequence therewith, LCDR2 comprises SEQ ID NO: 93 or a sequence having at least 90% sequence therewith, and LCDR3 comprises SEQ ID NO: 94 or a sequence having at least 90% sequence therewith;
(l) HCDR1 comprises SEQ ID NO: 97 or a sequence having at least 90% sequence therewith, HCDR2 comprises SEQ ID NO: 98 or a sequence having at least 90% sequence therewith, HCDR3 comprises SEQ ID NO: 99 or a sequence having at least 90% sequence therewith, LCDR1 comprises SEQ ID NO: 100 or a sequence having at least 90% sequence therewith, LCDR2 comprises SEQ ID NO: 101 or a sequence having at least 90% sequence therewith, and LCDR3 comprises SEQ ID NO: 102 or a sequence having at least 90% sequence therewith;
(m) HCDR1 comprises SEQ ID NO: 105 or a sequence having at least 90% sequence therewith, HCDR2 comprises SEQ ID NO: 106 or a sequence having at least 90% sequence therewith, HCDR3 comprises SEQ ID NO: 107 or a sequence having at least 90% sequence therewith, LCDR1 comprises SEQ ID NO: 108 or a sequence having at least 90% sequence therewith, LCDR2 comprises SEQ ID NO: 109 or a sequence having at least 90% sequence therewith, and LCDR3 comprises SEQ ID NO: 110 or a sequence having at least 90% sequence therewith;
(n) HCDR1 comprises SEQ ID NO: 113 or a sequence having at least 90% sequence therewith, HCDR2 comprises SEQ ID NO: 114 or a sequence having at least 90% sequence therewith, HCDR3 comprises SEQ ID NO: 115 or a sequence having at least 90% sequence therewith, LCDR1 comprises SEQ ID NO: 116 or a sequence having at least 90% sequence therewith, LCDR2 comprises SEQ ID NO: 117 or a sequence having at least 90% sequence therewith, and LCDR3 comprises SEQ ID NO: 118 or a sequence having at least 90% sequence therewith;
(o) HCDR1 comprises SEQ ID NO: 121 or a sequence having at least 90% sequence therewith, HCDR2 comprises SEQ ID NO: 122 or a sequence having at least 90% sequence therewith, HCDR3 comprises SEQ ID NO: 123 or a sequence having at least 90% sequence therewith, LCDR1 comprises SEQ ID NO: 124 or a sequence having at least 90% sequence therewith, LCDR2 comprises SEQ ID NO: 125 or a sequence having at least 90% sequence therewith, and LCDR3 comprises SEQ ID NO: 126 or a sequence having at least 90% sequence therewith;
(p) HCDR1 comprises SEQ ID NO: 129 or a sequence having at least 90% sequence therewith, HCDR2 comprises SEQ ID NO: 130 or a sequence having at least 90% sequence therewith, HCDR3 comprises SEQ ID NO: 131 or a sequence having at least 90% sequence therewith, LCDR1 comprises SEQ ID NO: 132 or a sequence having at least 90% sequence therewith, LCDR2 comprises SEQ ID NO: 133 or a sequence having at least 90% sequence therewith, and LCDR3 comprises SEQ ID NO: 134 or a sequence having at least 90% sequence therewith;
(q) HCDR1 comprises SEQ ID NO: 137 or a sequence having at least 90% sequence therewith, HCDR2 comprises SEQ ID NO: 138 or a sequence having at least 90% sequence therewith, HCDR3 comprises SEQ ID NO: 139 or a sequence having at least 90% sequence therewith, LCDR1 comprises SEQ ID NO: 140 or a sequence having at least 90% sequence therewith, LCDR2 comprises SEQ ID NO: 141 or a sequence having at least 90% sequence therewith, and LCDR3 comprises SEQ ID NO: 142 or a sequence having at least 90% sequence therewith;
(r) HCDR1 comprises SEQ ID NO: 145 or a sequence having at least 90% sequence therewith, HCDR2 comprises SEQ ID NO: 146 or a sequence having at least 90% sequence therewith, HCDR3 comprises SEQ ID NO: 147 or a sequence having at least 90% sequence therewith, LCDR1 comprises SEQ ID NO: 148 or a sequence having at least 90% sequence therewith, LCDR2 comprises SEQ ID NO: 149 or a sequence having at least 90% sequence therewith, and LCDR3 comprises SEQ ID NO: 150 or a sequence having at least 90% sequence therewith;
(s) HCDR1 comprises SEQ ID NO: 153 or a sequence having at least 90% sequence therewith, HCDR2 comprises SEQ ID NO: 154 or a sequence having at least 90% sequence therewith, HCDR3 comprises SEQ ID NO: 155 or a sequence having at least 90% sequence therewith, LCDR1 comprises SEQ ID NO: 156 or a sequence having at least 90% sequence therewith, LCDR2 comprises SEQ ID NO: 157 or a sequence having at least 90% sequence therewith, and LCDR3 comprises SEQ ID NO: 158 or a sequence having at least 90% sequence therewith;
(t) HCDR1 comprises SEQ ID NO: 161 or a sequence having at least 90% sequence therewith, HCDR2 comprises SEQ ID NO: 162 or a sequence having at least 90% sequence therewith, HCDR3 comprises SEQ ID NO: 163 or a sequence having at least 90% sequence therewith, LCDR1 comprises SEQ ID NO: 164 or a sequence having at least 90% sequence therewith, LCDR2 comprises SEQ ID NO: 165 or a sequence having at least 90% sequence therewith, and LCDR3 comprises SEQ ID NO: 166 or a sequence having at least 90% sequence therewith;
(u) HCDR1 comprises SEQ ID NO: 169 or a sequence having at least 90% sequence therewith, HCDR2 comprises SEQ ID NO: 170 or a sequence having at least 90% sequence therewith, HCDR3 comprises SEQ ID NO: 171 or a sequence having at least 90% sequence therewith, LCDR1 comprises SEQ ID NO: 172 or a sequence having at least 90% sequence therewith, LCDR2 comprises SEQ ID NO: 173 or a sequence having at least 90% sequence therewith, and LCDR3 comprises SEQ ID NO: 174 or a sequence having at least 90% sequence therewith; or
(v) HCDR1 comprises SEQ ID NO: 177 or a sequence having at least 90% sequence therewith, HCDR2 comprises SEQ ID NO: 178 or a sequence having at least 90% sequence therewith, HCDR3 comprises SEQ ID NO: 179 or a sequence having at least 90% sequence therewith, LCDR1 comprises SEQ ID NO: 180 or a sequence having at least 90% sequence therewith, LCDR2 comprises SEQ ID NO: 181 or a sequence having at least 90% sequence therewith, and LCDR3 comprises SEQ ID NO: 182 or a sequence having at least 90% sequence therewith.

In some aspects, the present disclosure provides a composition comprising two more antibodies, wherein a first antibody comprises any of the antibodies (a)-(v) according to the preceding paragraph, and wherein a second antibody does not consist of the first antibody.

In some embodiments, wherein a first antibody comprises any of the antibodies (a)-(v) according to the above paragraph, and wherein a second antibody comprises any of the antibodies (a)-(v) and does not consist of the first antibody.

In some aspects, the present disclosure provides an isolated antibody or antigen binding fragment thereof, comprising, a heavy chain variable region (VH) and a light chain variable region (VL), wherein:
(a) VH comprises at least 90% sequence identity to SEQ ID NO: 7, 15, 23, 31, 39, 47, 55, 63, 71, 79, 87, 95, 103, 111, 119, 127, 135, 143, 151, 159, 167, 175, or 183; and
(b) VL comprises at least 90% sequence identity to SEQ ID NO: 8, 16, 24, 32, 40, 48, 56, 64, 72, 80, 88, 96, 104, 112, 120, 128, 136, 144, 152, 160, 168, 176, or 184.

In some aspects, the present disclosure provides a composition comprising two or more antibodies as described in the preceding paragraph, wherein a second antibody comprises (a) at least one VH sequence which is distinct from at least one VH sequence of a first antibody, or (b) at least one VL sequence which is distinct from at least one VL sequence of a first antibody.

In some embodiments of the isolated antibody or antigen binding fragment provided herein:
(aa) VH comprises SEQ ID NO: 7, and VL comprises SEQ ID NO: 8;
(bb) VH comprises SEQ ID NO: 15, and VL comprises SEQ ID NO: 16;
(cc) VH comprises SEQ ID NO: 31, and VL comprises SEQ ID NO: 32;
(dd) VH comprises SEQ ID NO: 39, and VL comprises SEQ ID NO: 40;
(ee) VH comprises SEQ ID NO: 47, and VL comprises SEQ ID NO: 48;
(ff) VH comprises SEQ ID NO: 55, and VL comprises SEQ ID NO: 56;
(gg) VH comprises SEQ ID NO: 63, and VL comprises SEQ ID NO: 64;
(hh) VH comprises SEQ ID NO: 71, and VL comprises SEQ ID NO: 72;
(ii) VH comprises SEQ ID NO: 79, and VL comprises SEQ ID NO: 80;
(jj) VH comprises SEQ ID NO: 87, and VL comprises SEQ ID NO: 88;
(kk) VH comprises SEQ ID NO: 95, and VL comprises SEQ ID NO: 96;
(ll) VH comprises SEQ ID NO: 103, and VL comprises SEQ ID NO: 104;
(mm) VH comprises SEQ ID NO: 111, and VL comprises SEQ ID NO: 112;
(nn) VH comprises SEQ ID NO: 119, and VL comprises SEQ ID NO: 120;
(oo) VH comprises SEQ ID NO: 127, and VL comprises SEQ ID NO: 128;
(pp) VH comprises SEQ ID NO: 135, and VL comprises SEQ ID NO: 136;
(qq) VH comprises SEQ ID NO: 143, and VL comprises SEQ ID NO: 144;
(rr) VH comprises SEQ ID NO: 151, and VL comprises SEQ ID NO: 152;
(ss) VH comprises SEQ ID NO: 159, and VL comprises SEQ ID NO: 160;
(tt) VH comprises SEQ ID NO: 167, and VL comprises SEQ ID NO: 168;
(uu) VH comprises SEQ ID NO: 175, and VL comprises SEQ ID NO: 176; or
(vv) VH comprises SEQ ID NO: 183, and VL comprises SEQ ID NO: 184.

In some aspects, the present disclosure provides a composition comprising two more antibodies, wherein a first antibody comprises any of the antibodies (aa)-(vv) according to the preceding paragraph, and wherein a second antibody does not consist of the first antibody.

In some embodiments, wherein a first antibody comprises any of the antibodies (aa)-(vv) according to the above paragraph, and wherein a second antibody comprises any of the antibodies (aa)-(vv) and does not consist of the first antibody.

In some aspects, the present disclosure provides an isolated antibody or antigen binding fragment thereof, wherein, when bound to BK virus VP1 protein, the antibody binds to at least one of the following residues: S80, D82, R83, or R170 of SEQ ID NO: 529 or to a corresponding residue of an amino acid sequence having at least 85% sequence identity with SEQ ID NO: 529, and wherein the antibody inhibits or otherwise blocks binding of BK virus VP1 to sialic acid. In various embodiments, the isolated antibody or antigen binding fragment inhibits BK virus from binding to sialic acid by at least 1%, at least 2%, at least 4%, at least 8%, at least 10%, at least 20%, at least 30%, at least 40%, at least 50%, at least 60%, at least 60%, at least 70%, at least 80%, at least 90%, at least 95%, at least 99%, or up to 100% as compared to the level of binding of BK virus to sialic acid in the absence of the isolated antibody or antigen binding fragment.

In some aspects, the present disclosure provides a composition comprising two more antibodies, wherein a first antibody comprises any of the antibodies according to any of the preceding paragraphs, and wherein a second antibody does not consist of the first antibody.

In some embodiments, wherein a first antibody comprises any of the antibodies according to the above paragraphs, and wherein a second antibody comprises any of the antibodies according to the above paragraph and does not consist of the first antibody.

In some aspects, the present disclosure provides an isolated antibody or antigen binding fragment thereof, wherein (a) the antibody is capable of inhibiting infection by a virus comprising VP1 of BK virus serotype 1, a virus comprising VP1 of BK virus serotype 2, a virus comprising VP1 of BK virus serotype 3, and a virus comprising VP1 of BK virus serotype 4, wherein an antibody capable of inhibiting infection by a virus is an antibody that inhibits infection of a target cell by a virus, as determined by a method comprising the steps of:
(i) contacting the virus with the antibody or a control protein; (ii) incubating the virus with target cells; and (iii) determining the proportion of the target cells infected by the virus, or the amount of viral RNA or DNA present in the target cells, at the end of step (ii); wherein determination of a lesser proportion of target cells infected by the virus, or a lesser amount of pseudoviral RNA or DNA present in the target cells, in the presence of the antibody, relative to the amount of target cells infected or amount of viral RNA or DNA in the absence of the antibody, is indicative of inhibition of infection by the virus; and (b) the antibody is capable of binding to an epitope that is present on VP1 of BK virus serotype 1, VP1 of BK virus serotype 2, VP1 of BK virus serotype 3, and VP1 of BK virus serotype 4.

In some aspects, the present disclosure provides a composition comprises two more antibodies, wherein a first antibody comprises any of the antibodies according to the above paragraphs (e.g., the antibodies designated (a)-(v) or (aa) to (vv)), and wherein a second antibody does not consist of the first antibody.

In some embodiments, wherein a first antibody comprises any of the antibodies according to the above paragraphs (e.g., the antibodies designated (a)-(v) or (aa) to (vv)), and wherein a second antibody comprises any of the antibodies according to the above paragraphs and does not consist of the first antibody.

In some embodiments of the antibody or antigen binding fragment provided herein, the antibody has a dissociation constant (K_{D}) from BK virus VP1 protein selected from the group consisting of at most about 10⁻⁷ M; at most about 10⁻⁸ M; at most about 10⁻⁹ M; at most about 10⁻¹⁰ M; at most about 10⁻¹¹ M; at most about 10⁻¹² M; and at most 10⁻¹³ M.

In some embodiments, the antibody or antigen binding fragment further comprises: heavy chain framework regions HFR1, HFR2, HFR3, and HFR4; and light chain framework regions LFR1, LFR2, LFR3, and LFR4.

In some embodiments, the HFR1 region comprises the amino acid sequence of any one of SEQ ID NOs: 185, 193, 197, 205, 213, 226, 234, 241, 250, or 269, the HFR2 region comprises the amino acid sequence of any one of SEQ ID NOs: 186, 194, 198, 206, 214, 223, 227, 232, 242, or 253, the HFR3 region comprises the amino acid sequence of any one of SEQ ID NOs: 187, 195, 199, 207, 215, 219, 224, 228, 233, 235, 243, 251, 254, 258, 259, 262, 265, 267, or 270, the HFR4 region comprises the amino acid sequence of any one of SEQ ID NOs: 188, 200, 208, 220, 229, 236, 244, or 271, the LFR1 region comprises the amino acid sequence of any one of SEQ ID NOs: 189, 201, 209, 237, 245, 255, or 260, the LFR2 region comprises the amino acid sequence of any one of SEQ ID NOs: 190, 202, 210, 216, 221, 225, 230, 238, 246, 261, 263, or 272, the LFR3 region comprises SEQ ID NOs: 191, 196, 203, 211, 217, 222, 230, 239, 247, 249, 252, 256, 264, 266, 268, or 273, and the LFR4 region comprises the amino acid sequence of any one of SEQ ID NOs: 192, 204, 212, 218, 240, 248, or 257.

In some embodiments, the antibody or antigen binding fragment further comprises a heavy chain constant (CH) domain and a light chain constant (CL) domain, wherein:
(a) the CH domain is selected from the group consisting of IgG, IgG1, IgG2, IgG2a, IgG2b, IgG2c, IgG3, IgG4, IgA, IgA1, IgA2, IgD, IgM, and IgE constant domains, and comprises a sequence having at least 80%, 85%, 90%, 95%, 96%, 97%, 98%, 99%, or up to 100% sequence identity with one of SEQ ID NOs: 274-290; and
(b) the CL domain is selected from the group consisting of Ig kappa and Ig lambda constant domains, and comprises a sequence having at least 80%, 85%, 90%, 95%, 96%, 97%, 98%, 99%, or up to 100% sequence identity with one of SEQ ID NOs: 291-301.

In some embodiments, the antibody is a monoclonal antibody, a chimeric antibody, a humanized antibody, a human engineered antibody, a human antibody, a single chain antibody (scFv), or an antibody fragment.

In some embodiments, the antibody has reduced glycosylation, no glycosylation, or is hypofucosylated.

In some aspects, the present disclosure provides a pharmaceutical composition comprising an antibody or antigen binding fragment and a pharmaceutically acceptable excipient. In some embodiments, the pharmaceutically acceptable carrier contains histidine or a sugar. In some embodiments, the sugar is sucrose.

In some aspects, the present disclosure provides a composition comprising a plurality of antibodies or antigen binding fragments, wherein least 0.05%, 0.1%, 0.5%, 1%, 2%, 3%, 5%, or more of the antibodies in the composition have an alpha 2,3-linked sialic acid residue.

In some aspects, the present disclosure provides a composition comprising a plurality of antibodies or antigen binding fragments, wherein none of the antibodies comprise a bisecting Glc-NAc.

In embodiments, the pharmaceutical composition is prepared as a lyophilisate.

In some aspects, the present disclosure provides a method of neutralizing a BK virus infection comprising administering an effective amount of an antibody or composition comprising an antibody to a subject in need thereof. In some embodiments, the subject in need is diagnosed with BK virus infection or BK viremia.

In some embodiments, the present disclosure provides a method of treating or reducing the likelihood of a BK virus or JC virus associated disorder, comprising administering an effective amount of the antibody or the composition comprising an antibody to a subject in need thereof, and wherein the disorder is: transplant rejection, graft-versus-host disease (GvHD), nephropathy, BKVAN, hemorrhagic cystitis (HC), Progressive Multifocal Leukoencephalopathy (PML), granule cell neuronopathy (GCN), interstitial kidney disease, ureteral stenosis, vasculitis, colitis, retinitis, meningitis, immune reconstitution inflammatory syndrome (IRIS).

In some embodiments, the antibody is administered via injection or infusion. In some embodiments, the antibody or composition is reconstituted prior to injection or infusion.

These and other aspects and embodiments will be described in greater detail herein. The description of some exemplary embodiments of the disclosure are provided for illustration purposes only and not meant to be limiting. Additional compositions and methods are also embraced by this disclosure.

In some embodiments, the therapeutic agent is an immunosuppressive agent. In some embodiments, the immunosuppressive agent is a monophosphate dehydrogenase inhibitor, a purine synthesis inhibitor, a calcineurin inhibitor, or an mTOR inhibitor. In some embodiments, the immunosuppressive agent is mycophenolate mofetil (MMF), mycophenolate sodium, azathioprine, tacrolimus, sirolimus, or cyclosporine.

In some embodiments, the therapeutic agent is an additional anti-VP1 antibody.

In some aspects, the present disclosure provides a nucleic acid encoding an antibody or antigen binding fragment described in the above paragraphs.

In some aspects, the present disclosure provides a vector comprising the nucleic acid.

In some aspects the present disclosure provides an isolated host cell comprising the vector.

In some aspects, the present disclosure provides a process for producing an antibody or antigen binding fragment comprising cultivating the host cell and recovering the antibody from the culture.

In some aspects, the present disclosure provides a diagnostic reagent comprising an antibody or antigen binding fragment, which is labeled. In some embodiments, the label is selected from the group consisting of a radiolabel, a fluorophore, a chromophore, an imaging agent, and a metal ion.

In some aspects, the present disclosure provides method of reducing the risk of transplant rejection in a transplant recipient receiving a donor organ, comprising (i) determining serotype of BK virus present in the donor organ, (ii) selecting an antibody or composition comprising an antibody that neutralizes the serotype of BK virus present in the donor organ, and (iii) administering the selected antibody to the transplant recipient via injection or infusion.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF THE DRAWINGS

FIG. 1: Generation, isolation, and evaluation of monoclonal antibodies to BK virus. DNA encoding BK virus VP1, VP2, and VP3 was administered to mice as a primary immunization, then pseudoviruses containing BK virus VP1, VP2, and VP3 were administered to mice as a booster dose. Splenocytes were fused with Sp2/0 cells to form hybridomas, which were cultured to produce antibodies, and monoclonal antibodies were isolated from hybridoma supernatants. Isolated monoclonal antibodies were evaluated, either independently or in combination with other antibodies, for neutralization activity against pseudoviruses containing VP1 proteins of different serotypes.

FIG. 2: Flow cytometry of serum from immunized animals showing binding to VP1-expressing cells. Sera analysis by flow cytometry: EXPI293F cells (Invitrogen) were transfected with BK genotype 1 or 4 VP1, along with VP2 and VP3 in the ratio of 3-1-1. Forty-eight hrs. later, cells were washed and stained with immunized mouse sera (1:200, 1:1000, 1:5000) after washing, and incubated with Allophycocyanin-conjugated goat anti-mouse gamma-chain specific secondary (Jackson Immunoresearch) for 30 mins. Cells were washed again and resuspended in 50 ul PBS to be analyzed on a high throughput flow cytometer (HTFC) (Intellicyt).

FIG. 3: Pseudovirus neutralization by serum from BK immunized Velocimmune mice. 293 TT cells were plated in 96 well plates and 24 hours later incubated with dilutions of mouse serum and pseudovirus-GFP and incubated at RT for 15 mins. Cells were cultured for 72 hrs and PsV infected cells visualized by flow cytometry using GFP as the readout for infection.

FIG. 4: Estimation of EC50 neutralization titers of select supernatants. The concentration of mAb in the supernatants was calculated using the Octet and the amount of protein needed to neutralize against each of the viral genotypes was determined and reported as pM. Infection was determined by expression of GFP in infected cells. The results are expressed as EC50 pM (concentration that inhibits 50% PsV infection). Along the x-axis from left to right, the arrow designates that the order of bars in each cluster of four bars corresponds to order of the legend in the left to right orientation.

FIG. 5: EC50 neutralization using purified monoclonal antibodies. Antibodies from supernatants shown to have neutralizing activity were purified on Protein A columns, resuspended in PBS and tested for neutralization of BK genotype 1, 2, 3 or 4 pseudovirus infection of 293TT cells. The results are expressed as EC50 pM (concentration that inhibits 50% PsV infection). Infection was determined by expression of GFP in infected cells. Along the x-axis from left to right, the arrow designates that the order of bars in each cluster of four bars corresponds to order of the legend in the left to right orientation.

FIG. 6: Neutralization titers of mAb against all four genotypes relative to the comparator antibody. Purified cross neutralizing mAb were tested for neutralization of each genotype PsV in the PsV infection assays using 293TT cells. Infection was determined by expression of GFP in infected cells. Along the x-axis from left to right, the arrow designates that the order of bars in each cluster of four bars corresponds to order of the legend in the left to right orientation.

FIG. 7: Combinations of partial neutralizing antibodies can provide protection against all four genotypes of BK. Mixtures containing pairs of mAbs, and each mixture was tested for neutralization of BK genotype 1, 2, 3 or 4 pseudovirus infection of 293TT cells. The results are expressed as EC50 pM (concentration that inhibits 50% PsV infection). Infection was determined by expression of GFP in infected cells. These results are reproduced in Table 9. Along the x-axis from left to right, the arrow designates that the order of bars in each cluster of four bars corresponds to order of the legend in the left to right orientation.

FIG. 8A: Binding of cross neutralizing mAb to genotype 1 VP1 with select mutations. VP1 with the indicated mutation or unmutated were used to transfect 293TT cells and changes in binding determined by flow cytometer. Colored lines indicate that the mutations affected the binding of the indicated family of antibodies. Families C, H, J, K, and L are shown. The comparator antibody is fully human, and binding is identified using a different secondary antibody. Along the x-axis from left to right, the arrow designates that the order of bars in each cluster of five bars corresponds to order of the legend in the left to right orientation.

FIG. 8B: Location of epitopes on BK virus VP1 targeted by monoclonal antibodies of families C, H, J, K, and L.

FIG. 9: Neutralization and flow cytometric analysis of selected supernatants containing antibodies. Upper portion of table shows neutralization of four pseudoviruses expressing VP1 from each serotype by supernatants containing monoclonal antibodies, and flow cytometry analysis of cells transfected with one of four nucleic acids encoding a different BK virus VP1 serotype, by staining transfected cells with the same supernatants. Neutralization numbers represent percent of infected cells when supernatants were used in neutralization assay. Flow cytometry numbers represent MFI of cells stained using the supernatants transfected with BK I-IV VP I or mock transfected (median). In the lower portion of the table, supernatants are grouped by the similarities of their binding profiles. These results are performed with undiluted culture supernatants with unknown protein concentrations.

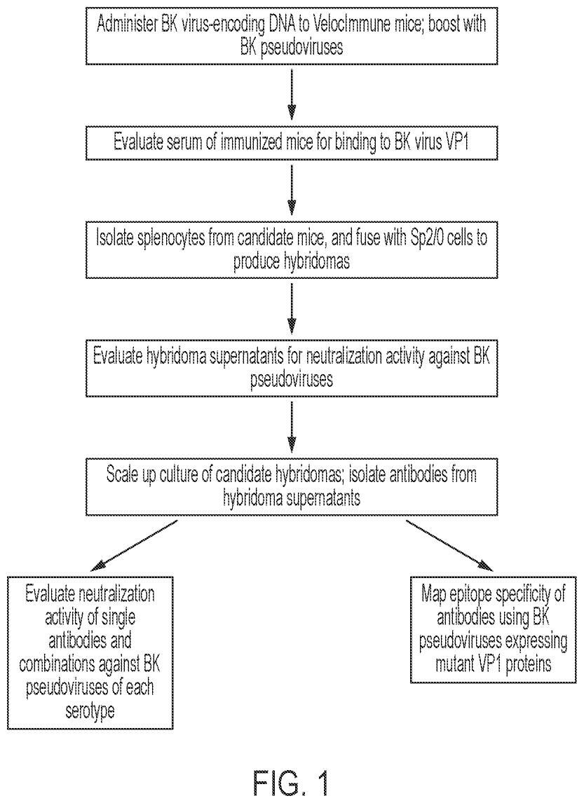

### DETAILED DESCRIPTION OF CERTAIN EMBODIMENTS

### Definitions

As used herein, “antibody” refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies include, but are not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

As used herein, “complementarity-determining domains” or “complementary-determining regions” (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well-known definitions in the art, e.g., Kabat, Chothia, and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HC CDR1), 50-65 (HC CDR2), and 95-102 (HC CDR3) in a VH, e.g., a mammalian VH, e.g., a human VH; and amino acid residues 24-34 (LC CDR1), 50-56 (LC CDR2), and 89-97 (LC CDR3) in a VL, e.g., a mammalian VL, e.g., a human VL.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

As used herein, “antigen binding fragment” refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Nat. Acad. Sci. 85:5879-5883, 1988). Such single chain antibodies are also intended to be encompassed within the term “antigen-binding fragment.” These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8:1057-1062, 1995; and U.S. Pat. No. 5,641,870).

As used herein, “monoclonal antibody” refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

As used herein, a “human antibody” includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J Mol. Biol. 296:57-86, 2000).

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

As used herein, “polypeptide” and “protein” refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

As used herein, “BK virus” and “BKV” refer to a member of the family Polyomaviridae, genus Orthopolyomavirus. Polyomaviruses are icosahedral, non-enveloped, double-stranded DNA viruses with a genome of approximately 5,000 base pairs. They measure approximately 40-45 nM in diameter (Bennett et al., Microbes and Infection. 2012:14(9):672-683).

As used herein, “JC virus” and “JCV” refer to a member of the family Polyomaviridae, genus Orthopolyomavirus. JC virus is related to BK virus, and is also an icosahedral, non-enveloped, double-stranded DNA virus with a genome of approximately 5,000 base pairs. They measure approximately 40-45 nM in diameter (Johne et al., Arch. Virol. 2011; 156(9):1627-1634).

As used herein, “serotype” refers to a subset of a virus defined by the immune response generated by viruses of that subset. When exposed to a virus of a given serotype, a host, such as a mouse or human, generates antibodies that bind to that virus and to other viruses of the same serotype, but not necessarily to other viruses of different serotypes. The serotype of a virus can be determined by contacting the virus with antibodies generated in response to viruses of each known serotype, and determining which antibodies bind to the virus. If antibodies generated in response to serotype 1 bind to the virus in question, the virus is said to be of serotype 1.

Sequence identity can be determined using the methods described herein, for example, aligning two sequences using BLAST, ALIGN, CLUSTAL, CLUSTALW, or another alignment software or algorithm known in the art. Percent (%) sequence identity, with respect to a reference amino acid sequence, refers to the percentage of amino acids in a candidate sequence that are identical to the amino acids in the reference amino acid sequence, after aligning the sequences and introducing gaps, if necessary, to achieve maximum percent sequence identity. The percent sequence identity between a candidate amino acid sequence (A) and amino acid sequence (B) can be calculated as follows: % sequence identity=100 times X/Y, where X is the number of amino acids in A that are identical to the amino acids in the corresponding positions in B, after alignment, and Y is the total number of amino acids in B. In some embodiments, the amino acid sequence comprises at least 90%, at least 91%, at least 92%, at least 93%, at least 94%, at least 95%, at least 96%, at least 97%, at least 98%, or at least 99% sequence identity to a reference sequence.

As used herein, “neutralization” refers to the inhibition of viral infection of a host cell, as demonstrated by the absence of viral gene expression. Without being held to any one theory, mechanisms of neutralization by a particular antibody could include blocking the interaction of viral capsid proteins with cell surface receptors or disruption of any stage of the entry and trafficking process prior to delivery of the viral genome to the nucleus of the host cell.

As used herein “subject” refers to human and non-human animals. Non-human animals include all vertebrates, e.g., mammals and non-mammals, such as non-human primates, sheep, dog, cow, chickens, amphibians, and reptiles.

As used herein, the terms “treat,” “treating,” or “treatment” of any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The phrase “reducing the likelihood” refers to delaying the onset or development or progression of the disease, infection or disorder.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in severity of, respectively, disease symptoms, including symptoms associated polyomavirus infection.

### Antibodies

The present disclosure provides, in some aspects, isolated antibodies and antibody fragments (e.g., antigen-binding fragments) that bind to VP1 of a polyomavirus, such as BK virus. An antibody is a soluble form of a B cell receptor, expressed by B cells, that is capable of binding to an epitope or antigen. An antibody is capable of binding to an epitope if the antibody non-transiently associates with the epitope or antigen in a noncovalent manner, such as through hydrogen bonding. The exact strength of binding will depend on the structure of the antibody and the epitope or antigen in question. Methods of determining whether an antibody binds to an epitope or antigen are well known in the art.

In some embodiments, an antibody or antigen-binding fragment thereof comprises one or more heavy chains and one or more light chains. In some embodiments, an antibody or antigen-binding fragment comprises two heavy chains and two light chains, wherein a first light chain is linked to a first heavy chain by a disulfide bond, a second light chain is linked to a second heavy chain by a disulfide bond, and the first and second heavy chains are linked by a disulfide bond. A heavy chain, in some embodiments, comprises a variable domain (VH), comprising three complementarity determining regions (CDRs), and one or more constant domains (CH). A light chain, in some embodiments, comprises a variable domain comprising CDRs (VL) and one or more constant domains.

In some embodiments, the structure of an antibody, and consequently whether the antibody is capable of binding to an epitope or antigen, is determined in part by the amino acid sequences of the CDRs, VH domain, and/or VL domain. The nucleic acid sequence encoding an antibody, and thus the amino acid sequence of the antibody, is determined during B cell development, such that any two B cells often express antibodies with different amino acid sequences. B cells develop from hematopoietic stem cells in the bone marrow, during which the genome of the cell undergoes recombination that results in the formation of the nucleic acid sequence encoding the VH domain and the nucleic acid sequence encoding the VL domain. Prior to the recombination that forms the nucleic acid encoding the VH domain, the genome of a hematopoietic stem cell comprises multiple heavy chain variable (V) segments, multiple heavy chain diversity (D) segments, and multiple heavy chain joining (J) segments. In a developing B cell, the chromosome is recombined such that the DNA between one D and one J segment is excised from the chromosome, forming a DJ complex comprising the D segment and J segment. Next, the chromosome is recombined such that the DNA between one V segment and the DJ complex is excised from the chromosome, forming a VDJ complex comprising the V segment and the DJ complex. The recombination that forms the nucleic acid encoding the VL domain occurs in a similar manner, except that the nucleic acid encoding the VL domain comprises a V segment and a J segment, but not a D segment, and thus formation of the nucleic acid encoding VL domain requires only one recombination step that forms a VJ complex from a V segment and a J segment. Prior to this recombination, the genome of a hematopoietic stem cell comprises multiple light chain variable (V) segments and multiple joining (J) segments. Following the formation of the heavy chain VDJ complex, the chromosome comprising the light chain V and J segments is recombined to excise DNA between one V segment and J segment, forming a VJ complex.

In some embodiments, the present disclosure provides an antibody or antigen-binding fragment thereof comprising a heavy chain variable region (VH) having three complementarity determining regions (CDRs) of HCDR1, HCDR2, and HCDR3, and a light chain variable region (VL) having three CDRs of LCDR1, LCDR2, and LCDR3. Representative amino acid sequences of the CDRs of antibodies provided herein are shown in Table 1, nucleotide sequences are shown in Table 5. In some embodiments, (a) HCDR1 comprises at least 90% sequence identity to any one of SEQ ID NO: 1, 9, 17, 25, 33, 41, 49, 57, 65, 73, 81, 89, 97, 105, 113, 121, 129, 137, 145, 153, 161, 169, or 177;
(b) HCDR2 comprises at least 90% sequence identity to any one of SEQ ID NO: 2, 10, 18, 26, 34, 42, 50, 58, 66, 74, 82, 90, 98, 106, 114, 122, 130, 138, 146, 154, 162, 170, or 178;
(c) HCDR3 comprises at least 90% sequence identity to any one of SEQ ID NO: 3, 11, 19, 27, 35, 43, 51, 59, 67, 75, 83, 91, 99, 107, 115, 123, 131, 139, 147, 155, 163, 171, or 179;
(d) LCDR1 comprises at least 90% sequence identity to any one of SEQ ID NO: 4, 12, 20, 28, 36, 44, 52, 60, 68, 76, 84, 92, 100, 108, 116, 124, 132, 140, 148, 156, 164, 172, or 180;
(e) LCDR2 comprises at least 90% sequence identity to any one of SEQ ID NO: 5, 13, 21, 29, 37, 45, 53, 61, 69, 77, 85, 93, 101, 109, 117, 125, 133, 141, 149, 157, 165, 173, or 181; and
(f) LCDR3 comprises at least 90% sequence identity to any one of SEQ ID NO: 6, 14, 22, 30, 38, 46, 54, 62, 70, 78, 86, 94, 102, 110, 118, 126, 134, 142, 150, 158, 166, 174, or 182.

In some embodiments, HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 9, HCDR2 comprises SEQ ID NO: 10, HCDR3 comprises SEQ ID NO: 11, LCDR1 comprises SEQ ID NO: 12, LCDR2 comprises SEQ ID NO: 13, and LCDR3 comprises SEQ ID NO: 14, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 17, HCDR2 comprises SEQ ID NO: 18, HCDR3 comprises SEQ ID NO: 19, LCDR1 comprises SEQ ID NO: 20, LCDR2 comprises SEQ ID NO: 21, and LCDR3 comprises SEQ ID NO: 22, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 25, HCDR2 comprises SEQ ID NO: 26, HCDR3 comprises SEQ ID NO: 27, LCDR1 comprises SEQ ID NO: 28, LCDR2 comprises SEQ ID NO: 29, and LCDR3 comprises SEQ ID NO: 30, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 33, HCDR2 comprises SEQ ID NO: 34, HCDR3 comprises SEQ ID NO: 35, LCDR1 comprises SEQ ID NO: 36, LCDR2 comprises SEQ ID NO: 37, and LCDR3 comprises SEQ ID NO: 38, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 41, HCDR2 comprises SEQ ID NO: 42, HCDR3 comprises SEQ ID NO: 43, LCDR1 comprises SEQ ID NO: 44, LCDR2 comprises SEQ ID NO: 45, and LCDR3 comprises SEQ ID NO: 46, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 49, HCDR2 comprises SEQ ID NO: 50, HCDR3 comprises SEQ ID NO: 51, LCDR1 comprises SEQ ID NO: 52, LCDR2 comprises SEQ ID NO: 53, and LCDR3 comprises SEQ ID NO: 54, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 65, HCDR2 comprises SEQ ID NO: 66, HCDR3 comprises SEQ ID NO: 67, LCDR1 comprises SEQ ID NO: 68, LCDR2 comprises SEQ ID NO: 69, and LCDR3 comprises SEQ ID NO: 70, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 73, HCDR2 comprises SEQ ID NO: 74, HCDR3 comprises SEQ ID NO: 75, LCDR1 comprises SEQ ID NO: 76, LCDR2 comprises SEQ ID NO: 77, and LCDR3 comprises SEQ ID NO: 79, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 81, HCDR2 comprises SEQ ID NO: 82, HCDR3 comprises SEQ ID NO: 83, LCDR1 comprises SEQ ID NO: 84, LCDR2 comprises SEQ ID NO: 85, and LCDR3 comprises SEQ ID NO: 86, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 89, HCDR2 comprises SEQ ID NO: 90, HCDR3 comprises SEQ ID NO: 91, LCDR1 comprises SEQ ID NO: 92, LCDR2 comprises SEQ ID NO: 93, and LCDR3 comprises SEQ ID NO: 94, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 97, HCDR2 comprises SEQ ID NO: 98, HCDR3 comprises SEQ ID NO: 99, LCDR1 comprises SEQ ID NO: 100, LCDR2 comprises SEQ ID NO: 101, and LCDR3 comprises SEQ ID NO: 102, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 105, HCDR2 comprises SEQ ID NO: 106, HCDR3 comprises SEQ ID NO: 107, LCDR1 comprises SEQ ID NO: 108, LCDR2 comprises SEQ ID NO: 109, and LCDR3 comprises SEQ ID NO: 110, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 113, HCDR2 comprises SEQ ID NO: 114, HCDR3 comprises SEQ ID NO: 115, LCDR1 comprises SEQ ID NO: 116, LCDR2 comprises SEQ ID NO: 117, and LCDR3 comprises SEQ ID NO: 118, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 121, HCDR2 comprises SEQ ID NO: 122, HCDR3 comprises SEQ ID NO: 123, LCDR1 comprises SEQ ID NO: 124, LCDR2 comprises SEQ ID NO: 125, and LCDR3 comprises SEQ ID NO: 126, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 129, HCDR2 comprises SEQ ID NO: 130, HCDR3 comprises SEQ ID NO: 131, LCDR1 comprises SEQ ID NO: 132, LCDR2 comprises SEQ ID NO: 133, and LCDR3 comprises SEQ ID NO: 134, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 137, HCDR2 comprises SEQ ID NO: 138, HCDR3 comprises SEQ ID NO: 139, LCDR1 comprises SEQ ID NO: 140, LCDR2 comprises SEQ ID NO: 141, and LCDR3 comprises SEQ ID NO: 142, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 145, HCDR2 comprises SEQ ID NO: 146, HCDR3 comprises SEQ ID NO: 147, LCDR1 comprises SEQ ID NO: 148, LCDR2 comprises SEQ ID NO: 149, and LCDR3 comprises SEQ ID NO: 150, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 153, HCDR2 comprises SEQ ID NO: 154, HCDR3 comprises SEQ ID NO: 155, LCDR1 comprises SEQ ID NO: 156, LCDR2 comprises SEQ ID NO: 157, and LCDR3 comprises SEQ ID NO: 158, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 161, HCDR2 comprises SEQ ID NO: 162, HCDR3 comprises SEQ ID NO: 163, LCDR1 comprises SEQ ID NO: 164, LCDR2 comprises SEQ ID NO: 165, and LCDR3 comprises SEQ ID NO: 166, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 169, HCDR2 comprises SEQ ID NO: 170, HCDR3 comprises SEQ ID NO: 171, LCDR1 comprises SEQ ID NO: 172, LCDR2 comprises SEQ ID NO: 173, and LCDR3 comprises SEQ ID NO: 174, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, HCDR1 comprises SEQ ID NO: 177, HCDR2 comprises SEQ ID NO: 178, HCDR3 comprises SEQ ID NO: 179, LCDR1 comprises SEQ ID NO: 180, LCDR2 comprises SEQ ID NO: 181, and LCDR3 comprises SEQ ID NO: 182, or an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

The present disclosure provides, in some aspects, antibodies or antigen-binding fragments thereof, comprising a heavy chain variable region (VH) and a light chain variable region (VL). In some embodiments, VH comprises at least 90% sequence identity to any one of SEQ ID NO: 7, 15, 23, 31, 39, 47, 55, 63, 71, 79, 87, 95, 103, 111, 119, 127, 135, 143, 151, 159, 167, 175, or 183; and VL comprises at least 90% sequence identity to any one of SEQ ID NO: 8, 16, 24, 32, 40, 48, 56, 64, 72, 80, 88, 96, 104, 112, 120, 128, 136, 144, 152, 160, 168, 176, or 184. In some embodiments, VH comprises SEQ ID NO: 7, and VL comprises SEQ ID NO: 8.

In some embodiments, VH comprises SEQ ID NO: 15, and VL comprises SEQ ID NO: 16. In some embodiments, VH comprises SEQ ID NO: 31, and VL comprises SEQ ID NO: 32. In some embodiments, VH comprises SEQ ID NO: 39, and VL comprises SEQ ID NO: 40. In some embodiments, VH comprises SEQ ID NO: 47, and VL comprises SEQ ID NO: 48. In some embodiments, VH comprises SEQ ID NO: 55, and VL comprises SEQ ID NO: 56. In some embodiments, VH comprises SEQ ID NO: 63, and VL comprises SEQ ID NO: 64. In some embodiments, VH comprises SEQ ID NO: 71, and VL comprises SEQ ID NO: 72. In some embodiments, VH comprises SEQ ID NO: 79, and VL comprises SEQ ID NO: 80. In some embodiments, VH comprises SEQ ID NO: 87, and VL comprises SEQ ID NO: 88. In some embodiments, VH comprises SEQ ID NO: 95, and VL comprises SEQ ID NO: 96. In some embodiments, VH comprises SEQ ID NO: 103, and VL comprises SEQ ID NO: 104. In some embodiments, VH comprises SEQ ID NO: 111, and VL comprises SEQ ID NO: 112. In some embodiments, VH comprises SEQ ID NO: 119, and VL comprises SEQ ID NO: 120. In some embodiments, VH comprises SEQ ID NO: 127, and VL comprises SEQ ID NO: 128. In some embodiments, VH comprises SEQ ID NO: 135, and VL comprises SEQ ID NO: 136. In some embodiments, VH comprises SEQ ID NO: 143, and VL comprises SEQ ID NO: 144. In some embodiments, VH comprises SEQ ID NO: 151, and VL comprises SEQ ID NO: 152. In some embodiments, VH comprises SEQ ID NO: 159, and VL comprises SEQ ID NO: 160. In some embodiments, VH comprises SEQ ID NO: 167, and VL comprises SEQ ID NO: 168. In some embodiments, VH comprises SEQ ID NO: 175, and VL comprises SEQ ID NO: 176. In some embodiments, VH comprises SEQ ID NO: 183, and VL comprises SEQ ID NO: 184. In other embodiments, the VH or VL may have an amino acid sequence having at least 80%, at least 85%, at least 90%, at least 91%, at least 92%, at least 93%, at least 94%, at least 95%, at least 96%, at least 97%, at least 98%, or at least 99% sequence identity to any of the aforementioned sequences.

In some embodiments, an antibody or antigen-binding fragment thereof comprises heavy chain framework regions and/or light chain framework regions. Framework regions are amino acid sequences present in VH or VL domains that are distinct from the CDRs and support the formation of the VH or VL domains. In some embodiments, the antibody or antigen-binding fragment comprises heavy chain framework regions HFR1, HFR2, HFR3, and HFR4, and light chain framework regions LFR1, LFR2, LFR3, and LFR4. Representative amino acid sequences of framework regions of antibodies provided herein are shown in Table 2. In some embodiments, the HFR1 region comprises the amino acid sequence of any one of SEQ ID NOs: 185, 193, 197, 205, 213, 226, 234, 241, 250, or 269, the HFR2 region comprises the amino acid sequence of any one of SEQ ID NOs: 186, 194, 198, 206, 214, 223, 227, 232, 242, or 253, the HFR3 region comprises the amino acid sequence of any one of SEQ ID NOs: 187, 195, 199, 207, 215, 219, 224, 228, 233, 235, 243, 251, 254, 258, 259, 262, 265, 267, or 270, the HFR4 region comprises the amino acid sequence of any one of SEQ ID NOs: 188, 200, 208, 220, 229, 236, 244, or 271, the LFR1 region comprises the amino acid sequence of any one of SEQ ID NOs: 189, 201, 209, 237, 245, 255, or 260, the LFR2 region comprises the amino acid sequence of any one of SEQ ID NOs: 190, 202, 210, 216, 221, 225, 230, 238, 246, 261, 263, or 272, the LFR3 region comprises SEQ ID NOs: 191, 196, 203, 211, 217, 222, 230, 239, 247, 249, 252, 256, 264, 266, 268, or 273, and the LFR4 region comprises the amino acid sequence of any one of SEQ ID NOs: 192, 204, 212, 218, 240, 248, or 257.

In some embodiments, an antibody or antigen-binding fragment thereof comprises one or more heavy CH domains and/or one or more CL domains. CH and CL domains are members of the immunoglobulin superfamily, each comprising multiple β-strands. In some embodiments, an antibody or antigen-binding fragment thereof comprises three, four, five, six, seven, or eight CH domains. In some embodiments, one or more CH domains are selected from the group consisting of IgG, IgG1, IgG2, IgG2a, IgG2b, IgG2c, IgG3, IgG4, IgA, IgA1, IgA2, IgD, IgM, and IgE constant domains. Representative amino acid sequences of the heavy chain CH domains are provided in Table 3. Two antibodies may have identical VH and VL domains, and therefore bind to the same epitope or antigen, but comprise different CH domains, and thus differ in structure. When a B cell first becomes activated after the B cell receptor or antibody on its surface binds to an epitope or antigen, expression of a soluble antibody begins with the step of transcribing a pre-mRNA, in which nucleic acid sequence encoding the IgM CH domain and a nucleic acid sequence encoding the IgD CH domain are located downstream from the nucleic acid sequence encoding the VH domain. During pre-mRNA processing, the pre-mRNA is spliced such that the nucleic acid sequence encoding the IgM CH domain, or the nucleic acid sequence encoding the IgD CH domain, is adjacent to the nucleic acid sequence encoding the VH domain, such that translation of the mRNA produces an IgM or IgD heavy chain. The isotype of an antibody refers to the CH domain of the antibody (e.g., an antibody with an IgG CH domain has an IgG isotype). A B cell genome comprising a nucleic acid sequence encoding an antibody may undergo recombination, that results in the excision of a nucleic acid sequence encoding one or more CH domains, such that a nucleic acid sequence encoding a different CH domain becomes the first CH domain-encoding nucleic acid that downstream of the VDJ-encoding nucleic acid. The antibody produced by a B cell with a genome that has been recombined in this manner is then of a different isotype than IgM or IgD, such as IgG, IgG1, IgG2, IgG2a, IgG2b, IgG2c, IgG3, IgG4, IgA, IgA1, IgA2, or IgE.

In some embodiments, one or more CL domains are selected from the group consisting of Ig kappa and Ig lambda constant domains. Representative amino acid sequences of the heavy chain CH domains are provided in Table 4. In some animals, including mice and humans, the genome comprises two loci, referred to as the Ig kappa locus and the Ig lambda locus, that are capable of recombination to form a nucleic acid sequence encoding a VL domain. In developing B cells, recombination occurs at the Ig kappa locus or the Ig lambda locus, to place a light chain V segment adjacent to a light chain J segment, and if recombination is successful in generating a VL domain, then the light chain that is expressed comprises the Ig kappa or Ig lambda CL domain, respectively.

In some embodiments, one or more CH domains comprise an amino acid sequence with at least 90% sequence identity to one of SEQ ID NOs: 274-290. In some embodiments, an antibody or antigen-binding fragment thereof comprises one or two CL domains. In some embodiments, one or more CL domains comprise an amino acid sequence with at least 90% sequence identity to one of SEQ ID NOs: 291-301.

The present disclosure provides, in some aspects, an isolated antibody or antigen-binding fragment thereof, wherein, when bound to BK virus VP1 protein, the antibody binds to at least one of the following residues: S80, D82, R83, or R170 of SEQ ID NO: 529, and wherein the antibody blocks binding of BK virus VP1 to sialic acid. An example sequence of BK virus VP1 protein is given by Accession No. P03088, which is reproduced as SEQ ID NO: 529. Whether an antibody binds to a given residue of a protein may be determined by many methods of epitope mapping known in the art, such as X-ray co-crystallography or mutagenesis mapping (see, e.g., King et al. Methods Mol Biol. 2018. 1785:13-27 and Davidson et al. Immunology. 2014. 143(1):13-20). X-ray co-crystallography is the process of crystallizing an antibody that is bound to an antigen and using X-ray analysis to visualize the interaction between the antibody and antigen. The location at which an antibody binds to the antigen can then be observed from this visualization. Mutagenesis mapping refers to the process of generating modified forms of a protein, such as VP1, and determining how well an antibody binds to the modified forms of the protein relative to the unmodified protein. For example, if an antibody binds to a protein with the amino acid sequence set forth in SEQ ID NO: 529, but does not bind to a protein with the same amino acid sequence in which the seine at position 80 (S80) is substituted for an alanine, then the antibody is said to bind to residue S80.

The present disclosure provides, in some aspects, an isolated antibody or antigen-binding fragment thereof, wherein (a) the antibody is capable of inhibiting infection by a virus comprising VP1 of BK virus serotype 1, a virus comprising VP1 of BK virus serotype 2, a virus comprising VP1 of BK virus serotype 3, and a virus comprising VP1 of BK virus serotype 4, wherein an antibody capable of inhibiting infection by a virus is an antibody that inhibits infection of a target cell by a virus, as determined by a method comprising the steps of:
(i) contacting the virus with the antibody or a control protein;
(ii) incubating the virus with target cells;
(iii) determining the proportion of the target cells infected by the virus, or the amount of viral RNA or DNA present in the target cells, at the end of step (ii);
   wherein determination of a lesser proportion of target cells infected by the virus, or a lesser amount of pseudoviral RNA or DNA present in the target cells, in the presence of the antibody, relative to the amount of target cells infected or amount of viral RNA or DNA in the absence of the antibody, is indicative of inhibition of infection by the virus; and
   (b) the antibody is capable of binding to an epitope that is present on VP1 of BK virus serotype 1, VP1 of BK virus serotype 2, VP1 of BK virus serotype 3, and VP1 of BK virus serotype 4.

Infection of a cell by BK virus begins with binding of BK virus VP1 to a sialic residue (e.g., α2,8-linked sialic acid) on the surface of the cell. Following sialic binding, endocytosis forms an endosome containing the virus particle, after which the VP1-containing capsid uncoats to facilitate viral entry into the cytoplasm. Binding of an antibody to an epitope on the viral VP1 protein can interfere with the function of VP1. For example, if amino acids of the epitope to which the antibody is bound are required for adherence to sialic acid, then steric hindrance by the bound antibody can prevent antibody-bound VP1 from binding to the host cell surface. Alternatively, antibody binding may inhibit the uncoating process, such that endocytosed virus particles are unable to enter the cytoplasm.

Determining whether an antibody inhibits infection by a virus comprising a given VP1 protein, or a VP1 protein of a given serotype, may be achieved by one of multiple methods known in the art, such as a virus or pseudovirus neutralization assay. A pseudovirus is a virus that is capable of infecting a cell, but not replicating or producing new virus particles in that cell, often because it lacks at least one nucleic acid required for the production of new virus particles.

Pseduoviruses may comprise the same viral proteins as a virus of interest, and optionally a reporter protein, such as green fluorescent protein, which allows one of ordinary skill in the art to measure the ability of the pseudovirus to infect target cells. For example, the pseudovirus may be incubated with a population of target cells for a defined period of time. Following that period of time, the number or proportion of cells infected may be measured by one of multiple methods known in the art. For example, if the pseudovirus comprises a reporter protein, such as green fluorescent protein, the number of cells in which GFP is present may be measured by flow cytometry, and used to calculate the proportion of cells infected. A similar approach may be used to measure the infectivity of the virus of interest, provided the virus comprises a reporter protein. Alternatively, after the virus is incubated with target cells and residual, non-infecting virus particles are removed from the surfaces of target cells, the amount of viral DNA or viral RNA present in the target cells may be quantified by PCR or RT-qPCR.

Determining whether an antibody of interest or antigen-binding fragment inhibits infection by a virus comprising a given protein, such as a VP1 protein of a given serotype, may be accomplished by similar approaches to those described in the preceding paragraph, with the addition of a first step, in which the virus or pseudovirus is contacted with the antibody or a control protein before the step of incubating the virus or pseudovirus with target cells. A control protein refers to a protein that is not known to bind to the virus or pseudovirus. Non-limiting examples of control proteins include bovine serum albumin and antibodies of the same isotype as the antibody of interest that binds to an epitope or antigen that is not present on the virus or pseudovirus.

To determine whether the antibody inhibits infection, the proportion of target cells infected when the virus or pseudovirus was contacted with the antibody is compared to the proportion of target cells infected when the virus or pseudovirus was contacted with the control protein. Alternatively, the total amount of viral RNA or DNA present in the target cells following incubation with the virus or pseudovirus may be measured in both cases. If the proportion of cells infected, or total amount of viral RNA or DNA, is lower when the virus or pseudovirus is contacted with the antibody, compared to the control protein, then the antibody is said to inhibit infection by the virus or pseudovirus.

The present disclosure provides, in some aspects, an isolated antibody or antigen-binding fragment thereof that competes for binding to BK virus VP1 protein with an antibody produced by a hybridoma. Methods for generating hybridomas, and generating monoclonal antibodies using hybridoma technology, are known in the art (Antibody Methods and Protocols, Methods in Molecular Biology vol. 901, 2012, Chapter 7: 117). Briefly, B cells from an animal, such as a mouse or human, are mixed with immortalized myeloma cells, and exposed to electricity so that a B cell may fuse with a myeloma cell, producing a hybridoma, an immortal cell capable of producing the antibody encoded by the B cell. Individual hybridoma clones may be isolated, proliferated, and preserved. Culturing a clonal lineage derived from a single hybridoma cell results in the production of a monoclonal antibody, where each antibody in the culture comprises a substantially identical amino acid sequence.

In some embodiments, the antibody or antigen-binding fragment thereof has a dissociation constant (K_{D}) from BK virus VP1 protein selected from the group consisting of at most about 10⁻⁷ M; at most about 10⁻⁸ M; at most about 10⁻⁹ M; at most about 10⁻¹⁰ M; at most about 10⁻¹¹ M; at most about 10⁻¹² M; and at most 10⁻¹³ M. The dissociation constant K_{D} of an antibody from an epitope or antigen refers to the rate is a measure of the affinity of the antibody for the epitope or antigen, with a lower K_{D} representing a higher affinity for the epitope or antigen. In reference to an antibody and a given epitope or antigen, K_{D} is defined as the concentration of an antibody at which half of the antibody molecules in a solution are associated with the epitope or antigen. Methods of measuring the K_{D} of an antibody with respect to an epitope or antigen are well known in the art (see, e.g., Pichler et al. J Immunol. Methods. 1997; 201(2):189-206).

In some embodiments, the antibody is a monoclonal antibody or antigen-binding fragment thereof. The term “monoclonal antibody” refers to proteins or polypeptides derived from the same genetic source, and thus having substantially identical amino acid sequences.

In some embodiments, the antibody is a chimeric antibody or antigen-binding fragment thereof. A chimeric antibody is an antibody comprising amino acid sequences from different genetic sources. In some embodiments, the chimeric antibody comprises amino acid sequences from a mouse and amino acid sequences from a human. In some embodiments a chimeric antibody comprises a variable domain derived from a mouse and constant domains derived from a human.

In some embodiments, the antibody is a humanized antibody or antigen-binding fragment thereof. A humanized antibody is an antibody comprising constant domains and framework regions derived from a human. In some embodiments, a humanized antibody comprises one or more CDRs derived from a non-human animal. Non-limiting examples of non-human animals from which CDRs may be derived include mice, rats, hamsters, rabbits, goats, sheep, and non-human primates.

In some embodiments, the antibody is a human engineered antibody. A human engineered antibody refers to an antibody derived from a non-human source, such as mouse, in which one or more substitutions have been made to improve a desired characteristic of the antibody, such as to increase stability or reduce immunogenicity when the antibody is administered to a subject. In some embodiments, the substitutions are made at low-risk positions (e.g., exposed to solvent but not contributing to antigen binding or antibody structure). Such substitutions mitigate the risk that a subject will generate an immune response against the antibody following its administration, without affecting the ability of the antibody to bind to a desired epitope or antigen (see, e.g., Studnicka et al. Protein Eng. 1994. 7(6):805-814).

In some embodiments, the antibody is a single chain antibody or antigen-binding fragment. A single chain antibody, or single chain variable fragment (scFV) is a protein or polypeptide comprising a VH domain and a VL domain joined together, such as by a synthetic linker, to form a single protein or polypeptide (see, e.g., Bird et al., Science. 242:423-426, 1988; and Huston et al., Proc. Natl. Acad. Sci. 85:5879-5883, 1988).

In some embodiments, the antibody is an antibody fragment or antigen-binding fragment. An antibody fragment is protein or polypeptide derived from an antibody. An antigen-binding fragment is a protein or polypeptide derived from an antibody that is capable of binding to the same epitope or antigen as the antibody from which it was derived.

In some embodiments, the antibody has reduced glycosylation, no glycosylation, or is hypofucosylated. Glycosylation refers to the covalent attachment of sugar, monosaccharide, disaccharide, oligosaccharide, polysaccharide, or glycan moieties to a molecule, such as a polypeptide or protein. These sugar or glycan moieties are generally attached to an antibody in a post-translational matter, prior to secretion by a B cell. An antibody with reduced glycosylation has fewer of these attached sugar or glycan moieties than the number that are typically attached to an antibody with a substantially identical amino acid sequence, such as when the antibody is produced by a B cell in vitro or in vivo in a mouse or human. An antibody with no glycosylation has no attached sugar or glycan moieties. An antibody that is hypofucosylated has fewer fucosyl residues than the number that are typically attached to an antibody with a substantially identical amino acid sequence, such as when the antibody is produced by a B cell in vitro or in vivo in a mouse or human.

### Compositions Comprising Antibodies and Uses Thereof

The present disclosure provides, in some aspects, compositions comprising two or more of the antibodies and/or antigen-binding fragments described herein. In some embodiments, a second antibody comprises at least one CDR sequence which is distinct from at least one CDR sequence of a first CDR sequence. The structure of an antibody, and whether the antibody is capable of binding to a given epitope or antigen is determined in part by the combination of six CDRs (HCDR1, HCDR2, HCDR3, LCDR1, LCDR2, and LCDR3). Thus, two antibodies that differ by at least one CDR are structurally distinct, and may be capable of binding to different epitopes or antigens.

In some embodiments, a second antibody comprises at least one VH sequence which is distinct from at least one VH sequence of a first antibody. In some embodiments, a second antibody comprises at least one VL sequence which is distinct from at least one VL sequence of a first antibody. The structure of an antibody, and whether the antibody is capable of binding to a given epitope or antigen, is determined by the combination of the VH and VL amino acid sequences. Thus, two antibodies that differ by a VH sequence, a VL sequence, or both are structurally distinct, and may be capable of binding to different epitopes or antigens.

The antibodies and/or antigen-binding fragments provided herein may be formulated in a pharmaceutical composition comprising an antibody or antigen-binding fragment, and a pharmaceutically acceptable excipient. A pharmaceutically acceptable excipient can also be incorporated in a formulation and can be any excipient (e.g., carrier) known in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such excipients. In some embodiments, the pharmaceutically acceptable excipient contains histidine or a sugar. In some embodiments, the pharmaceutically acceptable excipient contains sugar. In some embodiments, the pharmaceutically acceptable excipient contains sucrose.

Pharmaceutically acceptable excipients are known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, and silicylate. Each pharmaceutically acceptable excipients used in a pharmaceutical composition of the invention must be “acceptable” in the sense of being compatible with the other ingredients of the formulation and not injurious to the subject. Excipients suitable for a selected dosage form and intended route of administration are well known in the art, and acceptable diluents or carriers for a chosen dosage form and method of administration can be determined using ordinary skill in the art.

In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA (e.g., for transplantation into a subject), hyaluronidase, nanoparticle mimics and combinations thereof.

Routes of administration of the antibodies or antigen-binding fragments provided herein include, for example, intravenous, intramuscular, intraperitoneal, subcutaneous, or intranasal. Thus, in some embodiments, a composition comprising an antibody or antigen-binding fragment may be formulated for intravenous, intramuscular, intraperitoneal, subcutaneous, or intranasal delivery.

In some embodiments of the pharmaceutical compositions provided herein, at least 0.05%, at least 0.1%, at least 0.1%, at least 0.5%, at least 1%, at least 2%, at least 3%, at least 4%, or at least 5% of the antibodies in the composition have an α2,3-linked sialic acid residue. Antibodies may be glycosylated, in which sugars or glyans, such as sialic acid, may be covalently attached to amino acid residues of the antibody. Glycosylation of antibodies, including attachment of sialic acid residues such as by α2,3-linkage, may affect the function of the antibodies (see, e.g., Thomann et al. Bioanalysis. 2019. 11(15):1437-1449).

In some embodiments of the pharmaceutical compositions provided herein, none of the antibodies comprise a bisecting N-acetylglucosamine (GlcNAc). A bisecting N-acetylglucosamine is N-glycosylation in which a β1,4-linked GlcNAc is attached to a core 0-mannose residue. Glycosylation of proteins with a bisecting GlcNAc has been shown to affect numerous biological processes, including cell adhesion and tumor development, and the presence of a bisecting GlcNAc may affect the ability of an antibody to bind to an epitope or antigen or neutralize a virus (see, e.g., Thomann et al. Bioanalysis. 2019. 11(15):1437-1449).

In some embodiments of the pharmaceutical compositions provided herein, the composition is prepared as a lyophilisate. Lyophilization, or freeze-drying, is the process of removing water from a composition by freezing the composition, then drying the frozen composition at low temperature and pressure to allow ice to sublimate away from the frozen composition. The dried form of the composition that remains after sublimation is the lyophilisate.

### Vectors and Cells Comprising Nucleic Acids Encoding Antibodies

The present disclosure provides, in some aspects, nucleic acids encoding an antibody or antigen-binding fragment provided herein. In some embodiments, the nucleic acid comprises a sequence encoding a CDR, VH domain, VL domain, CH domain, CL domain, heavy chain, or light chain provided herein. In some embodiments, the nucleic acid comprises a sequence with at least 90% sequence identity to any one of the CDRs, VH domains, VL domains, CH domains, CL domains, heavy chains, or light chains provided herein. In some embodiments, the nucleic acid sequence is codon-optimized for expression in a cell. A codon-optimized nucleic acid is one that has been modified to encode the same amino acid sequence as an unmodified form of the nucleic acid, but using codons that are preferred in a host cell or organism that is used to produce a protein with the amino acid sequence. The host cell or organism may be, for example, a prokaryotic or eukaryotic cell, such as a yeast cell, Chinese hamster ovary (CHO) cell, a human embryonic kidney 293 (HEK-293) cell, or E. coli cell.

In some aspects, the disclosure provides a vector comprising a nucleic acid encoding an antibody provided herein or an antigen-binding fragment thereof. In some embodiments, the vector comprises a nucleic acid sequence encoding a CDR, VH domain, VL domain, CH domain, CL domain, heavy chain, or light chain provided herein. In some embodiments, the vector comprises a nucleic acid sequence with at least 90% sequence identity to any one of the CDRs, VH domains, VL domains, CH domains, CL domains, heavy chains, or light chains provided herein. Various vectors can be used to express nucleic acids encoding the antibodies or fragments thereof provided herein. A vector may be a DNA or RNA vector, such as a plasmid, bacterial artificial chromosome, human artificial chromosome, or mRNA, comprising the nucleic acid sequence encoding an antibody or fragment thereof. In some embodiments, the vector is a viral vector. Non-limiting examples of viral vectors include retroviral vectors, lentiviral vectors, adeno-associated viral vectors, adenoviral vectors, baculoviral vectors, foamy virus vectors, SV40 viral vectors, MPSV vectors, Epstein-Barr viral vectors, and bacteriophage vectors. Methods of cloning nucleic acid sequences encoding antibodies or fragments thereof into expression vectors are well known in the art, as are methods of producing antibodies or fragments thereof using said vectors (see, e.g., Spidel et al. J Immunol Methods. 2016. 439:50-58).

Vectors for the expression of antibodies or fragments thereof provided herein may also comprise one or more regulatory elements, such as a promoter operably linked to the nucleic acid encoding the antibody or fragment. The promoter may be a constitutive promoter, which continuously drives expression of the nucleic acid sequence to which it is linked. The promoter may be an inducible promoter, which regulates expression of the operably linked nucleic acid sequence such that the sequence is expressed only under certain conditions, such as the presence, absence, or concentration of a specific nucleotide, protein, peptide, or other molecule in the cell.

In some aspects, the disclosure provides an isolated host cell comprising a vector encoding an antibody provided herein or antigen-binding fragment thereof. The host cell may be a prokaryotic cell, such as E. coli. The host cell may be a eukaryotic cell, such as a yeast (e.g., Saccharomyces cerevisiae) cell, Chinese hamster ovary (CHO) cell, human embryonic kidney 293 (e.g., HEK-293, HEK-293A, HEK-293T) cell, hybridoma cell, or insect cell. The choice of host cell will depend on multiple factors, including the particular vector to be used, desired characteristics of the antibody, and aspects of the protein expression process. For example, human cells are more permissive to certain viral vectors, such as lentiviral and adeno-associated viral vectors, while insect cells are more permissive to baculoviral vectors. Additionally, fewer post-translational modifications occur in proteins expressed by prokaryotic cells, in contrast to eukaryotic cells, so expression of the same nucleic acid sequence in from an E. coli cell may result in the production of an antibody with less glycosylation than the same antibody expressed from a human or yeast cell.

In one aspect, the present disclosure provides a process of producing an antibody or antigen-binding fragment thereof provided herein, using a host cell comprising a vector or nucleic acid sequence encoding the antibody or antigen-binding fragment. Methods of producing an antibody using a host cell are well known in the art (see, e.g., Frenzel et al. Front Immunol. 2013. 4:217). Briefly, the host cell is cultivated in a medium that allows for a.) suitable for survival and/or proliferation of the host cell, and b.) expression of the nucleic acid sequence encoding the antibody or antigen-binding fragment. For example, if the nucleic acid sequence is operably linked to an inducible promoter, a reagent may be added to the medium to induce the promoter and drive expression of the nucleic acid sequence encoding the antibody or antigen-binding fragment. If nucleic acid or vector comprising the sequence encoding the antibody or antigen-binding fragment also comprises a selectable marker, such as a sequence encoding resistance to an antibiotic or a selective agent, adding the antibiotic or selective agent promotes the survival of cells that are capable of producing the antibody or antigen-binding fragment. As host cells are cultivated, they express the antibody or antigen-binding fragment, which is secreted into the extracellular medium. Following a desired period of cultivation, the medium may be separated from the cells, and the desired protein may be purified from the medium using methods known in the art, such as column-based purification.

### Therapeutic and Diagnostic Uses

The present disclosure provides, in some aspects, methods of neutralizing a BK virus infection by administering an antibody or antigen-binding fragment provided herein, and optionally a therapeutic agent, to a subject in need thereof. An effective amount, which may also be referred to as a therapeutically effective amount, refers to the amount (e.g., dose) at which a desired clinical result (e.g., reduction in viral load) is achieved in a subject. An effective amount is based, at least in part, on the target tissue, target cell type, means of administration, physical characteristics of the inhibitor, other components of the composition, and other determinants, such as age, body weight, height, sex and general health of the subject. A subject may be a mammal, such as a human, a non-human primate (e.g., Rhesus monkey, chimpanzee), or a rodent (e.g., a mouse or a rat). In some embodiments, the subject is a human subject.

In some embodiments, the subject is diagnosed with a BK virus infection or BK viremia. BK virus infection refers to the presence of BK virus particles in one or more organs or anatomical locations of the subject, such as the kidney, liver, or blood. BK viremia refers to the presence of BK virus particles in the blood of the subject.

In some aspects, the present disclosure provides a method of reducing the risk of transplant rejection in a transplant recipient receiving a donor organ, comprising (i) determining serotype of BK virus present in the donor organ, (ii) selecting an antibody, antigen-binding fragment thereof, or composition comprising an antibody or antigen-binding fragment thereof that neutralizes the serotype of BK virus present in the donor organ, and (iii) administering the selected antibody to the transplant recipient via injection or infusion. Methods of determining are the serotype of a virus are known in the art (Pastrana et al. Plos Pathog. 2012. 8(4):e1002650). Multiple preparations of the virus are prepared, each preparation is contacted with an antibody that binds to viruses of a specific serotype, and it is determined whether each antibody binds to the virus. If an antibody that binds to viruses of a serotype and to the virus in question, the virus is said to belong to the serotype. To determine the serotype of BK virus present in a donor organ, a portion of the donor organ is sampled to isolate BK virus, which may then be prepared for serotyping immediately or replicated in vitro before determining the serotype of the virus present in the donor organ.

In some embodiments, the method further comprises administering a therapeutic agent to the subject. In some embodiments, the therapeutic agent is an immunosuppressive agent. In some embodiments, the immunosuppressive agent is a monophosphate dehydrogenase inhibitor, a purine synthesis inhibitor, a calcineurin inhibitor, or an mTOR inhibitor. In some embodiments, the immunosuppressive agent is mycophenolate mofetil (MMF), mycophenolate sodium, azathioprine, tacrolimus, sirolimus, or cyclosporine. In some embodiments, the therapeutic agent is an additional anti-VP1 antibody.

In some aspects, the present disclosure provides a method of treating or reducing the likelihood of a BK virus or JC virus associated disorder, comprising administering one or more of the antibodies provided herein to a subject. In some embodiments, the disorder is transplant rejection, graft-versus-host disease (GvHD), nephropathy, BKVAN, hemorrhagic cystitis (HC), Progressive Multifocal Leukoencephalopathy (PML), granule cell neuronopathy (GCN), interstitial kidney disease, ureteral stenosis, vasculitis, colitis, retinitis, meningitis, or immune reconstitution inflammatory syndrome (IRIS). In some embodiments, the method further comprises administering a therapeutic agent. In some embodiments, the therapeutic agent is an immunosuppressive agent. In some embodiments, the immunosuppressive agent is a monophosphate dehydrogenase inhibitor, a purine synthesis inhibitor, a calcineurin inhibitor, or an mTOR inhibitor. In some embodiments, the immunosuppressive agent is mycophenolate mofetil (MMF), mycophenolate sodium, azathioprine, tacrolimus, sirolimus, or cyclosporine. In some embodiments, the therapeutic agent is an additional anti-VP1 antibody.

In some embodiments of the methods provided herein, the antibody is administered via injection or infusion. Administration by injection is a process in which an antibody or antigen-binding fragment is delivered to a subject through an apparatus. Injection may deliver a composition to a muscle (intramuscular), into the peritoneal cavity (intraperitoneal), or under the skin (subcutaneous) Administration by infusion is a process in which an antibody or antigen-binding fragment is delivered to a subject in a controlled manner over a period of time, such as by a needle or catheter. Infusion may deliver a composition directly to the bloodstream (intravenous) or under the skin (subcutaneous).

In some embodiments, the antibody or antigen-binding fragment is reconstituted prior to injection. Reconstitution involves preparing a liquid suitable for administration by injection or infusion by adding a liquid, gel, or other excipient to a powder formulation of the antibody.

In some aspects, the present disclosure provides diagnostic reagents comprising an antibody or antigen-binding fragment thereof, which is labeled. A labeled antibody is an antibody to which a label, such as an atom, ion, or molecule is attached by a covalent or ionic bond. In some embodiments, the label is a radiolabel. A radiolabel is an atom, ion, or molecule that is radioactive, such that its presence may be detected by exposure to a radiation-sensitive material (e.g., radiographic film). In some embodiments, the label is a fluorophore. A fluorophore is a molecule that is capable of being excited by light, and, following excitation by light of a first wavelength, is capable of emitting light of a second wavelength. A chromophore is also a molecule that emits light of a second wavelength after excitation by light of a first wavelength, wherein the light emitted is in the visible spectrum. In some embodiments, the label is an imaging agent. An imaging agent, also referred to as a contrast agent, that emits energy in the form of X-rays, gamma rays, radio waves, or radioactive particles, such that its presence and location in a subject in a subject can be detected. In some embodiments, the label is a metal ion.

TABLE 1
Amino acid sequences of antibody complementary determining regions,
variable heavy chain domains, and variable light chain domains.

| | Complementarity | Variable Heavy | Variable Light |
|---|---|---|---|
| Antibody | Determining Region | Chain (VH) | Chain (VL) |
| Clone | (CDR) sequences | Sequence | Sequence |
| 1E2 | GFTFSDYY - SEQ ID NO: 1 | QVQLVESGGDLVKPG | DIVMTQSPDSLAVSL |
| | IDSSAGAI - SEQ ID NO: 2 | GSLRLSCVASGFTFS | GERATINCKSSQNIL |
| | ARERRDGHKIFDC - SEQ ID | DYYMNWIRLPPGKGL | YSSNNKNYLAWYQQK |
| | NO: 3 | EWVSYIDSSAGAIYY | AGQPPKLLIFWSSTR |
| | QNILYSSNNKNY - SEQ ID | ADSVRGRFTVSRDDA | ESGVPDRFSGSGSGT |
| | NO: 4 | KNSLFLHMNSLRADD | DFTLTINSLQAEDVA |
| | WSS - SEQ ID NO: 5 | TAVYYCARERRDGHK | VYYCQQYYSSPYNFG |
| | QQYYSSPYN - SEQ ID NO: | IFDCWGPGTLVTVSS - | RGTTLEIK - SEQ |
| | 6 | SEQ ID NO: 7 | ID NO: 8 |
| 1H10 | GFTFSDYY - SEQ ID NO: 9 | QVHLVESGGDLVKPG | DIVMTQSPDSLAVSL |
| | IDSSAGAI - SEQ ID NO: | GSLRLSCVASGFTFS | GERATINCKSSQNIL |
| | 10 | DYYMNWIRMPPGKGL | YSSNNKNYLAWYQQK |
| | ARERRDGHKIFDC - SEQ ID | EWISYIDSSAGAIYY | AGQPPKLLIFWSSTR |
| | NO: 11 | ADSVKGRFTVSRDDA | ESGVPDRFSGSGSGT |
| | QNILYSSNNKNY - SEQ ID | KNSLFLHMNNLRADD | DFTLTISSLQAEDVA |
| | NO: 12 | TAVYYCARERRDGHK | VYYCQQYYSSPYNFG |
| | WSS - SEQ ID NO: 13 | IFDCWGPGTLVTVSS - | RGTTLEIK - SEQ |
| | QQYYSSPYN - SEQ ID NO: 14 | SEQ ID NO: 15 | ID NO: 16 |
| 2A10 | GYSFTYYW - SEQ ID NO: | EVQLVQSGAEVKKPG | DIQMTQSPSSLSASV |
| | 17 | ESLKISCKGSGYSFT | GDRLTITCRASQNIN |
| | IYPGDSDT - SEQ ID NO: 18 | YYWIGWVRQMPGKGL | TFLNWYQQNPGKAPK |
| | TTHARNWNNVAY - SEQ ID | EWMGIIYPGDSDTRY | VLIYAASSLESGVPS |
| | NO: 19 | SPSFQGQVTISADKS | RFSGSGSGTDFTLTI |
| | QNINTE - SEQ ID NO: 20 | ISTAYLQWSSLKASD | SSLQPEDFATYYCQQ |
| | AAS - SEQ ID NO: 21 | TAMYYCTTHARNWNN | SSSTPWTFGQGTKVE |
| | QQSSSTPWT - SEQ ID NO: | VAYWGQGTLVTVSS - | IK - SEQ ID NO: |
| | 22 | SEQ ID NO: 23 | 24 |
| 2B9 | GFTFSGFA - SEQ ID NO: | EVQLLESGGGLAQPG | DIQMTQSPSSLSASV |
| | 25 | KSLRLSCAASGFTFS | GDRVTITCRASQSIS |
| | ISGSGDIT - SEQ ID NO: | GFAMSWVRQAPGKGL | SYLNWYQQKPGKAPK |
| | 26 | EWVSGISGSGDITYY | |
| | AKDPYNWNHGVYGMDV - SEQ | ADSVKGRFTISRDNS | FLIYGASSLQSGVPS |
| | ID NO: 27 | KSTLYLQMNSLGAED | RFSGSGSGTDETLTI |
| | QSISSY - SEQ ID NO: 28 | TAVYYCAKDPYNWNH | SSLQPEDFAAYYCQQ |
| | GAS - SEQ ID NO: 29 | GVYGMDVWGQGTTVT | SFNAPLTFGGGTKVE |
| | QQSFNAPLT - SEQ ID NO: | VSS - SEQ ID | IK - SEQ ID NO: |
| | 30 | NO: 31 | 32 |
| 2F7 | GFTFSDYY - SEQ ID NO: 33 | QVQLVESGGGLVKPG | DIVMTQSPDSLAVSL |
| | IDSSAGAI - SEQ ID NO: | GSLRLSCVASGFTFS | GERATINCKSSQNIL |
| | 34 | DYYMNWIRLPPGRGL | YTSNNKNYLGWYQQK |
| | ARERRDGHKIFDW - SEQ ID | QWISYIDSSAGAIYY | AGQPPKLLIYWSSTR |
| | NO: 35 | ADSVKGRFTVSRDDA | NSGVPDRFSGSGSGT |
| | QNILYTSNNKNY - SEQ ID | KNSLYLQMNSLRADD | DETLTISSLQAEDVA |
| | NO: 36 | TAVYYCARERRDGHK | VYYCHQYYSSPYTFG |
| | WSS - SEQ ID NO: 37 | IFDWWGQGTLVTVSS - | QGTNLEIK - SEQ |
| | HQYYSSPYT - SEQ ID NO: 38 | SEQ ID NO: 39 | ID NO: 40 |
| 2F11 | GYSFTNYW - SEQ ID NO: | EVQLVQSGAEVKKPG | DIQMTQSPSSLSASV |
| | 41 | ESLKISCKGSGYSFT | GDRVTITCRASQSVN |
| | IYPGDSDT - SEQ ID NO: | NYWIGWVRQMPGKGL | NFLNWYQQKPGTAPK |
| | 42 | EWMGIIYPGDSDTRY | LLIYAASSLQGGVPS |
| | ATHARSWNYVAY - SEQ ID | SPSFQGQVTLSADKS | RFSGSGSGTDFTLTI |
| | NO: 43 | ISTAYLQWNSLKASD | SSLQPEDFATYYCQQ |
| | QSVNNF - SEQ ID NO: 44 | TAMYYCATHARSWNY | SNTTPWTFGQGTKVE |
| | AAS - SEQ ID NO: 45 | VAYWGQGTLVTVAS - | IK - SEQ ID NO: |
| | QQSNTTPWT - SEQ ID NO: 46 | SEQ ID NO: 47 | 48 |
| 2H1 | GFTFSDYY - SEQ ID NO: 49 | QVQLVESGGDLVKPG | DIVMTQSPDSLAVSL |
| | IDSSAGAI - SEQ ID NO: | GSLRLSCVASGFTFS | GERATINCKSSQNIL |
| | 50 | DYYMNWIRLPPGKGL | YSSNNKNYLAWYQQK |
| | ARERRDGHKIFDC - SEQ ID | EWISYIDSSAGAIYY | TGQPPKLLIFWSSTR |
| | NO: 51 | ADSVKGRFTVSRDDA | ESGVPDRFSGSGSGT |
| | QNILYSSNNKNY - SEQ ID | KNSLFLHMNSLRADD | DFTLTISSLQAEDVA |
| | NO: 52 | TAVYYCARERRDGHK | VYYCQQYYSSPYNFG |
| | WSS - SEQ ID NO: 53 | IFDCWGPGTLVTVSS - | RGTTLEIK - SEQ |
| | QQYYSSPYN - SEQ ID NO: 54 | SEQ ID NO: 55 | ID NO: 56 |
| 4E6 | GFTEDDYA - SEQ ID NO: 57 | EVQLVESGGDLVQPG | DIVMTQSPDSLAVSL |
| | ISWSGVTM - SEQ ID NO: | RSLRLSCAASGFTFD | GERATINCKSSQSVL |
| | 58 | DYAMHWVRQGPGKGL | YKSNNKNYLDWYQQK |
| | ARGDGTNAFDI - SEQ ID | EWVSGISWSGVTMGY | PGQPPKLLIYWASSR |
| | NO: 59 | ADSVKGRFTISRDNA | ESGVPDRESGSGSGT |
| | QSVLYKSNNKNY - SEQ ID | KNSLYLRMNSLRADD | DFTLTISSLQAEDVA |
| | NO: 60 | TAFYYCARGDGTNAF | VYYCQQYYSVPLTFG |
| | WAS - SEQ ID NO: 61 | DIWGHGTMVTVSS - | GGTKVEIK - SEQ |
| | QQYYSVPLT - SEQ ID NO: 62 | SEQ ID NO: 63 | ID NO: 6 |
| 4F7 | GFTFSDYY - SEQ ID NO: 65 | QVQLVESGGDLVKPG | DIVMTQSPDSLAVSL |
| | IDSSAGAI - SEQ ID NO: 66 | GSLRLSCVASGFTFS | GERATINCKSSQNIL |
| | ARERRDGHKIFDC - SEQ ID | DYYMNWIRLPPGKGL | YSSNNKNYLAWYQQK |
| | NO: 67 | EWISYIDSSAGAIYY | AGQPPKLLIFWSSTR |
| | QNILYSSNNKNY - SEQ ID | ADSVKGRFTVSRDDA | ESGVPDRESGSGSGT |
| | NO: 68 | KNSLFLHMNNLRADD | DFTLTISSLQAEDVA |
| | WSS - SEQ ID NO: 69 | TAVYYCARERRDGHK | VYYCQQYYSSPYNFG |
| | QQYYSSPYN - SEQ ID NO: | IFDCWGPGTLVTVSS - | RGTTLEIK - SEQ |
| | 70 | SEQ ID NO: 71 | ID NO: 72 |
| 5A8 | GFTESDYY - SEQ ID NO: 73 | QVQLVESGGDLVKPG | DIVMTQSPDSLAVSL |
| | IDSSAGAI - SEQ ID NO: 74 | GSLRLSCVASGFTFS | GERATINCKSSQNIL |
| | ARERRDGHKIFDY - SEQ ID | DYYMNWIRLPPGRGL | YSSNNKNYLAWYQQK |
| | NO: 75 | EWISYIDSSAGAIYY | AGQPPKLLIFWSSTR |
| | QNILYSSNNKNY - SEQ ID | ADSVKGRFTVSRDDA | ESGVPDRFSGSGSGT |
| | NO: 76 | KKSLFLYMNSLRADD | DFTLTISSLQAEDVA |
| | WSS - SEQ ID NO: 77 | TAVYYCARERRDGHK | VYYCHQYYSSPYNFG |
| | HQYYSSPYN - SEQ ID NO: | IFDYWGPGTLVTVSS - | RGTTLEIK - SEQ |
| | 78 | SEQ ID NO: 79 | ID NO: 80 |
| 6E1 | GFTFSSFA - SEQ ID NO: | EVQLLESGGGLVQPG | DIQMTQSPSSLSASV |
| | 81 | GSLRLSCAASGFTFS | GGRVTITCRTSQSIS |
| | ISGSGGST - SEQ ID NO: | SFAMSWVRQAPGKGL | GYLNWYQQKAGKAPK |
| | 82 | EWVSGISGSGGSTYY | LLIYATSNLQSGVPS |
| | AKDKRNWNYGIDSFDF - SEQ | ADSVKGRFTISRDNS | RFTGSGSGTDFTLTI |
| | ID NO: 83 | KNTLYLQMNSLRAED | SSLQPEDFAAYYCHQ |
| | QSISGY - SEQ ID NO: 84 | TAIYYCAKDKRNWNY | SHSPPFTFGPGTKVA |
| | ATS - SEQ ID NO: 85 | GIDSFDFWGQGTMVT | FK - SEQ ID NO: |
| | HQSHSPPFT - SEQ ID NO: | VSS - SEQ ID | 88 |
| | 86 | NO: 87 | |
| 6H12 | GFTFSSYE - SEQ ID NO: | EVQLVESGGGLVQPG | DIQLTQSPSFLSASV |
| | 89 | GSLRLSCAASGFTFS | GDRVTITCWASQDIS |
| | ISSGGSSI - SEQ ID NO: | SYEMNWVRQAPGKGL | SYLAWYQQKPGKAPK |
| | 90 | EWVSYISSGGSSIKY | LLIYAASTLQSGVPS |
| | ARVGGSYYYSYAMDV - SEQ | ADSVRGRFAFSRDNA | RFTGSGSGTEFTLTI |
| | ID NO: 91 | ENSVHLQMNSLRAED | SSLQPEDFATYYCQQ |
| | QDISSY - SEQ ID NO: 92 | TAVYYCARVGGSYYY | PHNSPFSFGPGTKVD |
| | AAS - SEQ ID NO: 93 | SYAMDVWGQGTAVTV | VK - SEQ ID NO: |
| | QQPHNSPFS - SEQ ID NO: | SS - SEQ ID NO: | 96 |
| | 94 | 95 | |
| 7A4 | GFTFSDYY - SEQ ID NO: 97 | QVQLVESGGDLVKPG | DIVMTQSPDSLAVSL |
| | IDSSAGAI - SEQ ID NO: | GSLRLSCVASGFTFS | GERATINCKSSQNIL |
| | 98 | DYYMNWIRLPPGKGL | YSSNNKNYLAWYQQK |
| | VRERRDGHKIFDY - SEQ ID | EWISYIDSSAGAIYY | AGQPPKLLIFWSSIR |
| | NO: 99 | ADSVKGRFTVSRDDA | ESGVPDRFSGSGSGT |
| | QNILYSSNNKNY - SEQ ID | KNSLFLHMNSLRADD | DFTLTISSLQAEDVA |
| | NO: 100 | TAVYYCVRERRDGHK | VYYCQQYYSSPYNFG |
| | WSS - SEQ ID NO: 101 | IFDYWGPGTLVTVSS - | RGTTLEIK - SEQ |
| | QQYYSSPYN - SEQ ID NO: | SEQ ID NO: | ID NO: 104 |
| | 102 | 103 | |
| 7E5 | GFTFSGFA - SEQ ID NO: | EVQLLESGGGLIQPG | DIQMTQSPSSLSASV |
| | 105 | GSLRLSCAASGFTES | GDRVTITCRASQSIS |
| | ISGSGDIT - SEQ ID NO: | GFAMSWVRQAPGKGL | TYLNWYQQKPGKAPK |
| | 106 | EWVSGISGSGDITYY | FLIYGASSLQSGVPS |
| | GKDPYNWNHGVYGMDV - SEQ | GDSVKGRFTISRDNS | RFSGSGSGTDFSLTI |
| | ID NO: 107 | KSTLYLQMNSLRDAD | SSLHPGDFATYYCQQ |
| | QSISTY - SEQ ID NO: 108 | TAVYYCGKDPYNWNH | SYNIPLTFGGGTKVE |
| | GAS - SEQ ID NO: 109 | GVYGMDVWGQGTTVT | IK - SEQ ID NO: |
| | QQSYNIPLT - SEQ ID NO: | VSS - SEQ ID | 112 |
| | 110 | NO: 111 | |
| 7H8 | GFAFHTYD - SEQ ID NO: | EVQLLESGGGLVQPG | DIQMTQSPSTLSASV |
| | 113 | GSLRLSCAASGFAFH | GDRVTITCRASQSIS |
| | VVGSGINT - SEQ ID NO: | TYDMTWVRQAPGKGL | NWLAWYQQKPGKAPK |
| | 114 | EWVSGVVGSGINTYY | LLIYKASSLESGVPS |
| | AKDSSSWFSLHY - SEQ ID | ADSVKGRFTISRDNS | RFSGSGSGTDFTLTI |
| | NO: 115 | KSTLYLQMNSLRAED | SSLQPDDFASYYCQQ |
| | QSISNW - SEQ ID NO: 116 | TAVYYCAKDSSSWFS | YRSYAYTFGQGTKLE |
| | KAS - SEQ ID NO: 117 | LHYWGQGTLVTVSS - | IT - SEQ ID NO: |
| | QQYRSYAYT - SEQ ID NO: | SEQ ID NO: | 120 |
| | 118 | 119 | |
| 8A11 | GFTFSDYY - SEQ ID NO: 121 | QVQLVESGGDLVKPG | DIVMTQSPDSLAVSL |
| | IDSSAGAT - SEQ ID NO: | GSLRLSCVASGFTFS | GERATINCKSSQNIL |
| | 122 | DYYMNWIRLPPGKGL | YSSNNKNYLAWYQQK |
| | ARERRSGHKIFDC - SEQ ID | EWISYIDSSAGATYY | AGQPPKLLIFWSSTR |
| | NO: 123 | ADSVKGRFTVSRDDA | ESGVPDRFSGSGSGT |
| | QNILYSSNNKNY - SEQ ID | KNSLFLHMNSLRAED | DFTLTISSLQAEDVA |
| | NO: 124 | TAVYYCARERRSGHK | VYYCQQYYSSPYNFG |
| | WSS - SEQ ID NO: 125 | IFDCWGPGTLVTVSS - | RGTTLEIK - SEQ |
| | QQYYSSPYN - SEQ ID NO: | SEQ ID NO: | ID NO: 128 |
| | 126 | 127 | |
| 8D10 | GFTESDYY - SEQ ID NO: 129 | QVHLVESGGDLVKPG | DIVMTQSPDSLAVSL |
| | IDSSAGAI - SEQ ID NO: | GSLRLSCVASGFTFS | GERATINCKSSQNIL |
| | 130 | DYYMNWIRLPPGKGL | YSSNNKNYLAWYQQK |
| | ARERRDGHKIFDY - SEQ ID | EWISYIDSSAGAIYY | AGQPPKLLIFWSSTR |
| | NO: 131 | ADSVKGRFTVSRDDA | ESGVPDRFSGSGSGT |
| | QNILYSSNNKNY - SEQ ID | KNSMFLHMNSLRADD | DFTLTISSLQAEDVA |
| | NO: 132 | TAVYYCARERRDGHK | VYYCQQYYSSPYNFG |
| | WSS - SEQ ID NO: 133 | IFDYWGPGTLVTVSS - | RGTTLEIK - SEQ |
| | QQYYSSPYN - SEQ ID NO: | SEQ ID NO: | ID NO: 136 |
| | 134 | 135 | |
| 9B4 | GFTFSDYY - SEQ ID NO: 137 | QVQLVESGGDLVKPG | DIVMTQSPDSLALSL |
| | IDSSAGAI - SEQ ID NO: | GSLRLSCVASGFTFS | GERATINCKSSQNIL |
| | 138 | DYYMNWIRLPPGKGL | YSSNNKNYLACYQQK |
| | ARERREGHKIFDF - SEQ ID | EWISYIDSSAGAIYY | AGQPPKLLIFWSSTR |
| | NO: 139 | ADSVKGRFTVSRDDA | ESGVPDRFSGSGSGT |
| | QNILYSSNNKNY - SEQ ID | KNSLFLHMNSLRADD | DFTLTISSLQAEDVA |
| | NO: 140 | TAVYYCARERREGHK | VYYCQQYYSSPYNFG |
| | WSS - SEQ ID NO: 141 | IFDFWGPGTLVTVSS - | RGTTLEIK - SEQ |
| | QQYYSSPYN - SEQ ID NO: | SEQ ID NO: | ID NO: 144 |
| | 142 | 143 | |
| 9G9 | GFTFHNYA - SEQ ID NO: | EVQLLESGGGLVQPG | DIQMTQSPSSLSASV |
| | 145 | GSLRLSCAASGFTFH | GDRVTITCRASQSIG |
| | ISGSGGTA - SEQ ID NO: | NYAMSWVRQAPGKGL | SFLIWYQQKPGKAPK |
| | 146 | EWVSGISGSGGTAYY | LLIYAASSLQSGVPS |
| | AKDRFLEWVEGFDS - SEQ | ADSVKGRFTISRDNS | RFSGSGSGTDFTLII |
| | ID NO: 147 | KNTLYLQMNSLRADD | SSLQPEDFATYYCQQ |
| | QSIGSF - SEQ ID NO: 148 | TALYYCAKDRFLEWV | SYNTPLTFGGGTKVE |
| | AAS - SEQ ID NO: 149 | EGFDSWGQGTLVTVS | IK - SEQ ID NO: |
| | QQSYNTPLT - SEQ ID NO: | S - SEQ ID NO: | 152 |
| | 150 | 151 | |
| 9H9 | GFTFSSYE - SEQ ID NO: | EVQLVESGGGLVQPG | DIQLTQSPSFLSASV |
| | 153 | GSLRLSCAASGFTES | GDRVTITCWASQDIS |
| | ISSGGSSI - SEQ ID NO: | SYEMNWVRQAPGKGL | SYLAWYQQKPGKAPK |
| | 154 | EWVSYISSGGSSIKY | LLIYAASTLQSGVPS |
| | ARVGGSYYYSYAMDV - SEQ | ADSVRGRFTFSRDNA | RFSGSGSGTEFTLTI |
| | ID NO: 155 | ENSVHLQMNSLRGED | SSLQPEDFATYYCQQ |
| | QDISSY - SEQ ID NO: 156 | TAVYYCARVGGSYYY | PNNYPFSFGPGTKVD |
| | AAS - SEQ ID NO: 157 | SYAMDVWGQGTAVTV | VK - SEQ ID NO: |
| | QQPNNYPFS - SEQ ID NO: | SS - SEQ ID NO: | 160 |
| | 158 | 159 | |
| 11D12 | GFTFSSYE - SEQ ID NO: | EVQLVESGGGLVQPG | DIQLTQSPSFLSASV |
| | 161 | GSLRLSCAASGFTFS | GDRVTITCWASQDIS |
| | ISSGGSSI - SEQ ID NO: | SYEMNWVRQAPGKGL | SYLAWYQQKPGKAPK |
| | 162 | EWVSYISSGGSSIKY | LLIYAASTLQSGVPS |
| | ARVGGSYYYSYAMDV - SEQ | ADSVRGRFTFSRDNA | RFSGSGSGTEFTLTI |
| | ID NO: 163 | ENSVFLQMNSLRGED | SSLQPEDFATYYCQQ |
| | QDISSY - SEQ ID NO: 164 | TAVYYCARVGGSYYY | PNNYPFSFGPGTKVD |
| | AAS - SEQ ID NO: 165 | SYAMDVWGQGTAVTV | VK - SEQ ID NO: |
| | QQPNNYPES - SEQ ID NO: | SS - SEQ ID NO: | 168 |
| | 166 | 167 | |
| 14F5 | GFTFSSFA - SEQ ID NO: | EVQLLESGGGLVQPG | DIQMTQSPSSLSASV |
| | 169 | GSLRLSCAVSGFTFS | GDRVTITCRASQSIG |
| | ISASGGTT - SEQ ID NO: | SFAMSWVRQAPGKGL | RFLIWYQQKPGKAPK |
| | 170 | EWVSGISASGGTTNY | LLLYATSSLQSGVPA |
| | AKDRFLEWVEGFDP - SEQ | ADSVKGRFTISRDNS | RFTGSGSGTDFTLTI |
| | ID NO: 171 | KNTLYLQMSSLRAED | GSLQPEDFATYYCQQ |
| | QSIGRF - SEQ ID NO: 172 | TAEYYCAKDRFLEWV | SYITPLTFGGGTKVE |
| | ATS - SEQ ID NO: 173 | EGFDPWGQGILVTVS | IK - SEQ ID NO: |
| | QQSYITPLT - SEQ ID NO: | P - SEQ ID NO: | 176 |
| | 174 | 175 | |
| 15C10 | GFTFSSFA - SEQ ID NO: | EVQLLESGGGLVQPG | DIQMTQSPSSLSASV |
| | 177 | GSLRLSCAASGFTFS | GDRVTITCRASQSIG |
| | ISASGGTT - SEQ ID NO: | SFAMSWVRQAPGKGL | RFLIWYQQKPGKAPK |
| | 178 | EWVSGISASGGTTNY | LLLYATSSLQSGVPA |
| | AKDRFLEWVEGFDP - SEQ | ADSVKGRFTISRDNS | RFTGSGSGTDFTLTI |
| | ID NO: 179 | KNTLYLQMSSLRAED | GSLQPEDFATYYCQQ |
| | QSIGRF - SEQ ID NO: 180 | TAEYYCAKDRFLEWV | SYITPLTFGGGTKVE |
| | ATS - SEQ ID NO: 181 | EGFDPWGQGILVTVS | IK - SEQ ID NO: |
| | QQSYITPLT - SEQ ID NO: | P - SEQ ID NO: | 184 |
| | 182 | 183 | |

TABLE 2
Amino acid sequences of antibody framework regions.

| Antibody | |
|---|---|
| Clone | Sequence |
| 1E2 | HFR1 - QVQLVESGGDLVKPGGSLRLSCVAS - SEQ ID NO: 185 |
| | HFR2 - MNWIRLPPGKGLEWVSY - SEQ ID NO: 186 |
| | HFR3 - YYADSVRGRFTVSRDDAKNSLFLHMNSLRADDTAVYYC - SEQ ID |
| | NO: 187 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 LAWYQQKAGQPPKLLIF - SEQ ID NO: 190 |
| | LFR3 - TRESGVPDRFSGSGSGTDFTLTINSLQAEDVAVYYC - SEQ ID NO: |
| | 191 |
| | LFR4 - FGRGTTLEIK - SEQ ID NO: 192 |
| 1H10 | HFR1 - QVHLVESGGDLVKPGGSLRLSCVAS - SEQ ID NO: 193 |
| | HFR2 - MNWIRMPPGKGLEWISY - SEQ ID NO: 194 |
| | HFR3 - YYADSVKGRFTVSRDDAKNSLFLHMNNLRADDTAVYYC - SEQ ID |
| | NO: 195 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKAGQPPKLLIF - SEQ ID NO: 190 |
| | LFR3 · TRESGVPDRESGSGSGTDFTLTISSLQAEDVAVYYC - SEQ ID NO: |
| | 196 |
| | LFR4 - FGRGTTLEIK - SEQ ID NO: 192 |
| 2A10 | HFR1 - EVQLVQSGAEVKKPGESLKISCKGS - SEQ ID NO: 197 |
| | HFR2 IGWVRQMPGKGLEWMGI - SEQ ID NO: 198 |
| | HFR3 - RYSPSFQGQVTISADKSISTAYLQWSSLKASDTAMYYC - SEQ ID |
| | NO: 199 |
| | HFR4 - WGQGTLVTVSS - SEQ ID NO: 200 |
| | LFR1 - DIQMTQSPSSLSASVGDRLTITCRAS - SEQ ID NO: 201 |
| | LFR2 LNWYQQNPGKAPKVLIY - SEQ ID NO: 202 |
| | LFR3 SLESGVPSRFSGSGSGTDFTLTISSLQPEDFATYYC - SEQ ID NO: |
| | 203 |
| | LFR4 - FGQGTKVEIK - SEQ ID NO: 204 |
| 2B9 | HFR1 - EVQLLESGGGLAQPGKSLRLSCAAS - SEQ ID NO: 205 |
| | HFR2 - MSWVRQAPGKGLEWVSG - SEQ ID NO: 206 |
| | HFR3 - YYADSVKGRFTISRDNSKSTLYLQMNSLGAEDTAVYYC - SEQ ID |
| | NO: 207 |
| | HFR4 - WGQGTTVTVSS - SEQ ID NO: 208 |
| | LFR1 DIQMTQSPSSLSASVGDRVTITCRAS - SEQ ID NO: 209 |
| | LFR2 LNWYQQKPGKAPKFLIY - SEQ ID NO: 210 |
| | LFR3 - SLQSGVPSRFSGSGCGXDFTLTISSLQREDFAAYYC - SEQ ID NO: |
| | 211 |
| | LFR4 - FGGGTKVEIK - SEQ ID NO: 212 |
| 2F7 | HFR1 - QVQLVESGGGLVKPGGSLRLSCVAS - SEQ ID NO: 213 |
| | HFR2 - MNWIRLPPGRGLQWISY - SEQ ID NO: 214 |
| | HFR3 - YYADSVKGRFTVSRDDAKNSLYLQMNSLRADDTAVYYC - SEQ ID |
| | NO: 215 |
| | HFR4 - WGQGTLVTVSS - SEQ ID NO: 200 |
| | LFR1 - DIVMTQSPDSLAVSLGERATINCKSS - SEQ ID NO: 189 |
| | LFR2 - LGWYQQKAGQPPKLLIY - SEQ ID NO: 216 |
| | LFR3 - TRNSGVPDRFSGSGSGTDFTLTISSLQAEDVAVYYC - SEQ ID NO: |
| | 217 |
| | LFR4 - FGQGTNLEIK - SEQ ID NO: 218 |
| 2F11 | HFR1 - EVQLVQSGAEVKKPGESLKISCKGS - SEQ ID NO: 197 |
| | HFR2 IGWVRQMPGKGLEWMGI - SEQ ID NO: 198 |
| | HFR3 - RYSPSFQGQVTLSADKSISTAYLQWNSLKASDTAMYYC - SEQ ID |
| | NO: 219 |
| | HFR4 - WGQGTLVTVAS - SEQ ID NO: 220 |
| | LFR1 - DIQMTQSPSSLSASVGDRVTITCRAS - SEQ ID NO: 209 |
| | LFR2 - LNWYQQKPGTAPKLLIY - SEQ ID NO: 221 |
| | LFR3 - SLQGGVPSRFSGSGSGTDFTLTISSLQPEDFATYYC - SEQ ID NO: |
| | 222 |
| | LFR4 - FGQGTKVEIK - SEQ ID NO: 204 |
| 2H1 | HFR1 - QVQLVESGGDLVKPGGSLRLSCVAS - SEQ ID NO: 185 |
| | HFR2 - MNWIRLPPGKGLEWISY - SEQ ID NO: 223 |
| | HFR3 - YYADSVKGRFTVSRDDAKNSLFLHMNSLRADDTAVYYC - SEQ ID |
| | NO: 224 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKTGQPPKLLIF - SEQ ID NO: 225 |
| | LFR3 - TRESGVPDRFSGSGSGTDFTLTISSLQAEDVAVYYC - SEQ ID NO: |
| | 196 |
| | LFR4 - FGRGTTLEIK - SEQ ID NO: 192 |
| 4E6 | HFR1 - EVQLVESGGDLVQPGRSLRLSCAAS - SEQ ID NO: 226 |
| | HFR2 - MHWVRQGPGKGLEWVSG - SEQ ID NO: 227 |
| | HFR3 - GYADSVKGRFTISRDNAKNSLYLRMNSLRADDTAFYYC - SEQ ID |
| | NO: 228 |
| | HFR4 - WGHGTMVTVSS - SEQ ID NO: 229 |
| | LFR1 - DIVMTQSPDSLAVSLGERATINCKSS - SEQ ID NO: 189 |
| | LFR2 - LDWYQQKPGQPPKLLIY - SEQ ID NO: 230 |
| | LFR3 SRESGVPDRFSGSGSGTDFTLTISSLQAEDVAVYYC - SEQ ID NO: |
| | 231 |
| | LFR4 - FGGGTKVEIK - SEQ ID NO: 212 |
| 4F7 | HFR1 - QVQLVESGGDLVKPGGSLRLSCVAS - SEQ ID NO: 185 |
| | HFR2 - MNWIRLPPGKGLEWISY - SEQ ID NO: 223 |
| | HFR3 - YYADSVKGRFTVSRDDAKNSLFLHMNNLRADDTAVYYC - SEQ ID |
| | NO: 195 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKAGQPPKLLIF - SEQ ID NO: 190 |
| | LFR3 TRESGVPDRESGSGSGTDETLTISSLQAEDVAVYYC - SEQ ID NO: |
| | 196 |
| | LFR4 - FGRGTTLEIK - SEQ ID NO: 192 |
| 5A8 | HFR1 - QVQLVESGGDLVKPGGSLRLSCVAS - SEQ ID NO: 185 |
| | HFR2 - MNWIRLPPGRGLEWISY - SEQ ID NO: 232 |
| | HFR3 - YYADSVKGRFTVSRDDAKKSLFLYMNSLRADDTAVYYC - SEQ ID |
| | NO: 233 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKAGQPPKLLIF - SEQ ID NO: 190 |
| | LFR3 - TRESGVPDRFSGSGSGTDFTLTISSLQAEDVAVYYC - SEQ ID NO: |
| | 196 |
| | LFR4 - FGRGTTLEIK - SEQ ID NO: 192 |
| 6E1 | HFR1 - EVQLLESGGGLVQPGGSLRLSCAAS - SEQ ID NO: 234 |
| | HFR2 - MSWVRQAPGKGLEWVSG - SEQ ID NO: 206 |
| | HFR3 - YYADSVKGRFTISRDNSKNTLYLQMNSLRAEDTAIYYC - SEQ ID |
| | NO: 235 |
| | HFR4 - WGQGTMVTVSS - SEQ ID NO: 236 |
| | LFR1 - DIQMTQSPSSLSASVGGRVTITCRTS - SEQ ID NO: 237 |
| | LFR2 - LNWYQQKAGKAPKLLIY - SEQ ID NO: 238 |
| | LFR3 NLQSGVPSRFTGSGSGTDFTLTISSLQPEDFAAYYC - SEQ ID NO: |
| | 239 |
| | LFR4 - FGPGTKVAFK - SEQ ID NO: 240 |
| 6H12 | HFR1 EVQLVESGGGLVQPGGSLRLSCAAS - SEQ ID NO: 241 |
| | HFR2 MNWVRQAPGKGLEWVSY - SEQ ID NO: 242 |
| | HFR3 - KYADSVRGRFAFSRDNAENSVHLQMNSLRAEDTAVYYC - SEQ ID |
| | NO: 243 |
| | HFR4 WGQGTAVTVSS - SEQ ID NO: 244 |
| | LFR1 - DIQLTQSPSFLSASVGDRVTITCWAS - SEQ ID NO: 245 |
| | LFR2 - LAWYQQKPGKAPKLLIY - SEQ ID NO: 246 |
| | LFR3 - TLQSGVPSRFTGSGSGTEFTLTISSLQPEDFATYYC - SEQ ID NO: |
| | 247 |
| | LFR4 - FGPGTKVDVK - SEQ ID NO: 248 |
| 7A4 | HFR1 - QVQLVESGGDLVKPGGSLRLSCVAS - SEQ ID NO: 185 |
| | HFR2 - MNWIRLPPGKGLEWISY - SEQ ID NO: 223 |
| | HFR3 - YYADSVKGRFTVSRDDAKNSLFLHMNSLRADDTAVYYC - SEQ ID |
| | NO: 224 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 LAWYQQKAGQPPKLLIF - SEQ ID NO: 190 |
| | LFR3 - IRESGVPDRFSGSGSGTDFTLTISSLQAEDVAVYYC - SEQ ID NO: |
| | 249 |
| | LFR4 - FGRGTTLEIK - SEQ ID NO: 192 |
| 7E5 | HFR1 - EVQLLESGGGLIQPGGSLRLSCAAS - SEQ ID NO: 250 |
| | HFR2 MSWVRQAPGKGLEWVSG - SEQ ID NO: 206 |
| | HFR3 - YYGDSVKGRFTISRDNSKSTLYLQMNSLRDADTAVYYC - SEQ ID |
| | NO: 251 |
| | HFR4 - WGQGTTVTVSS - SEQ ID NO: 208 |
| | LFR1 - DIQMTQSPSSLSASVGDRVTITCRAS - SEQ ID NO: 209 |
| | LFR2 - LNWYQQKPGKAPKFLIY - SEQ ID NO: 210 |
| | LFR3 - SLQSGVPSRFSGSGCGTDESLTISSXHPGDFATYYC - SEQ ID NO: |
| | 252 |
| | LFR4 - FGGGTKVEIK - SEQ ID NO: 212 |
| 7H8 | HFR1 - EVQLLESGGGLVQPGGSLRLSCAAS - SEQ ID NO: 234 |
| | HFR2 - MTWVRQAPGKGLEWVSG - SEQ ID NO: 253 |
| | HFR3 - YYADSVKGRFTISRDNSKSTLYLQMNSLRAEDTAVYYC - SEQ ID |
| | NO: 254 |
| | HFR4 - WGQGTLVTVSS - SEQ ID NO: 200 |
| | LFR1 - DIQMTQSPSTLSASVGDRVTITCRAS - SEQ ID NO: 255 |
| | LFR2 LAWYQQKPGKAPKLLIY - SEQ ID NO: 246 |
| | LFR3 - SLESGVPSRFSGSGSGTDFTLTISSLQPDDFASYYC - SEQ ID NO: |
| | 256 |
| | LFR4 - FGQGTKLEIT - SEQ ID NO: 257 |
| 8A11 | HFR1 - QVQLVESGGDLVKPGGSLRLSCVAS - SEQ ID NO: 185 |
| | HFR2 - MNWIRLPPGKGLEWISY - SEQ ID NO: 223 |
| | HFR3 - YYADSVKGRFTVSRDDAKNSLFLHMNSLRAEDTAVYYC - SEQ ID |
| | NO: 258 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKAGQPPKLLIF - SEQ ID NO: 190 |
| | LFR3 - TRESGVPDRESGSGSGTDFTLTISSLQAEDVAVYYC - SEQ ID NO: |
| | 196 |
| | LFR4 - FGRGTTLEIK - SEQ ID NO: 192 |
| 8D10 | HFR1 QVHLVESGGDLVKPGGSLRLSCVAS - SEQ ID NO: 193 |
| | HFR2 - MNWIRLPPGKGLEWISY - SEQ ID NO: 223 |
| | HFR3 - YYADSVKGRFTVSRDDAKNSMFLHMNSLRADDTAVYYC - SEQ ID |
| | NO: 259 |
| | HFR4 - WGPGTLVTVSS - SEQ ID NO: 188 |
| | LFR1 - DIVMTQSPDSLAVSLGERATINCKSS - SEQ ID NO: 189 |
| | LFR2 - LAWYQQKAGQPPKLLIF - SEQ ID NO: 190 |
| | LFR3 TRESGVPDRFSGSGSGTDFTLTISSLQAEDVAVYYC - SEQ ID NO: |
| | 196 |
| | LFR4 - FGRGTTLEIK - SEQ ID NO: 192 |
| 9B4 | HFR1 - QVQLVESGGDLVKPGGSLRLSCVAS - SEQ ID NO: 185 |
| | HFR2 - MNWIRLPPGKGLEWISY - SEQ ID NO: 223 |
| | HFR3 - YYADSVKGRFTVSRDDAKNSLFLHMNSLRADDTAVYYC - SEQ ID |
| | NO: 224 |
| | HER4 WGPGTLVTVSS - SEQ ID NO: 188 |
| | LFR1 - DIVMTQSPDSLALSLGERATINCKSS - SEQ ID NO: 260 |
| | LFR2 - LACYQQKAGQPPKLLIF - SEQ ID NO: 261 |
| | LFR3 - TRESGVPDRESGSGSGTDFTLTISSLQAEDVAVYYC - SEQ ID NO: |
| | 196 |
| | LFR4 - FGRGTTLEIK - SEQ ID NO: 192 |
| 9G9 | HFR1 - EVQLLESGGGLVQPGGSLRLSCAAS - SEQ ID NO: 234 |
| | HFR2 - MSWVRQAPGKGLEWVSG - SEQ ID NO: 206 |
| | HFR3 - YYADSVKGRFTISRDNSKNTLYLQMNSLRADDTALYYC - SEQ ID |
| | NO: 262 |
| | HFR4 - WGQGTLVTVSS - SEQ ID NO: 200 |
| | LFR1 - DIQMTQSPSSLSASVGDRVTITCRAS - SEQ ID NO: 209 |
| | LFR2 - LIWYQQKPGKAPKLLIY - SEQ ID NO: 263 |
| | LFR3 - SLQSGVPSRFSGSGSGTDFTLIISSLQPEDFATYYC - SEQ ID NO: |
| | 264 |
| | LFR4 - FGGGTKVEIK - SEQ ID NO: 212 |
| 9H9 | HFR1 - EVQLVESGGGLVQPGGSLRLSCAAS - SEQ ID NO: 241 |
| | HFR2 - MNWVRQAPGKGLEWVSY - SEQ ID NO: 242 |
| | HFR3 - KYADSVRGRFTFSRDNAENSVHLQMNSLRGEDTAVYYC - SEQ ID |
| | NO: 265 |
| | HFR4 - WGQGTAVTVSS - SEQ ID NO: 244 |
| | LFR1 - DIQLTQSPSFLSASVGDRVTITCWAS - SEQ ID NO: 245 |
| | LFR2 LAWYQQKPGKAPKLLIY - SEQ ID NO: 246 |
| | LFR3 - TLQSGVPSRFSGSGXGTEFTLTISSLQREDFATYYC - SEQ ID NO: |
| | 266 |
| | LFR4 - FGPGTKVDVK - SEQ ID NO: 248 |
| 11D12 | HFR1 - EVQLVESGGGLVQPGGSLRLSCAAS - SEQ ID NO: 241 |
| | HFR2 - MNWVRQAPGKGLEWVSY - SEQ ID NO: 242 |
| | HFR3 - KYADSVRGRFTFSRDNAENSVFLQMNSLRGEDTAVYYC - SEQ ID |
| | NO: 267 |
| | HFR4 - WGQGTAVTVSS - SEQ ID NO: 244 |
| | LFR1 - DIQLTQSPSFLSASVGDRVTITCWAS - SEQ ID NO: 245 |
| | LFR2 LAWYQQKPGKAPKLLIY - SEQ ID NO: 246 |
| | LFR3 - TLQSGVPSRFSGSGSGTEFTLTISSLQPEDFATYYC - SEQ ID NO: |
| | 268 |
| | LFR4 - FGPGTKVDVK - SEQ ID NO: 248 |
| 14F5 | HFR1 - EVQLLESGGGLVQPGGSLRLSCAVS - SEQ ID NO: 269 |
| | HFR2 - MSWVRQAPGKGLEWVSG - SEQ ID NO: 206 |
| | HFR3 - NYADSVKGRFTISRDNSKNTLYLQMSSLRAEDTAEYYC - SEQ ID |
| | NO: 270 |
| | HFR4 - WGQGILVTVSP - SEQ ID NO: 271 |
| | LFR1 - DIQMTQSPSSLSASVGDRVTITCRAS - SEQ ID NO: 209 |
| | LFR2 LIWYQQKPGKAPKLLLY - SEQ ID NO: 272 |
| | LFR3 - SLQSGVPARFTGSGSGTDFTLTIGSLQPEDFATYYC - SEQ ID NO: |
| | 273 |
| | LFR4 - FGGGTKVEIK - SEQ ID NO: 212 |
| 15C10 | HFR1 - EVQLLESGGGLVQPGGSLRLSCAAS - SEQ ID NO: 234 |
| | HFR2 MSWVRQAPGKGLEWVSG - SEQ ID NO: 206 |
| | HFR3 - NYADSVKGRFTISRDNSKNTLYLQMSSLRAEDTAEYYC - SEQ ID |
| | NO: 270 |
| | HER4 - WGQGILVTVSP - SEQ ID NO: 271LFR1 - |
| | DIQMTQSPSSLSASVGDRVTITCRAS - SEQ ID NO: 209 |
| | LFR2 - LIWYQQKPGKAPKLLLY - SEQ ID NO: 272 |
| | LFR3 - SLQSGVPARFTGSGSGTDFTLTIGSLQPEDFATYYC - SEQ ID NO: |
| | 273 |
| | LFR4 - FGGGTKVEIK - SEQ ID NO: 212 |

TABLE 3
Sequences of antibody heavy chain constant regions by isotype.

| Heavy | | |
|---|---|---|
| Chain | Accession | |
| Isotype | No. | Sequence |
| Human | P01857 | ASTKGPSVFPLAPSSKSTSGGTAALGCLVKDYFPEPVTVSWNSGALTSGVHT |
| IgG1 | | FPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK (SEQ ID NO: 274) |
| Human | P01859 | ASTKGPSVFPLAPCSRSTSESTAALGCLVKDYFPEPVTVSWNSGALTSGVHT |
| IgG2 | | FPAVLQSSGLYSLSSVVTVPSSNFGTQTYTCNVDHKPSNTKVDKTVERKCCV |
| | | ECPPCPAPPVAGPSVFLFPPKPKDTLMISRTPEVTCVVVDVSHEDPEVQFNW |
| | | YVDGVEVHNAKTKPREEQFNSTFRVVSVLTVVHQDWLNGKEYKCKVSNKGLP |
| | | APIEKTISKTKGQPREPQVYTLPPSREEMTKNQVSLTCLVKGFYPSDISVEW |
| | | ESNGQPENNYKTTPPMLDSDGSFFLYSKLTVDKSRWQQGNVFSCSVMHEALH |
| | | NHYTQKSLSLSPGK (SEQ ID NO: 275) |
| Human | P01860 | ASTKGPSVFPLAPCSRSTSGGTAALGCLVKDYFPEPVTVSWNSGALTSGVHT |
| IgG3 | | FPAVLQSSGLYSLSSVVTVPSSSLGTQTYTCNVNHKPSNTKVDKRVELKTPL |
| | | GDTTHTCPRCPEPKSCDTPPPCPRCPEPKSCDTPPPCPRCPEPKSCDTPPPC |
| | | PRCPAPELLGGPSVFLFPPKPKDTLMISRTPEVTCVVVDVSHEDPEVQFKWY |
| | | VDGVEVHNAKTKPREEQYNSTFRVVSVLTVLHQDWLNGKEYKCKVSNKALPA |
| | | PIEKTISKTKGQPREPQVYTLPPSREEMTKNQVSLTCLVKGFYPSDIAVEWE |
| | | SSGQPENNYNTTPPMLDSDGSFFLYSKLTVDKSRWQQGNIFSCSVMHEALHN |
| | | RFTQKSLSLSPGK (SEQ ID NO: 276) |
| Human | P01861 | ASTKGPSVFPLAPCSRSTSESTAALGCLVKDYFPEPVTVSWNSGALTSGVHT |
| IgG4 | | FPAVLQSSGLYSLSSVVTVPSSSLGTKTYTCNVDHKPSNTKVDKRVESKYGP |
| | | PCPSCPAPEFLGGPSVFLFPPKPKDTLMISRTPEVTCVVVDVSQEDPEVQFN |
| | | WYVDGVEVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDIAVE |
| | | WESNGQPENNYKTTPPVLDSDGSFFLYSRLTVDKSRWQEGNVFSCSVMHEAL |
| | | HNHYTQKSLSLSLGK (SEQ ID NO: 277) |
| Human | P01876 | ASPTSPKVFPLSLCSTQPDGNVVIACLVQGFFPQEPLSVTWSESGQGVTARN |
| IgA1 | | FPPSQDASGDLYTTSSQLTLPATQCLAGKSVTCHVKHYTNPSQDVTVPCPVP |
| | | STPPTPSPSTPPTPSPSCCHPRLSLHRPALEDLLLGSEANLTCTLTGLRDAS |
| | | GVTFTWTPSSGKSAVQGPPERDLCGCYSVSSVLPGCAEPWNHGKTFTCTAAY |
| | | PESKTPLTATLSKSGNTFRPEVHLLPPPSEELALNELVTLTCLARGFSPKDV |
| | | LVRWLQGSQELPREKYLTWASRQEPSQGTTTFAVTSILRVAAEDWKKGDTFS |
| | | CMVGHEALPLAFTQKTIDRLAGKPTHVNVSVVMAEVDGTCY (SEQ ID |
| | | NO: 278) |
| Human | P01877 | ASPTSPKVFPLSLDSTPQDGNVVVACLVQGFFPQEPLSVTWSESGQNVTARN |
| IgA2 | | FPPSQDASGDLYTTSSQLTLPATQCPDGKSVTCHVKHYTNSSQDVTVPCRVP |
| | | PPPPCCHPRLSLHRPALEDLLLGSEANLTCTLTGLRDASGATFTWTPSSGKS |
| | | AVQGPPERDLCGCYSVSSVLPGCAQPWNHGETFTCTAAHPELKTPLTANITK |
| | | SGNTFRPEVHLLPPPSEELALNELVTLTCLARGFSPKDVLVRWLQGSQELPR |
| | | EKYLTWASRQEPSQGTTTYAVTSILRVAAEDWKKGETFSCMVGHEALPLAFT |
| | | QKTIDRMAGKPTHINVSVVMAEADGTCY (SEQ ID NO: 279) |
| Human | P01880 | APTKAPDVFPIISGCRHPKDNSPVVLACLITGYHPTSVTVTWYMGTQSQPQR |
| IgD | | TFPEIQRRDSYYMTSSQLSTPLQQWRQGEYKCVVQHTASKSKKEIFRWPESP |
| | | KAQASSVPTAQPQAEGSLAKATTAPATTRNTGRGGEEKKKEKEKEEQEERET |
| | | KTPECPSHTQPLGVYLLTPAVQDLWLRDKATFTCFVVGSDLKDAHLTWEVAG |
| | | KVPTGGVEEGLLERHSNGSQSQHSRLTLPRSLWNAGTSVTCTLNHPSLPPQR |
| | | LMALREPAAQAPVKLSLNLLASSDPPEAASWLLCEVSGESPPNILLMWLEDQ |
| | | REVNTSGFAPARPPPQPRSTTFWAWSVLRVPAPPSPQPATYTCVVSHEDSRT |
| | | LLNASRSLEVSYVTDHGPMK (SEQ ID NO: 280) |
| Human | P01871 | GSASAPTLFPLVSCENSPSDTSSVAVGCLAQDELPDSITESWKYKNNSDISS |
| IgM | | TRGFPSVLRGGKYAATSQVLLPSKDVMQGTDEHVVCKVQHPNGNKEKNVPLP |
| | | VIAELPPKVSVFVPPRDGFFGNPRKSKLICQATGESPRQIQVSWLREGKQVG |
| | | SGVTTDQVQAEAKESGPTTYKVTSTLTIKESDWLGQSMFTCRVDHRGLTFQQ |
| | | NASSMCVPDQDTAIRVFAIPPSFASIFLTKSTKLTCLVTDLTTYDSVTISWT |
| | | RQNGEAVKTHTNISESHPNATFSAVGEASICEDDWNSGERFTCTVTHTDLPS |
| | | PLKQTISRPKGVALHRPDVYLLPPAREQLNLRESATITCLVTGFSPADVFVQ |
| | | WMQRGQPLSPEKYVTSAPMPEPQAPGRYFAHSILTVSEEEWNTGETYTCVVA |
| | | HEALPNRVTERTVDKSTGKPTLYNVSLVMSDTAGTCY (SEQ ID NO: |
| | | 281) |
| Human | P01854 | ASTQSPSVFPLTRCCKNIPSNATSVTLGCLATGYFPEPVMVTWDTGSLNGTT |
| IgE | | MTLPATTLTLSGHYATISLLTVSGAWAKQMFTCRVAHTPSSTDWVDNKTFSV |
| | | CSRDFTPPTVKILQSSCDGGGHFPPTIQLLCLVSGYTPGTINITWLEDGQVM |
| | | DVDLSTASTTQEGELASTQSELTLSQKHWLSDRTYTCQVTYQGHTFEDSTKK |
| | | CADSNPRGVSAYLSRPSPFDLFIRKSPTITCLVVDLAPSKGTVNLTWSRASG |
| | | KPVNHSTRKEEKQRNGTLTVTSTLPVGTRDWIEGETYQCRVTHPHLPRALMR |
| | | STTKTSGPRAAPEVYAFATPEWPGSRDKRTLACLIQNFMPEDISVQWLHNEV |
| | | QLPDARHSTTQPRKTKGSGFFVFSRLEVTRAEWEQKDEFICRAVHEAASPSQ |
| | | TVQRAVSVNPGK (SEQ ID NO: 282) |
| Mouse | P01868 | AKTTPPSVYPLAPGSAAQTNSMVTLGCLVKGYFPEPVTVTWNSGSLSSGVHT |
| IgG1 | | FPAVLQSDLYTLSSSVTVPSSPRPSETVTCNVAHPASSTKVDKKIVPRDCGC |
| | | KPCICTVPEVSSVFIFPPKPKDVLTITLTPKVTCVVVDISKDDPEVQFSWFV |
| | | DDVEVHTAQTQPREEQFNSTFRSVSELPIMHQDWLNGKEFKCRVNSAAFPAP |
| | | IEKTISKTKGRPKAPQVYTIPPPKEQMAKDKVSLTCMITDFFPEDITVEWQW |
| | | NGQPAENYKNTQPIMNTNGSYFVYSKLNVQKSNWEAGNTFTCSVLHEGLHNH |
| | | HTEKSLSHSPGK (SEQ ID NO: 283) |
| Mouse | P01863 | AKTTAPSVYPLAPVCGDTTGSSVTLGCLVKGYFPEPVTLTWNSGSLSSGVHT |
| IgG2a | | FPAVLQSDLYTLSSSVTVTSSTWPSQSITCNVAHPASSTKVDKKIEPRGPTI |
| | | KPCPPCKCPAPNLLGGPSVFIFPPKIKDVLMISLSPIVTCVVVDVSEDDPDV |
| | | QISWFVNNVEVHTAQTQTHREDYNSTLRVVSALPIQHQDWMSGKEFKCKVNN |
| | | KDLPAPIERTISKPKGSVRAPQVYVLPPPEEEMTKKQVTLTCMVTDEMPEDI |
| | | YVEWTNNGKTELNYKNTEPVLDSDGSYFMYSKLRVEKKNWVERNSYSCSVVH |
| | | EGLHNHHTTKSFSRTPGK (SEQ ID NO: 284) |
| Mouse | P01867 | KTTPPSVYPLAPGCGDTTGSSVTLGCLVKGYFPESVTVTWNSGSLSSSVHTF |
| IgG2b | | PALLQSGLYTMSSSVTVPSSTWPSQTVTCSVAHPASSTTVDKKLEPSGPIST |
| | | INPCPPCKECHKCPAPNLEGGPSVFIFPPNIKDVLMISLTPKVTCVVVDVSE |
| | | DDPDVQISWFVNNVEVHTAQTQTHREDYNSTIRVVSTLPIQHQDWMSGKEFK |
| | | CKVNNKDLPSPIERTISKIKGLVRAPQVYILPPPAEQLSRKDVSLTCLVVGF |
| | | NPGDISVEWTSNGHTEENYKDTAPVLDSDGSYFIYSKLNMKTSKWEKTDSFS |
| | | CNVRHEGLKNYYLKKTISRSPGK (SEQ ID NO: 285) |
| Mouse | P01864 | AKTTAPSVYPLVPVCGGTTGSSVTLGCLVKGYFPEPVTLTWNSGSLSSGVHT |
| IgG2c | | FPALLQSGLYTLSSSVTVTSNTWPSQTITCNVAHPASSTKVDKKIEPRVPIT |
| | | QNPCPPHQRVPPCAAPDLLGGPSVFIFPPKIKDVLMISLSPMVTCVVVDVSE |
| | | DDPDVQISWFVNNVEVHTAQTQTHREDYNSTLRVVSALPIQHQDWMSGKEFK |
| | | CKVNNRALPSPIEKTISKPRGPVRAPQVYVLPPPAEEMTKKEFSLTCMITGE |
| | | LPAEIAVDWTSNGRTEQNYKNTATVLDSDGSYFMYSKLRVQKSTWERGSLFA |
| | | CSVVHEVLHNHLTTKTISRSLGK (SEQ ID NO: 286) |
| Mouse | P01878 | ESARNPTIYPLTLPPALSSDPVIIGCLIHDYFPSGTMNVTWGKSGKDITTVN |
| IgA | | FPPALASGGRYTMSNQLTLPAVECPEGESVKCSVQHDSNPVQELDVNCSGPT |
| | | PPPPITIPSCQPSLSLQRPALEDLLLGSDASITCTLNGLRNPEGAVFTWEPS |
| | | TGKDAVQKKAVQNSCGCYSVSSVLPGCAERWNSGASFKCTVTHPESGTLTGT |
| | | IAKVTVNTFPPQVHLLPPPSEELALNELLSLTCLVRAFNPKEVLVRWLHGNE |
| | | ELSPESYLVFEPLKEPGEGATTYLVTSVLRVSAETWKQGDQYSCMVGHEALP |
| | | MNFTQKTIDRLSGKPTNVSVSVIMSEGDGICY (SEQ ID NO: 287) |
| Mouse | P01872 | SQSFPNVFPLVSCESPLSDKNLVAMGCLARDFLPSTISFTWNYQNNTEVIQG |
| IgM | | IRTFPTLRTGGKYLATSQVLLSPKSILEGSDEYLVCKIHYGGKNRDLHVPIP |
| | | AVAEMNPNVNVFVPPRDGFSGPAPRKSKLICEATNFTPKPITVSWLKDGKLV |
| | | ESGFTTDPVTIENKGSTPQTYKVISTLTISEIDWLNLNVYTCRVDHRGLTFL |
| | | KNVSSTCAASPSTDILTFTIPPSFADIFLSKSANLTCLVSNLATYETLNISW |
| | | ASQSGEPLETKIKIMESHPNGTFSAKGVASVCVEDWNNRKEFVCTVTHRDLP |
| | | SPQKKFISKPNEVHKHPPAVYLLPPAREQLNLRESATVTCLVKGFSPADISV |
| | | QWLQRGQLLPQEKYVTSAPMPEPGAPGFYFTHSILTVTEEEWNSGETYTCVV |
| | | GHEALPHLVTERTVDKSTGKPTLYNVSLIMSDTGGTCY (SEQ ID NO: |
| | | 288) |
| Mouse | P01881 | DKKEPDMFLLSECKAPEENEKINLGCLVIGSQPLKISWEPKKSSIVEHVFPS |
| IgD | | EMRNGNYTMVLQVTVLASELNLNHTCTINKPKRKEKPFKFPESWDSQSSKRV |
| | | TPTLQAKNHSTEATKAITTKKDIEGAMAPSNLTVNILTTSTHPEMSSWLLCE |
| | | VSGFFPENIHLMWLGVHSKMKSTNFVTANPTAQPGGTFQTWSVLRLPVALSS |
| | | SLDTYTCVVEHEASKTKLNASKSLAISGCYHLLPESDGPSRRPDGPALA |
| | | (SEQ ID NO: 289) |
| Mouse | P06336 | SIRNPQLYPLKPCKGTASMTLGCLVKDYFPNPVTVTWYSDSLNMSTVNFPAL |
| IgE | | GSELKVTTSQVTSWGKSAKNFTCHVTHPPSFNESRTILVRPVNITEPTLELL |
| | | HSSCDPNAFHSTIQLYCFIYGHILNDVSVSWLMDDREITDTLAQTVLIKEEG |
| | | KLASTCSKLNITEQQWMSESTFTCKVTSQGVDYLAHTRRCPDHEPRGVITYL |
| | | IPPSPLDLYQNGAPKLTCLVVDLESEKNVNVTWNQEKKTSVSASQWYTKHHN |
| | | NATTSITSILPVVAKDWIEGYGYQCIVDHPDFPKPIVRSITKTPGQRSAPEV |
| | | YVFPPPEEESEDKRTLTCLIQNFFPEDISVQWLGDGKLISNSQHSTTTPLKS |
| | | NGSNQGFFIFSRLEVAKTLWTQRKQFTCQVIHEALQKPRKLEKTISTSLGNT |
| | | SLRPS (SEQ ID NO: 290) |

TABLE 4
Sequences of antibody light chain constant regions by isotype.

| Light | | |
|---|---|---|
| Chain | Accession | |
| Isotype | No. | Sequence |
| Human Ig | P01834 | RTVAAPSVFIFPPSDEQLKSGTASVVCLLNNFYPREAKVQWKVD |
| kappa | | NALQSGNSQESVTEQDSKDSTYSLSSTLTLSKADYEKHKVYACE |
| | | VTHQGLSSPVTKSENRGEC (SEQ ID NO: 291) |
| Human Ig | P0CG04 | GQPKANPTVTLFPPSSEELQANKATLVCLISDFYPGAVTVAWKA |
| lambda 1 | | DGSPVKAGVETTKPSKQSNNKYAASSYLSLTPEQWKSHRSYSCQ |
| | | VTHEGSTVEKTVAPTECS (SEQ ID NO: 292) |
| Human Ig | P0DOY2 | GQPKAAPSVTLFPPSSEELQANKATLVCLISDFYPGAVTVAWKA |
| lambda 2 | | DSSPVKAGVETTTPSKQSNNKYAASSYLSLTPEQWKSHRSYSCQ |
| | | VTHEGSTVEKTVAPTECS (SEQ ID NO: 293) |
| Human Ig | P0DOY3 | GQPKAAPSVTLFPPSSEELQANKATLVCLISDFYPGAVTVAWKA |
| lambda 3 | | DSSPVKAGVETTTPSKQSNNKYAASSYLSLTPEQWKSHKSYSCQ |
| | | VTHEGSTVEKTVAPTECS (SEQ ID NO: 294) |
| Human Ig | P0CF74 | GQPKAAPSVTLFPPSSEELQANKATLVCLISDFYPGAVKVAWKA |
| lambda 6 | | DGSPVNTGVETTTPSKQSNNKYAASSYLSLTPEQWKSHRSYSCQ |
| | | VTHEGSTVEKTVAPAECS (SEQ ID NO: 295) |
| Human Ig | A0M8Q6 | GQPKAAPSVTLFPPSSEELQANKATLVCLVSDENPGAVTVAWKA |
| lambda 7 | | DGSPVKVGVETTKPSKQSNNKYAASSYLSLTPEQWKSHRSYSCR |
| | | VTHEGSTVEKTVAPAECS (SEQ ID NO: 296) |
| Mouse Ig | P01837 | RADAAPTVSIFPPSSEQLTSGGASVVCFLNNFYPKDINVKWKID |
| kappa | | GSERQNGVLNSWTDQDSKDSTYSMSSTLTLTKDEYERHNSYTCE |
| | | ATHKTSTSPIVKSENRNEC (SEQ ID NO: 297) |
| Mouse Ig | A0A0G2JE99 | XQPKSSPSVTLFPPSSEELETNKATLVCTITDFYPGVVTVDWKV |
| lambda 1 | | DGTPVTQGMETTQPSKQSNNKYMASSYLTLTARAWERHSSYSCQ |
| | | VTHEGHTVEKSLSRADCS (SEQ ID NO: 298) |
| Mouse Ig | Q99JC1 | GQPKSTPTLTVFPPSSEELKENKATLVCLISNFSPSGVTVAWKA |
| lambda 2 | | NGTPITQGVDTSNPTKEGNKFMASSFLHLTSDQWRSHNSFTCQV |
| | | THEGDTVEKSLSPAECL (SEQ ID NO: 299) |
| Mouse Ig | P01845 | QPKSTPTLTMFPPSPEELQENKATLVCLISNFSPSGVTVAWKAN |
| lambda 3 | | GTPITQGVDTSNPTKEDNKYMASSFLHLTSDQWRSHNSFTCQVT |
| | | HEGDTVEKSLSPAECL (SEQ ID NO: 300) |
| Mouse Ig | A0A0G2JEK5 | XQPKATPSVNLFPPSSEELKTKKATLVCMITEFYAAAVRVAWKA |
| lambda 4 | | DGTPFTQGVETTQPPKQRDNMASSYLLFTAEAWESHSSYSCHVT |
| | | HEGNTVEKSLSRAECS (SEQ ID NO: 301) |

TABLE 5
Nucleotide sequences of antibody complementary determining regions,
variable heavy chain domains, and variable light chain domains.

| | Complementarity | | |
|---|---|---|---|
| Antibody | Determining Region | Variable Heavy | Variable Light |
| Clone | (CDR) sequences | Chain (VH) Sequence | Chain (VL) Sequence |
| 1E2 | ggattcaccttcagtgac | caggtgcagctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctcgg | accatcaactgcaagtcca |
| | gccatt - SEQ ID | gattcaccttcagtgacta | gccagaatattttgtacag |
| | NO: 303 | ctacatgaactggatccgc | ttccaacaataagaactac |
| | gcgagagagcgtagagat | ctgcctccagggaagggcc | ttagcttggtaccagcaga |
| | ggccacaaaatctttgac | tggagtgggtttcatacat | aagcaggacagcctcctaa |
| | tgt - SEQ ID NO: | tgatagtagtgctggtgcc | gttgctcattttctggtca |
| | 304 | atttactatgcagactctg | tctacccgggaatccgggg |
| | cagaatattttgtacagt | tgaggggccgattcaccgt | tccctgaccgattcagtgg |
| | tccaacaataagaactac - | ctccagggacgacgccaag | cagcgggtctgggacagat |
| | SEQ ID NO: 305 | aattcactatttctgcaca | ttcactctcaccatcaaca |
| | tggtcatct - SEQ ID | tgaacagcctgagagccga | gcctgcaggctgaagatgt |
| | NO: 306 | tgacacggccgtttattac | ggcagtttattactgtcag |
| | cagcaatattacagtagt | tgtgcgagagagcgtagag | caatattacagtagtccgt |
| | ccgtataat - SEQ ID | atggccacaaaatctttga | ataattttggccgggggac |
| | NO: 307 | ctgttggggcccgggaacc | cacactggagatcaaa - |
| | | ctggtcaccgtctcttca - | SEQ ID NO: 309 |
| | | SEQ ID NO: 308 | |
| 1H10 | ggattcaccttcagtgac | caggtgcacctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctctg | accatcaactgcaagtcca |
| | gccatt - SEQ ID | gattcaccttcagtgacta | gccagaatattttatacag |
| | NO: 303 | ctacatgaactggatccgt | ttccaacaataagaactat |
| | gcgagagagcgtagagat | atgcctccagggaagggcc | ttagcttggtaccagcaga |
| | ggccacaaaatctttgac | tggagtggatttcatacat | aagcaggacagcctcctaa |
| | tgt - SEQ ID NO: | tgatagtagtgctggtgcc | gttgctcattttctggtca |
| | 304 | atttactatgcagactctg | tctacccgggaatccgggg |
| | cagaatattttatacagt | tgaagggccgattcaccgt | tccctgaccgattcagtgg |
| | tccaacaataagaactat - | ctccagggacgacgccaag | cagcgggtctgggacagat |
| | SEQ ID NO: 310 | aactcactgtttctacaca | ttcactctcaccatcagca |
| | tggtcatct - SEQ ID | tgaacaacctgagagccga | gcctgcaggctgaagatgt |
| | NO: 306 | tgacacggccgtttattac | ggcagtttattactgtcag |
| | cagcaatattacagtagt | tgtgcgagagagcgtagag | caatattacagtagtccgt |
| | ccgtataat - SEQ ID | atggccacaaaatctttga | ataattttggccgggggac |
| | NO: 307 | ctgttggggcccgggaacc | cacactggagatcaaa - |
| | | ctggtcaccgtctcctca - | SEQ ID NO: 312 |
| | | SEQ ID NO: 311 | |
| 2A10 | ggatacagttttacatac | gaggtgcagctggtgcagt | gacatccagatgacccagt |
| | tactgg - SEQ ID | ctggagcagaggtgaaaaa | ctccatcctccctgtctgc |
| | NO: 313 | gcccggggagtctctgaag | atctgtcggagacagactc |
| | atctatcctggtgactct | atctcctgtaagggttctg | accatcacttgccgggcaa |
| | gatacc - SEQ ID | gatacagttttacatacta | gtcagaacattaacacctt |
| | NO: 314 | ctggatcggctgggtgcgc | tttaaattggtatcaacag |
| | actacacatgcgcgcaac | cagatgcccgggaaaggcc | aacccagggaaagccccta |
| | tggaacaacgtggcctac - | tggagtggatggggatcat | aggtcctgatctatgctgc |
| | SEQ ID NO: 315 | ctatcctggtgactctgat | atccagtttggaaagtggg |
| | cagaacattaacaccttt - | accagatacagcccgtcct | gtcccatcaaggttcagtg |
| | SEQ ID NO: 316 | tccaaggccaggtcaccat | gcagtggatctgggacaga |
| | gctgcatcc - SEQ ID | ctcagccgacaagtccatc | tttcactctcaccatcagc |
| | NO: 317 | agtaccgcctatttgcagt | agtctgcaacctgaagatt |
| | caacagagttctagtacc | ggagcagcctgaaggcctc | ttgcaacttactactgtca |
| | ccgtggacg - SEQ ID | ggacaccgccatgtattac | acagagttctagtaccccg |
| | NO: 318 | tgtactacacatgcgcgca | tggacgttcggccaaggga |
| | | actggaacaacgtggccta | ccaaggtggaaatcaaac |
| | | ctggggccagggaaccctg | - SEQ ID NO: 320 |
| | | gtcaccgtctcctca - | |
| | | SEQ ID NO: 319 | |
| 2B9 | ggattcacctttagtggc | gaggtgcagcttttggagt | gacatccagatgacccagt |
| | ttcgcc - SEQ ID | cggggggaggcttggcaca | ctccatcctccctgtctgc |
| | NO: 321 | gccggggaagtccctgaga | atctgtaggagacagagtc |
| | attagtgggagtggtgat | ctctcctgtgcagcctctg | accatcacttgccgggcaa |
| | atcaca - SEQ ID | gattcacctttagtggctt | gtcagagcattagcagcta |
| | NO: 322 | cgccatgagctgggtccgc | tttaaattggtatcagcag |
| | gcgaaagacccatataac | caggctcctgggaaggggc | aaaccagggaaagccccta |
| | tggaaccacggggtctac | tggagtgggtctcaggtat | aattcctgatctatggtgc |
| | ggcatggacgtc - SEQ | tagtgggagtggtgatatc | atccagtttgcaaagtggg |
| | ID NO: 323 | acatactatgcagactccg | gtcccatcaaggttcagtg |
| | cagagcattagcagctat - | tgaagggccggttcaccat | gcagtggatctgggactga |
| | SEQ ID NO: 3244 | ttccagagacaattccaag | tttcactctcaccatcagc |
| | ggtgcatcc - SEQ ID | agtacgctgtatcttcaaa | agtctgcaacctgaagatt |
| | NO: 325 | tgaacagcctgggagccga | tcgcagcttactactgtca |
| | caacagagtttcaatgcc | ggacacggccgtgtattac | acagagtttcaatgcccct |
| | cctctcact - SEQ ID | tgtgcgaaagacccatata | ctcactttcggcggcggga |
| | NO: 326 | actggaaccacggggtcta | ccaaggtggagatcaaa - |
| | | cggcatggacgtctggggc | SEQ ID NO: 328 |
| | | caagggaccacggtcaccg | |
| | | tctcctca - SEQ ID | |
| | | NO: 327 | |
| 2F7 | ggattcaccttcagtgac | caggtgcagctggtggagt | gacatcgtgatgacccagt |
| | tactac - SEQ ID | ctgggggaggcttggtcaa | ctccagactccctggctgt |
| | NO: 302 | gcctggagggtccctgaga | gtctctgggcgagagggcc |
| | attgatagtagtgctggt | ctctcctgcgtagcctctg | accatcaactgcaagtcca |
| | gccatt - SEQ ID | gattcaccttcagtgacta | gccagaatattttatacac |
| | NO: 303 | ctacatgaactggatccgc | ttccaacaataagaactac |
| | gcgagagaacgtagagat | ctgcctccagggagggggc | ttaggttggtaccagcaga |
| | ggccacaaaatctttgac | tgcagtggatttcatacat | aagcaggacagcctcctaa |
| | tgg - SEQ ID NO: | tgatagtagtgctggtgcc | actgctcatttactggtca |
| | 329 | atttactacgcagactctg | tctacccggaattccgggg |
| | cagaatattttatacact | tgaagggccgattcaccgt | tccctgaccgcttcagtgg |
| | tccaacaataagaactac - | ctccagggacgacgccaag | cagcgggtctgggacagat |
| | SEQ ID NO: 330 | aattcactttatctgcaaa | ttcactctcaccatcagca |
| | tggtcatct - SEQ ID | tgaacagcctgagagccga | gcctgcaggctgaggatgt |
| | NO: 306 | tgacacggccgtttattac | ggcagtttattactgtcat |
| | catcagtattacagtagt | tgtgcgagagaacgtagag | cagtattacagtagtccgt |
| | ccgtacact - SEQ ID | atggccacaaaatctttga | acacttttggccaggggac |
| | NO: 331 | ctggtggggccagggaacc | caacctggagatcaaa - |
| | | ctggtcaccgtctcctca - | SEQ ID NO: 3333 |
| | | SEQ ID NO: 332 | |
| 2F11 | ggatacagttttaccaac | gaggtgcagctggtgcagt | gacatccagatgacccagt |
| | tactgg - SEQ ID | ctggagcagaggtgaaaaa | ctccatcctccctgtctgc |
| | NO: 334 | gcccggggagtctctgaag | atctgtaggagacagagtc |
| | atctatcctggtgactct | atctcctgtaagggttctg | accatcacttgccgggcaa |
| | gatacc - SEQ ID | gatacagttttaccaacta | gtcagagcgtcaacaactt |
| | NO: 314 | ctggatcggctgggtgcgc | tttaaattggtatcagcag |
| | gcgacacatgcgcgcagc | cagatgcccgggaaaggcc | aaaccagggacagccccta |
| | tggaactacgtggcctac - | tggagtggatggggatcat | aactcctgatctatgctgc |
| | SEQ ID NO: 335 | ctatcctggtgactctgat | atccagtttgcaaggtggg |
| | cagagcgtcaacaacttt - | accagatacagcccgtcct | gtcccatcaaggttcagtg |
| | SEQ ID NO: 336 | tccaaggccaggtcaccct | gcagtggctctgggacaga |
| | gctgcatcc - SEQ ID | ctcagccgacaagtccatc | tttcactctcaccatcagc |
| | NO: 317 | agtaccgcctacctacagt | agtctgcaacctgaagatt |
| | caacagagtaacactacc | ggaacagcctgaaggcctc | ttgcaacttactactgtca |
| | ccgtggacg - SEQ ID | ggacaccgccatgtattac | acagagtaacactaccccg |
| | NO: 337 | tgtgcgacacatgcgcgca | tggacgttcggccaaggga |
| | | gctggaactacgtggccta | ccaaggtggaaatcaaa - |
| | | ctggggccagggaaccctg | SEQ ID NO: 339 |
| | | gtcaccgtcgcctcat | |
| | | SEQ ID NO: 338 | |
| 2H1 | ggattcaccttcagtgac | caggtgcagctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctctg | accatcaactgcaagtcca |
| | gccatt - SEQ ID | gattcaccttcagtgacta | gccagaatattttatacag |
| | NO: 303 | ctacatgaactggatccgc | ttccaacaataagaactac |
| | gcgagagagcgtagagat | ctgcctccagggaagggcc | ttagcttggtaccagcaga |
| | ggccacaaaatctttgac | tggagtggatttcctacat | aaacaggacagcctcctaa |
| | tgt - SEQ ID NO: | tgatagtagtgctggtgcc | gttgctcattttctggtca |
| | 304 | atttactatgcagactctg | tctacccgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aactcactatttctgcaca | ttcactctcaccatcagca |
| | tggtcatct - SEQ ID | tgaacagcctgagagccga | gcctgcaggctgaggatgt |
| | NO: 306 | tgacacggccgtttattac | ggcagtttattactgtcag |
| | cagcaatattacagtagt | tgtgcgagagagcgtagag | caatattacagtagtccgt |
| | ccgtataat - SEQ ID | atggccacaaaatctttga | ataattttggccgggggac |
| | NO: 307 | ctgttggggcccgggaacc | cacactggagatcaaa - |
| | | ctggtcaccgtctcctca - | SEQ ID NO: 342 |
| | | SEQ ID NO: 341 | |
| 4E6 | ggattcacctttgatgat | gaagtgcagttggtggagt | gacatcgtgatgacccagt |
| | tatgcc - SEQ ID | ctgggggagacttggtaca | ctccagactccctggctgt |
| | NO: 343 | gcctggcaggtccctgaga | gtctctgggcgagagggcc |
| | attagttggagtggtgtt | ctctcctgtgcagcctctg | accatcaactgcaagtcca |
| | accatg - SEQ ID | gattcacctttgatgatta | gccagagtgttttgtacaa |
| | NO: 344 | tgccatgcactgggtccgg | gtccaacaataagaactac |
| | gcaagaggggatggaacc | caaggtccagggaagggcc | ttggattggtatcagcaga |
| | aatgcttttgatatc - | tggagtgggtctcaggcat | aaccaggacagcctcctaa |
| | SEQ ID NO: 345 | tagttggagtggtgttacc | gctgctcatttattgggca |
| | cagagtgttttgtacaag | atgggctatgcggactctg | tcttcccgggaatccgggg |
| | tccaacaataagaactac - | tgaagggccgattcaccat | tccctgaccgattcagtgg |
| | SEQ ID NO: 346 | ttccagagacaacgccaag | cagcgggtctgggacagat |
| | tgggcatct - SEQ ID | aactccctgtacctgcgaa | ttcactctgaccatcagca |
| | NO: 347 | tgaacagtctgagagctga | gcctgcaggctgaagatgt |
| | cagcaatattatagtgtt | cgacacggccttctattac | ggcagtttattactgtcag |
| | ccgctcact - SEQ ID | tgtgcaagaggggatggaa | caatattatagtgttccgc |
| | NO: 348 | ccaatgcttttgatatctg | tcactttcggcggagggac |
| | | gggccacgggacaatggtc | caaggtggagatcaaa |
| | | accgtctcttca - SEQ | SEQ ID NO: 350 |
| | | ID NO: 349 | |
| 4F7 | ggattcaccttcagtgac | caggtgcagctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctctg | accatcaactgcaagtcca |
| | gccatt - SEQ ID | gattcaccttcagtgacta | gccagaatattttatacag |
| | NO: 303 | ctacatgaactggatccgc | ttccaacaataagaactac |
| | gcgagagagcgtagagat | ctgcctccagggaagggcc | ttagcttggtaccagcaga |
| | ggccacaaaatctttgac | tggagtggatttcatacat | aagcaggacagcctcctaa |
| | tgt - SEQ ID NO: | tgatagtagtgctggtgcc | gttgctcattttctggtca |
| | 304 | atttactatgcagactctg | tctacccgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aactcactatttctacaca | ttcactctcaccatcagca |
| | tggtcatct - SEQ ID | tgaacaacctgagagccga | gcctgcaggctgaagatgt |
| | NO: 306 | tgacacggccgtttattac | ggcagtttattactgtcag |
| | cagcaatattacagtagt | tgtgcgagagagcgtagag | caatattacagtagtccgt |
| | ccgtataat - SEQ ID | atggccacaaaatctttga | ataattttggccgggggac |
| | NO: 307 | ctgttggggcccgggaacc | cacactggagatcaaa - |
| | | ctggtcaccgtctcctca - | SEQ ID NO: 352 |
| | | SEQ ID NO: 351 | |
| 5A8 | ggattcaccttcagtgac | caggtgcagctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctctg | accatcaactgcaagtcca |
| | gccatt - SEQ ID | gattcaccttcagtgacta | gccagaatattttatacag |
| | NO: 303 | ctacatgaactggatccgc | ttccaacaataagaactac |
| | gcgagagagcgtagagat | ctgcctccagggaggggcc | ttagcttggtaccagcaga |
| | ggccacaaaatctttgac | tggagtggatttcatacat | aagcaggacagcctcctaa |
| | tat - SEQ ID NO: | tgatagtagtgctggtgcc | gttgctcattttctggtca |
| | 353 | atttactatgcagactctg | tctacccgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aagtcactatttctgtaca | ttcactctcaccatcagca |
| | tggtcatct - SEQ ID | tgaacagcctgagagccga | gcctgcaggctgaagatgt |
| | NO: 306 | tgacacggccgtttattac | ggcagtttattactgtcac |
| | caccaatattacagtagt | tgtgcgagagagcgtagag | caatattacagtagtccgt |
| | ccgtataat - SEQ ID | atggccacaaaatctttga | ataattttggccgggggac |
| | NO: 354 | ctattggggcccgggaacc | cacactggagatcaaa - |
| | | ctggtcaccgtctcctca - | SEQ ID NO: 356 |
| | | SEQ ID NO: 355 | |
| 6E1 | ggattcacctttagcagc | gaggtgcagttgttggagt | gacatccagatgacccagt |
| | tttgcc - SEQ ID | ctgggggaggcttggtaca | ctccgtcctccctgtctgc |
| | NO: 357 | gcctggggggtccctgaga | atctgtaggaggcagagto |
| | attagtggtagtggtggt | ctctcctgtgcagcctctg | accatcacttgccggacaa |
| | agcaca - SEQ ID | gattcacctttagcagctt | gtcagagcattagcggcta |
| | NO: 358 | tgccatgagctgggtccgc | tttaaattggtatcagcag |
| | gcgaaagataaaaggaac | caggctccagggaaggggc | aaagcagggaaagccccta |
| | tggaactacgggattgat | tggagtgggtctcaggtat | aactcctgatctatgctac |
| | tcttttgatttc - SEQ | tagtggtagtggtggtagc | atccaatttgcaaagtggg |
| | ID NO: 359 | acatactacgcagactccg | gtcccatcaaggttcactg |
| | cagagcattagcggctat - | tgaagggccggttcaccat | gcagtggctctgggacaga |
| | SEQ ID NO: 360 | ctccagagacaattccaag | tttcactctcaccatcagc |
| | gctacatcc - SEQ ID | aacacgctgtatctgcaaa | agtctgcaacctgaagatt |
| | NO: 361 | tgaacagcctgagagccga | ttgcagcttactactgtca |
| | catcagagtcacagtccc | ggacacggccatatattac | tcagagtcacagtccccca |
| | ccattcact - SEQ ID | tgtgcgaaagataaaagga | ttcactttcggccctggga |
| | NO: 362 | actggaactacgggattga | ccaaagtggctttcaaa - |
| | | ttcttttgatttctggggc | SEQ ID NO: 364 |
| | | caagggacgatggtcaccg | |
| | | tctcttca - SEQ ID | |
| | | NO: 363 | |
| 6H12 | ggattcaccttcagtagt | gaggtgcagttggtggagt | gacatccagttgacccagt |
| | tatgaa - SEQ ID | ctgggggaggcttggtcca | ctccatccttcctgtctgc |
| | NO: 365 | gcctggagggtccctgaga | atctgtaggagacagagtc |
| | attagtagtggtggtagt | ctctcctgtgcagcctctg | accatcacttgctgggcca |
| | tctata - SEQ ID | gattcaccttcagtagtta | gtcaggacattagcagtta |
| | NO: 366 | tgaaatgaactgggtccgc | tttagcctggtatcagcaa |
| | gcgagagtgggtggaagc | caggctccagggaagggac | aaaccagggaaagccccta |
| | tactactactcctacgct | tggagtgggtttcatacat | agctcctgatctatgctgc |
| | atggacgtc - SEQ ID | tagtagtggtggtagttct | atccactttgcaaagtggg |
| | NO: 367 | ataaaatacgcagactctg | gtcccatcaaggttcaccg |
| | caggacattagcagttat - | tgaggggccgattcgcctt | gcagtggatctgggacaga |
| | SEQ ID NO: 368 | ctccagagacaacgccgag | attcactctcacaatcagc |
| | gctgcatcc - SEQ ID | aactcagtgcatctgcaaa | agcctgcagcctgaagatt |
| | NO: 317 | tgaacagcctgagagccga | ttgcaacttattactgtca |
| | caacagcctcataattcc | ggacacggctgtttattac | acagcctcataattcccca |
| | ccattcagt - SEQ ID | tgtgcgagagtgggtggaa | ttcagtttcggccctggga |
| | NO: 369 | gctactactactcctacgc | ccaaagtggatgtcaaa - |
| | | tatggacgtctggggccaa | SEQ ID NO: 371 |
| | | gggaccgcggtcaccgtct | |
| | | cctca - SEQ ID NO: | |
| | | 370 | |
| 7A4 | ggattcaccttcagtgac | caggtgcagctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagtgctggt | ctctcctgtgtagcctctg | accatcaactgcaagtcca |
| | gccatt - SEQ ID | gattcaccttcagtgacta | gccagaatattttatacag |
| | NO: 303 | ctacatgaactggatccgc | ttccaacaataagaactac |
| | gtgagagagcgtagagat | ctgcctccagggaagggcc | ttagcttggtaccagcaga |
| | ggccacaaaatctttgac | tggagtggatttcatacat | aagcaggacagcctcctaa |
| | tat - SEQ ID NO: | tgatagtagtgctggtgcc | gttgctcattttctggtca |
| | 372 | atttactatgcagactctg | tctattcgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aactcactatttctgcaca | ttcactctcaccatcagca |
| | tggtcatct - SEQ ID | tgaacagcctgagagccga | gcctgcaggctgaagatgt |
| | NO: 306 | tgacacggccgtttattac | ggcagtttattattgtcag |
| | cagcaatattacagtagt | tgtgtgagagagcgtagag | caatattacagtagtccgt |
| | ccgtataat - SEQ ID | atggccacaaaatctttga | ataattttggccgggggac |
| | NO: 307 | ctattggggcccgggaacc | cacactggagatcaaa - |
| | | ctggtcaccgtctcctcc - | SEQ ID NO: 374 |
| | | SEQ ID NO: 373 | |
| 7E5 | ggattcacctttagtggc | gaggtgcagctgttggaat | gacatccagatgacccagt |
| | ttcgcc - SEQ ID | ctgggggaggcctgataca | ctccatcctccctgtctgc |
| | NO: 321 | accgggggggtccctgaga | atccgtaggagacagagtc |
| | attagtgggagtggtgat | ctctcctgtgcagcctctg | accatcacttgccgggcaa |
| | atcaca - SEQ ID | gattcacctttagtggctt | gtcagagcattagcaccta |
| | NO: 322 | cgccatgagctgggtccgc | tttaaattggtatcagcag |
| | gggaaagatccatataac | caggctcctgggaaggggc | aagccagggaaagccccta |
| | tggaatcacggggtctac | tggagtgggtctcaggtat | agttcctgatctatggtgc |
| | ggcatggacgtc - SEQ | tagtgggagtggtgatatc | ttccagtttgcaaagtggg |
| | ID NO: 375 | acatactatggagactccg | gtcccatcaaggttcagtg |
| | cagagcattagcacctat - | tgaagggccggttcaccat | gcagtggatctgggacaga |
| | SEQ ID NO: 376 | ttccagagacaattccaag | tttctctctcaccatcagc |
| | ggtgcttcc - SEQ ID | agtacgctgtatctgcaaa | agtctgcaccctggagact |
| | NO: 377 | tgaacagcctgagagacgc | tcgcaacttactactgtca |
| | caacagagttacaatatc | ggacacggccgtatattat | acagagttacaatatccct |
| | cctctcact - SEQ ID | tgtgggaaagatccatata | ctcactttcggcggaggga |
| | NO: 378 | actggaatcacggggtcta | ccaaggtggagatcaaa - |
| | | cggcatggacgtctggggc | SEQ ID NO: 380 |
| | | caagggaccacggtcaccg | |
| | | tctcctca - SEQ ID | |
| | | NO: 379 | |
| 7H8 | ggattcgcctttcacacc | gaggtgcagttgttggagt | gacatccagatgacccagt |
| | tatgac - SEQ ID | ctgggggaggcttggtaca | ctccttccaccctgtctgc |
| | NO: 381 | gcctggggggtccctgaga | atctgtaggagacagagtc |
| | gttgttggtagtggtatt | ctctcctgtgcagcctctg | accatcacttgccgggcca |
| | aacaca - SEQ ID | gattcgcctttcacaccta | gtcagagcataagtaactg |
| | NO: 382 | tgacatgacctgggtccgc | gttggcctggtatcagcag |
| | gcgaaagatagcagcagt | caggctccagggaagggac | aaaccagggaaagccccta |
| | tggttttccctccactac - | tggagtgggtctcaggtgt | aactcctgatctataaggc |
| | SEQ ID NO: 383 | tgttggtagtggtattaac | gtctagtttagaaagtggg |
| | cagagcataagtaactgg - | acatactacgcagactccg | gtcccatcaaggttcagcg |
| | SEQ ID NO: 384 | tgaagggccggttcaccat | gcagtggatctgggacaga |
| | aaggcgtct - SEQ ID | ttccagagacaattccaag | tttcactctcaccatcagc |
| | NO: 385 | agcacgctctatctgcaaa | agcctgcagcctgatgatt |
| | caacagtatagaagttat | tgaacagtctgagagccga | ttgcaagttattactgcca |
| | gcgtacact - SEQ ID | ggacacggccgtatattac | acagtatagaagttatgcg |
| | NO: 386 | tgtgcgaaagatagcagca | tacacttttggccagggga |
| | | gttggttttccctccacta | ccaagctggagatcaca - |
| | | ctggggccagggaaccctg | SEQ ID NO: 388 |
| | | gtcaccgtctcctca - | |
| | | SEQ ID NO: 387 | |
| 8A11 | ggattcaccttcagtgac | caggtgcagttggtggagt | gacatcgtgatgacccagt |
| | tattac - SEQ ID | ctgggggagacttggtcaa | ctccagactccctggctgt |
| | NO: 389 | gcctggagggtccctgaga | gtctctgggcgagagggcc |
| | attgatagtagtgctggt | ctctcctgtgtagcctctg | accatcaactgcaagtcca |
| | gccact - SEQ ID | gattcaccttcagtgacta | gccagaatattttatacag |
| | NO: 390 | ttacatgaactggatccgc | ttccaacaataagaactac |
| | gcgagagagcgtagaagt | ctgcctccagggaagggcc | ttagcttggtaccagcaga |
| | ggccacaaaatctttgac | tggagtggatttcatacat | aagcaggacagcctcctaa |
| | tgt - SEQ ID NO: | tgatagtagtgctggtgcc | gttgctcattttctggtca |
| | 391 | acttactatgcagactctg | tctacccgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aattcactatttctgcaca | ttcactctcaccatcagca |
| | tggtcatct - SEQ ID | tgaacagtctgagagccga | gcctgcaggctgaagatgt |
| | NO: 306 | ggacacggccgtttattac | ggcagtttattactgtcag |
| | cagcaatattacagtagt | tgtgcgagagagcgtagaa | caatattacagtagtccgt |
| | ccgtataat - SEQ ID | gtggccacaaaatctttga | ataattttggccgggggac |
| | NO: 307 | ctgttggggcccgggaacc | cacactggagatcaaa - |
| | | ctggtcaccgtctcctca - | SEQ ID NO: 352 |
| | | SEQ ID NO: 392 | |
| 8D10 | ggattcaccttcagtgac | caggtgcacctggtggagt | gacatcgtgatgacccagt |
| | tactac - SEQ ID | ctgggggagacttggtcaa | ctccagactccctggctgt |
| | NO: 302 | gcctggagggtccctgaga | gtctctgggcgagagggcc |
| | attgatagtagcgctggt | ctctcctgtgtagcctctg | accatcaactgcaagtcca |
| | gccatt - SEQ ID | gattcaccttcagtgacta | gccagaatattttatacag |
| | NO: 393 | ctacatgaactggatccgc | ttccaacaataagaactac |
| | gcgagagagcgtagagat | ctgcctccagggaagggcc | ttagcttggtaccagcaga |
| | ggccacaaaatctttgac | tggagtggatttcatacat | aagcaggacagcctcctaa |
| | tat - SEQ ID NO: | tgatagtagcgctggtgcc | gttgctcattttctggtca |
| | 353 | atttactatgcagactctg | tctacccgggaatccgggg |
| | cagaatattttatacagt | tgaagggccgattcaccgt | tccctgaccgattcagtgg |
| | tccaacaataagaactac - | ctccagggacgacgccaag | cagcgggtctgggacagat |
| | SEQ ID NO: 340 | aactcaatgtttctgcaca | ttcactctcaccatcagca |
| | tggtcatct - SEQ ID | tgaacagcctgagagccga | gcctgcaggctgaagatgt |
| | NO: 306 | tgacacggccgtttattac | ggcagtttattactgtcag |
| | cagcaatattacagtagt | tgtgcgagagagcgtagag | caatattacagtagtccgt |
| | ccgtataat - SEQ ID | atggccacaaaatctttga | ataattttggccgggggac |
| | NO: 307 | ctattggggcccgggaacc | cacactggagatcaaa - |
| | | ctggtcaccgtctcctca - | SEQ ID NO: 352 |
| | | SEQ ID NO: 394 | |
| 9B4 | ggtttcaccttcagtgac | caggtgcaactggtggagt | gacatcgtgatgacccagt |
| | tactac - SEQ ID | ctgggggagacttggtcaa | ctccagactccctggctct |
| | NO: 395 | gcctggagggtccctgaga | gtctctgggcgagagggcc |
| | attgatagtagtgctggt | ctctcctgtgtagcctctg | accattaactgcaagtcca |
| | gccatt - SEQ ID | gtttcaccttcagtgacta | gccagaatattttatacag |
| | NO: 303 | ctacatgaactggatccgc | ttccaacaataagaactac |
| | gcgagagagcggagagaa | ctgcctccagggaagggcc | ttagcttgttaccaacaga |
| | ggccacaaaatctttgac | tggagtggatttcatacat | aagcaggacagcctcctaa |
| | ttt - SEQ ID NO: | tgatagtagtgctggtgcc | gttgctcattttctggtca |
| | 396 | atttactatgcagactctg | tctacccgggaatccgggg |
| | cagaatattttatacagt | tgaaggggcgattcaccgt | tccctgaccgattcagtgg |
| | tccaacaataagaactac - | ctccagggacgacgccaag | cagcgggtctgggacagat |
| | SEQ ID NO: 340 | aactcattatttctgcaca | ttcactctcaccatcagca |
| | tggtcatct - SEQ ID | tgaacagcctgagagccga | gcctgcaggctgaagatgt |
| | NO: 306 | tgacacggccgtttattac | ggcagtttattactgtcag |
| | cagcaatattacagtagt | tgtgcgagagagcggagag | caatattacagtagtccgt |
| | ccgtataat - SEQ ID | aaggccacaaaatctttga | ataatttcggccgggggac |
| | NO: 30 | cttttggggcccgggaacc | cacactggagatcaaa - |
| | | ctggtcaccgtctcctca - | SEQ ID NO: 398 |
| | | SEQ ID NO: 397 | |
| 9G9 | ggcttcacctttcacaac | gaggtgcagctgttggagt | gacatccagatgacccagt |
| | tatgcc - SEQ ID | ctgggggaggcttggtaca | ctccatcctccctgtctgc |
| | NO: 399 | gcctggggggtccctgaga | atctgtaggagacagagtc |
| | attagtggtagtggtggt | ctctcctgtgcagcctctg | accatcacttgccgggcaa |
| | accgca - SEQ ID | gcttcacctttcacaacta | gtcagagcattggcagctt |
| | NO: 400 | tgccatgagctgggtccgc | tttaatttggtatcagcag |
| | gcgaaagaccgatttttg | caggctccagggaaggggc | aaaccagggaaagccccta |
| | gagtgggtagaggggttc | tggagtgggtctcaggtat | agctcctgatttatgctgc |
| | gactcc - SEQ ID | tagtggtagtggtggtacc | atccagtttgcaaagtggg |
| | NO: 401 | gcatactacgcagactccg | gtcccatcaaggttcagtg |
| | cagagcattggcagcttt - | tgaagggccggttcaccat | gcagtggatctgggacaga |
| | SEQ ID NO: 402 | ttccagagacaattccaag | tttcactctcattatcagc |
| | gctgcatcc - SEQ ID | aacacgctgtatctgcaaa | agtctgcaacctgaagatt |
| | NO: 317 | tgaacagcctgagagccga | ttgcaacttattactgtca |
| | caacagagttacaatacc | cgacacggccttatattac | acagagttacaataccccg |
| | ccgctcact - SEQ ID | tgtgcgaaagaccgatttt | ctcactttcggcggaggga |
| | NO: 403 | tggagtgggtagaggggtt | ccaaagtggagatcaaa - |
| | | cgactcctggggccaggga | SEQ ID NO: 405 |
| | | accctggtcaccgtctcct | |
| | | ca - SEQ ID NO: 404 | |
| 9H9 | ggattcaccttcagtagt | gaggtgcagttggtggagt | gacatccagttgacccagt |
| | tatgaa - SEQ ID | ctgggggaggcttggtcca | ctccatccttcctgtctgc |
| | NO: 365 | gcctggagggtccctgaga | atctgtaggagacagagtc |
| | attagtagtggtggtagt | ctctcctgtgcagcctctg | accatcacttgctgggcca |
| | tccata - SEQ ID | gattcaccttcagtagtta | gtcaggacattagcagtta |
| | NO: 406 | tgaaatgaactgggtccgc | tttagcctggtatcagcaa |
| | gcgagagtgggtggaagc | caggctccagggaagggac | aaaccagggaaagccccta |
| | tactactactcctacgct | tggagtgggtttcatacat | agctcctgatctatgctgc |
| | atggacgtc - SEQ ID | tagtagtggtggtagttcc | atccactttgcaaagtggg |
| | NO: 367 | ataaaatacgcagactctg | gtcccatcaaggttcagcg |
| | caggacattagcagttat - | tgaggggccgattcacctt | gcagtggatctgggacaga |
| | SEQ ID NO: 368 | ctccagagacaacgccgag | attcactctcacaatcagc |
| | gctgcatcc - SEQ ID | aactcagtgcatctgcaaa | agcctgcagcctgaagatt |
| | NO: 317 | tgaacagcctgagaggcga | ttgcaacttattactgtca |
| | caacagcctaataattac | ggacacggctgtttattac | acagcctaataattaccca |
| | ccattcagt - SEQ ID | tgtgcgagagtgggtggaa | ttcagtttcggccctggga |
| | NO: 407 | gctactactactcctacgc | ccaaagtggatgtcaaa - |
| | | tatggacgtctggggccaa | SEQ ID NO: 409 |
| | | gggaccgcggtcaccgtct | |
| | | cctca - SEQ ID NO: | |
| | | 408 | |
| 11D12 | ggattcaccttcagtagt | gaggtgcagttggtggagt | gacatccagttgacccagt |
| | tatgaa - SEQ ID | ctgggggaggcttggtcca | ctccatccttcctgtctgc |
| | NO: 365 | gcctggagggtccctgaga | atctgtaggagacagagtc |
| | attagtagtggtggtagt | ctctcctgtgcagcctctg | accatcacttgctgggcca |
| | tccata - SEQ ID | gattcaccttcagtagtta | gtcaggacattagcagtta |
| | NO: 406 | tgaaatgaactgggtccgc | tttagcctggtatcagcaa |
| | gcgagagtgggtggaagc | caggctccagggaagggac | aaaccagggaaagccccta |
| | tactactactcctacgct | tggagtgggtttcatacat | agctcctgatctatgctgc |
| | atggacgtc - SEQ ID | tagtagtggtggtagttcc | atccactttacaaagtggg |
| | NO: 367 | ataaaatacgcagactctg | gtcccatcaaggttcagcg |
| | caggacattagcagttat - | tgaggggccgattcacctt | gcagtggatctgggacaga |
| | SEQ ID NO: 368 | ctccagagacaacgccgag | attcactctcacaatcagc |
| | gctgcatcc - SEQ ID | aactcagtgtttctccaaa | agcctgcagcctgaagatt |
| | NO: 317 | tgaacagcctgagaggcga | ttgcaacttattactgtca |
| | caacagcctaataattac | ggacacggctgtttattac | acagcctaataattaccca |
| | ccattcagt - SEQ ID | tgtgcgagagtgggggaa | ttcagtttcggccctggga |
| | NO: 407 | gctactactactcctacgc | ccaaagtggatgtcaaa - |
| | | tatggacgtctggggccaa | SEQ ID NO: 411 |
| | | gggaccgcggtcaccgtct | |
| | | cctca - SEQ ID NO: | |
| | | 410 | |
| 14F5 | ggattcacctttagcagc | gaggtgcagttgttggagt | gacatccagatgacccagt |
| | tttgcc - SEQ ID | ctgggggaggcttggtaca | ctccatcctccctgtctgc |
| | NO: 357 | gcctggggggtccctgaga | atctgtaggagacagagtc |
| | attagtgctagtggcggt | ctctcctgtgcagtctctg | accatcacttgccgggcaa |
| | accaca - SEQ ID | gattcacctttagcagctt | gtcagagcattggcaggtt |
| | NO: 412 | tgccatgagctgggtccgc | tttaatttggtatcagcag |
| | gcgaaagaccgatttttg | caggctccagggaagggcc | aaaccagggaaagccccta |
| | gagtgggtagaggggttc | tggagtgggtctcgggtat | aactcctgctttatgctac |
| | gacccc - SEQ ID | tagtgctagtggcggtacc | atccagtttgcaaagtggg |
| | NO: 413 | acaaattacgcagactccg | gtcccagcaaggttcactg |
| | cagagcattggcaggttt - | tgaaaggccggttcaccat | gcagtgggtctgggacaga |
| | SEQ ID NO: 414 | ctccagagacaattccaag | tttcactctcaccatcggc |
| | gctacatcc - SEQ ID | aacacgctctatctgcaaa | agtctgcaacctgaagatt |
| | NO: 361 | tgagcagcctgagagccga | ttgcaacttactactgtca |
| | caacagagttacattacc | ggacacggccgaatattac | acagagttacattaccccg |
| | ccgctcact - SEQ ID | tgtgcgaaagaccgatttt | ctcactttcggcggaggga |
| | NO: 415 | tggagtgggtagaggggtt | ccaaggtggagatcaaa - |
| | | cgacccctggggccaggga | SEQ ID NO: 417 |
| | | atcctggtcaccgtctccc | |
| | | ca - SEQ ID NO: 416 | |
| 15C10 | ggattcacctttagcagc | gaggtgcagttgttggagt | gacatccagatgacccagt |
| | tttgcc - SEQ ID | ctgggggaggcttggtaca | ctccatcctccctgtctgc |
| | NO: 357 | gcctggggggtccctgaga | atctgtaggagacagagtc |
| | attagtgctagtggcggt | ctctcctgtgcagcctctg | accatcacttgccgggcaa |
| | accaca - SEQ ID | gattcacctttagcagctt | gtcagagcattggcaggtt |
| | NO: 412 | tgccatgagctgggtccgc | tttaatttggtatcagcag |
| | gcgaaagaccgatttttg | caggctccagggaagggcc | aaaccagggaaagccccta |
| | gagtgggtagaggggttc | tggagtgggtctcgggtat | agctcctgctctatgctac |
| | gacccc - SEQ ID | tagtgctagtggcggtacc | atccagtttgcaaagtggg |
| | NO: 413 | acaaattacgcagactccg | gtcccagcaaggttcactg |
| | cagagcattggcaggttt - | tgaaaggccggttcaccat | gcagtgggtctgggacaga |
| | SEQ ID NO: 414 | ctccagagacaattccaag | tttcactctcaccatcggc |
| | gctacatcc - SEQ ID | aacacgctctatctgcaaa | agtctgcaacctgaagatt |
| | NO: 361 | tgagcagcctgagagccga | ttgcaacttactactgtca |
| | caacagagttacattacc | ggacacggccgaatattac | acagagttacattaccccg |
| | ccgctcact - SEQ ID | tgtgcgaaagaccgatttt | ctcactttcggcggaggga |
| | NO: 415 | tggagtgggtagaggggtt | ccaaggtggagatcaaa - |
| | | cgacccctggggccaggga | SEQ ID NO: 419 |
| | | atcctggtcaccgtctccc | |
| | | ca - SEQ ID NO: 418 | |

TABLE 6
Nucleotide sequences of antibody framework regions.

| Antibody | |
|---|---|
| Clone | Sequence |
| 1E2 | HFR1 - |
| | caggtgcagctggtggagtctgggggagacttggtcaagcctggagggtccctgagact |
| | ctcctgtgtagcctcg - SEQ ID NO: 420 |
| | HFR2 - atgaactggatccgcctgcctccagggaagggcctggagtgggtttcatac - |
| | SEQ ID NO: 421 |
| | HFR3 - |
| | tactatgcagactctgtgaggggccgattcaccgtctccagggacgacgccaagaattc |
| | actatttctgcacatgaacagcctgagagccgatgacacggccgtttattactgt - |
| | SEQ ID NO: 422 |
| | HFR4 - tggggcccgggaaccctggtcaccgtctcttca - SEQ ID NO: 423 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 |
| | acccgggaatccggggtccctgaccgattcagtggcagcgggtctgggacagatttcac |
| | tctcaccatcaacagcctgcaggctgaagatgtggcagtttattactgt - SEQ ID |
| | NO: 426 |
| | LFR4 - tttggccgggggaccacactggagatcaaa - SEQ ID NO: 427 |
| 1H10 | HFR1 - |
| | caggtgcacctggtggagtctgggggagacttggtcaagcctggagggtccctgagact |
| | ctcctgtgtagcctct - SEQ ID NO: 428 |
| | HFR2 - atgaactggatccgtatgcctccagggaagggcctggagtggatttcatac - |
| | SEQ ID NO: 429 |
| | HFR3 - |
| | tactatgcagactctgtgaagggccgattcaccgtctccagggacgacgccaagaactc |
| | actgtttctacacatgaacaacctgagagccgatgacacggccgtttattactgt - |
| | SEQ ID NO: 430 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - tttggccgggggaccacactggagatcaaa - SEQ ID NO: 427 |
| 2A10 | HFR1 |
| | gaggtgcagctggtgcagtctggagcagaggtgaaaaagcccggggagtctctgaagat |
| | ctcctgtaagggttct - SEQ ID NO: 433 |
| | HFR2 - atcggctgggtgcgccagatgcccgggaaaggcctggagtggatggggatc - |
| | SEQ ID NO: 434 |
| | HER3 |
| | agatacagcccgtccttccaaggccaggtcaccatctcagccgacaagtccatcagtac |
| | cgcctatttgcagtggagcagcctgaaggcctcggacaccgccatgtattactgt - |
| | SEQ ID NO: 435 |
| | HFR4 - tggggccagggaaccctggtcaccgtctcctca - SEQ ID NO: 436 |
| | LFR1 - |
| | gacatccagatgacccagtctccatcctccctgtctgcatctgtcggagacagactcac |
| | catcacttgccgggcaagt - SEQ ID NO: 437 |
| | LFR2 - ttaaattggtatcaacagaacccagggaaagcccctaaggtcctgatttat - |
| | SEQ ID NO: 438 |
| | LFR3 - |
| | agtttggaaagtggggtcccatcaaggttcagtggcagtggatctgggacagatttcac |
| | tctcaccatcagcagtctgcaacctgaagattttgcaacttactactgt - SEQ ID |
| | NO: 439 |
| | LFR4 - ttcggccaagggaccaaggtggaaatcaaa - SEQ ID NO: 440 |
| 2B9 | HFR1 |
| | gaggtgcagcttttggagtcggggggaggcttggcacagccggggaagtccctgagact |
| | ctcctgtgcagcctct - SEQ ID NO: 441 |
| | HFR2 - atgagctgggtccgccaggctcctgggaaggggctggagtgggtctcaggt - |
| | SEQ ID NO: 442 |
| | HFR3 |
| | tactatgcagactccgtgaagggccggttcaccatttccagagacaattccaagagtac |
| | gctgtatcttcaaatgaacagcctgggagccgaggacacggccgtgtattactgt - |
| | SEQ ID NO: 443 |
| | HFR4 - tggggccaagggaccacggtcaccgtctcctca - SEQ ID NO: 444 |
| | LFR1 |
| | gacatccagatgacccagtctccatcctccctgtctgcatctgtaggagacagagtcac |
| | catcacttgccgggcaagt - SEQ ID NO: 445 |
| | LFR2 - ttaaattggtatcagcagaaaccagggaaagcccctaaattcctgatttat - |
| | SEQ ID NO: 446 |
| | LFR3 |
| | agtttgcaaagtggggtcccatcaaggttcagtggcagtggatgtgggantgatttcac |
| | tctcaccatcagcagtctgcaacgtgaagatttcgcagcttactactgt - SEQ ID |
| | NO: 447 |
| | LFR4 - ttcggggggggaccaaggtggagatcaaac - SEQ ID NO: 448 |
| 2F7 | HFR1 - |
| | caggtgcagctggtggagtctgggggaggcttggtcaagcctggagggtccctgagact |
| | ctcctgcgtagcctct - SEQ ID NO: 449 |
| | HFR2 - atgaactggatccgcctgcctccagggagggggctgcagtggatttcatac - |
| | SEQ ID NO: 450 |
| | HFR3 |
| | tactacgcagactctgtgaagggccgattcaccgtctccagggacgacgccaagaattc |
| | actttatctgcaaatgaacagcctgagagccgatgacacggccgtttattactgt - |
| | SEQ ID NO: 451 |
| | HFR4 - tggggccagggaaccctggtcaccgtctcctca - SEQ ID NO: 436 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttaggttggtaccagcagaaagcaggacagcctcctaaactgctcatttac - |
| | SEQ ID NO: 452 |
| | LFR3 - |
| | acccggaattccggggtccctgaccgcttcagtggcagcgggtctgggacagatttcac |
| | tctcaccatcagcagcctgcaggctgaggatgtggcagtttattactgt - SEQ ID |
| | NO: 453 |
| | LFR4 - tttggccaggggaccaacctggagatcaaa - SEQ ID NO: 454 |
| 2F11 | HFR1 - |
| | gaggtgcagctggtgcagtctggagcagaggtgaaaaagcccggggagtctctgaagat |
| | ctcctgtaagggttct - SEQ ID NO: 433 |
| | HFR2 - atcggctgggtgcgccagatgcccgggaaaggcctggagtggatggggatc - |
| | SEQ ID NO: 434 |
| | HFR3 - |
| | agatacagcccgtccttccaaggccaggtcaccctctcagccgacaagtccatcagtac |
| | cgcctacctacagtggaacagcctgaaggcctcggacaccgccatgtattactgt - |
| | SEQ ID NO: 455 |
| | HFR4 - tggggccagggaaccctggtcaccgtcgcctcat - SEQ ID NO: 456 |
| | LFR1 - |
| | gacatccagatgacccagtctccatcctccctgtctgcatctgtaggagacagagtcac |
| | catcacttgccgggcaagt - SEQ ID NO: 445 |
| | LFR2 - ttaaattggtatcagcagaaaccagggacagcccctaaactcctgatttat - |
| | SEQ ID NO: 457 |
| | LFR3 - |
| | agtttgcaaggtggggtcccatcaaggttcagtggcagtggctctgggacagatttcac |
| | tctcaccatcagcagtctgcaacctgaagattttgcaacttactactgt - SEQ ID |
| | NO: 458 |
| | LFR4 - ttcggccaagggaccaaggtggaaatcaaa - SEQ ID NO: 440 |
| 2H1 | HER1 - |
| | caggtgcagctggtggagtctgggggagacttggtcaagcctggagggtccctgagact |
| | ctcctgtgtagcctct - SEQ ID NO: 459 |
| | HFR2 - atgaactggatccgcctgcctccagggaagggcctggagtggatttcctac |
| | - SEQ ID NO: 460 |
| | HFR3 - |
| | tactatgcagactctgtgaagggccgattcaccgtctccagggacgacgccaagaactc |
| | actatttctgcacatgaacagcctgagagccgatgacacggccgtttattactgt - |
| | SEQ ID NO: 461 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaaacaggacagcctcctaagttgctcattttc - |
| | SEQ ID NO: 462 |
| | LFR3 - |
| | acccgggaatccggggtccctgaccgattcagtggcagcgggtctgggacagatttcac |
| | tctcaccatcagcagcctgcaggctgaggatgtggcagtttattactgt - SEQ ID |
| | NO: 463 |
| | LFR4 - tttggccgggggaccacactggagatcaaa - SEQ ID NO: 427 |
| 4E6 | HFR1 - |
| | gaagtgcagttggtggagtctgggggagacttggtacagcctggcaggtccctgagact |
| | ctcctgtgcagcctct - SEQ ID NO: 464 |
| | HFR2 - atgcactgggtccggcaaggtccagggaagggcctggagtgggtctcaggc - |
| | SEQ ID NO: 465 |
| | HFR3 - |
| | ggctatgcggactctgtgaagggccgattcaccatttccagagacaacgccaagaactc |
| | cctgtacctgcgaatgaacagtctgagagctgacgacacggccttctattactgt - |
| | SEQ ID NO: 466 |
| | HFR4 - tggggccacgggacaatggtcaccgtctcttca - SEQ ID NO: 467 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttggattggtatcagcagaaaccaggacagcctcctaagctgctcatttat - |
| | SEQ ID NO: 468 |
| | LFR3 |
| | tcccgggaatccggggtccctgaccgattcagtggcagcgggtctgggacagatttcac |
| | tctgaccatcagcagcctgcaggctgaagatgtggcagtttattactgt - SEQ ID |
| | NO: 469 |
| | LFR4 - ttcggcggagggaccaaggtggagatcaaa - SEQ ID NO: 470 |
| 4F7 | HFR1 - |
| | caggtgcagctggtggagtctgggggagacttggtcaagcctggagggtccctgagact |
| | ctcctgtgtagcctct - SEQ ID NO: 459 |
| | HFR2 - atgaactggatccgcctgcctccagggaagggcctggagtggatttcatac |
| | - SEQ ID NO: 471 |
| | HFR3 - |
| | tactatgcagactctgtgaagggccgattcaccgtctccagggacgacgccaagaactc |
| | actatttctacacatgaacaacctgagagccgatgacacggccgtttattactgt - |
| | SEQ ID NO: 472 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 - |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - tttggccgggggaccacactggagatcaaa - SEQ ID NO: 427 |
| 5A8 | HFR1 - |
| | caggtgcagctggtggagtctgggggagacttggtcaagcctggagggtccctgagact |
| | ctcctgtgtagcctct - SEQ ID NO: 459 |
| | HER2 - atgaactggatccgcctgcctccagggaggggcctggagtggatttcatac - |
| | SEQ ID NO: 473 |
| | HFR3 |
| | tactatgcagactctgtgaagggccgattcaccgtctccagggacgacgccaagaagtc |
| | actatttctgtacatgaacagcctgagagccgatgacacggccgtttattactgt - |
| | SEQ ID NO: 474 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - tttggccgggggaccacactggagatcaaa - SEQ ID NO: 427 |
| 6E1 | HFR1 - |
| | gaggtgcagttgttggagtctgggggaggcttggtacagcctggggggtccctgagact |
| | ctcctgtgcagcctct - SEQ ID NO: 475 |
| | HFR2 - atgagctgggtccgccaggctccagggaaggggctggagtgggtctcaggt - |
| | SEQ ID NO: 476 |
| | HFR3 - |
| | tactacgcagactccgtgaagggccggttcaccatctccagagacaattccaagaacac |
| | gctgtatctgcaaatgaacagcctgagagccgaggacacggccatatattactgt - |
| | SEQ ID NO: 477 |
| | HFR4 - tggggccaagggacgatggtcaccgtctcttca - SEQ ID NO: 478 |
| | LFR1 - |
| | gacatccagatgacccagtctccgtcctccctgtctgcatctgtaggaggcagagtcac |
| | catcacttgccggacaagt - SEQ ID NO: 479 |
| | LFR2 - ttaaattggtatcagcagaaagcagggaaagcccctaaactcctgatctat - |
| | SEQ ID NO: 480 |
| | LFR3 - |
| | aatttgcaaagtggggtcccatcaaggttcactggcagtggctctgggacagatttcac |
| | tctcaccatcagcagtctgcaacctgaagattttgcagcttactactgt - SEQ ID |
| | NO: 481 |
| | LFR4 - ttcggccctgggaccaaagtggctttcaaa - SEQ ID NO: 482 |
| 6H12 | HFR1 - |
| | gaggtgcagttggtggagtctgggggaggcttggtccagcctggagggtccctgagact |
| | ctcctgtgcagcctct - SEQ ID NO: 483 |
| | HFR2 - atgaactgggtccgccaggctccagggaagggactggagtgggtttcatac - |
| | SEQ ID NO: 484 |
| | HFR3 - |
| | aaatacgcagactctgtgaggggccgattcgccttctccagagacaacgccgagaactc |
| | agtgcatctgcaaatgaacagcctgagagccgaggacacggctgtttattactgt - |
| | SEQ ID NO: 485 |
| | HFR4 - tggggccaagggaccgcggtcaccgtctcctca - SEQ ID NO: 486 |
| | LFR1 - |
| | gacatccagttgacccagtctccatccttcctgtctgcatctgtaggagacagagtcac |
| | catcacttgctgggccagt - SEQ ID NO: 487 |
| | LFR2 - ttagcctggtatcagcaaaaaccagggaaagcccctaagctcctgatctat - |
| | SEQ ID NO: 488 |
| | LFR3 - |
| | actttgcaaagtggggtcccatcaaggttcaccggcagtggatctgggacagaattcac |
| | tctcacaatcagcagcctgcagcctgaagattttgcaacttattactgt - SEQ ID |
| | NO: 489 |
| | LFR4 - ttcggccctgggaccaaagtggatgtcaaa - SEQ ID NO: 490 |
| 7A4 | HFR1 - |
| | caggtgcagctggtggagtctgggggagacttggtcaagcctggagggtccctgagact |
| | ctcctgtgtagcctct - SEQ ID NO: 459 |
| | HFR2 - atgaactggatccgcctgcctccagggaagggcctggagtggatttcatac - |
| | SEQ ID NO: 471 |
| | HFR3 - |
| | tactatgcagactctgtgaagggccgattcaccgtctccagggacgacgccaagaactc |
| | actatttctgcacatgaacagcctgagagccgatgacacggccgtttattactgt - |
| | SEQ ID NO: 461 |
| | HFR4 - tggggcccgggaaccctggtcaccgtctcctcc - SEQ ID NO: 491 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 - |
| | attcgggaatccggggtccctgaccgattcagtggcagcgggtctgggacagatttcac |
| | tctcaccatcagcagcctgcaggctgaagatgtggcagtttattattgt - SEQ ID |
| | NO: 492 |
| | LFR4 - tttggccgggggaccacactggagatcaaa - SEQ ID NO: 427 |
| 7E5 | HFR1 - |
| | gaggtgcagctgttggaatctgggggaggcctgatacaaccgggggggtccctgagact |
| | ctcctgtgcagcctct - SEQ ID NO: 493 |
| | HFR2 - atgagctgggtccgccaggctcctgggaaggggctggagtgggtctcaggt - |
| | SEQ ID NO: 442 |
| | HFR3 - |
| | tactatggagactccgtgaagggccggttcaccatttccagagacaattccaagagtac |
| | gctgtatctgcaaatgaacagcctgagagacgcggacacggccgtatattattgt - |
| | SEQ ID NO: 494 |
| | HFR4 - tggggccaagggaccacggtcaccgtctcctca - SEQ ID NO: 444 |
| | LFR1 - |
| | gacatccagatgacccagtctccatcctccctgtctgcatccgtaggagacagagtcac |
| | catcacttgccgggcaagt - SEQ ID NO: 495 |
| | LFR2 - ttaaattggtatcagcagaagccagggaaagcccctaagttcctgatttat - |
| | SEQ ID NO: 496 |
| | LFR3 - |
| | agtttgcaaagtggggtcccatcaaggttcagtggcagtggatgtgggacagatttctc |
| | tctcaccatcagcagtntgcaccctggagacttcgcaacttactactgt - SEQ ID |
| | NO: 497 |
| | LFR4 - ttcggcggagggaccaaggtggagatcaaa - SEQ ID NO: 470 |
| 7H8 | HFR1 - |
| | gaggtgcagttgttggagtctgggggaggcttggtacagcctggggggtccctgagact |
| | ctcctgtgcagcctct - SEQ ID NO: 475 |
| | HFR2 - atgacctgggtccgccaggctccagggaagggactggagtgggtctcaggt - |
| | SEQ ID NO: 498 |
| | HFR3 |
| | tactacgcagactccgtgaagggccggttcaccatttccagagacaattccaagagcac |
| | gctctatctgcaaatgaacagtctgagagccgaggacacggccgtatattactgt - |
| | SEQ ID NO: 499 |
| | HFR4 - tggggccagggaaccctggtcaccgtctcctca - SEQ ID NO: 436 |
| | LFR1 - |
| | gacatccagatgacccagtctccttccaccctgtctgcatctgtaggagacagagtcac |
| | catcacttgccgggccagt - SEQ ID NO: 500 |
| | LFR2 - ttggcctggtatcagcagaaaccagggaaagcccctaaactcctgatctat - |
| | SEQ ID NO: 501 |
| | LFR3 - |
| | agtttagaaagtggggtcccatcaaggttcagcggcagtggatctgggacagatttcac |
| | tctcaccatcagcagcctgcagcctgatgattttgcaagttattactgc - SEQ ID |
| | NO: 502 |
| | LFR4 - tttggccaggggaccaagctggagatcaca - SEQ ID NO: 503 |
| 8A11 | HFR1 - |
| | caggtgcagttggtggagtctgggggagacttggtcaagcctggagggtccctgagact |
| | ctcctgtgtagcctct - SEQ ID NO: 504 |
| | HFR2 - atgaactggatccgcctgcctccagggaagggcctggagtggatttcatac - |
| | SEQ ID NO: 471 |
| | HFR3 - |
| | tactatgcagactctgtgaagggccgattcaccgtctccagggacgacgccaagaattc |
| | actatttctgcacatgaacagtctgagagccgaggacacggccgtttattactgt - |
| | SEQ ID NO: 505 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 - |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - tttggccgggggaccacactggagatcaaa - SEQ ID NO: 427 |
| 8D10 | HFR1 - |
| | caggtgcacctggtggagtctgggggagacttggtcaagcctggagggtccctgagact |
| | ctcctgtgtagcctct - SEQ ID NO: 428 |
| | HFR2 - atgaactggatccgcctgcctccagggaagggcctggagtggatttcatac - |
| | SEQ ID NO: 471 |
| | HFR3 - |
| | tactatgcagactctgtgaagggccgattcaccgtctccagggacgacgccaagaactc |
| | aatgtttctgcacatgaacagcctgagagccgatgacacggccgtttattactgt - |
| | SEQ ID NO: 506 |
| | HFR4 - tggggcccgggaaccctggtcaccgtctcctca - SEQ ID NO: 431 |
| | LFR1 - |
| | gacatcgtgatgacccagtctccagactccctggctgtgtctctgggcgagagggccac |
| | catcaactgcaagtccagc - SEQ ID NO: 424 |
| | LFR2 - ttagcttggtaccagcagaaagcaggacagcctcctaagttgctcattttc - |
| | SEQ ID NO: 425 |
| | LFR3 - |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - tttggccgggggaccacactggagatcaaa - SEQ ID NO: 427 |
| 9B4 | HFR1 |
| | caggtgcaactggtggagtctgggggagacttggtcaagcctggagggtccctgagact |
| | ctcctgtgtagcctct - SEQ ID NO: 507 |
| | HFR2 - atgaactggatccgcctgcctccagggaagggcctggagtggatttcatac - |
| | SEQ ID NO: 471 |
| | HFR3 |
| | tactatgcagactctgtgaaggggcgattcaccgtctccagggacgacgccaagaactc |
| | attatttctgcacatgaacagcctgagagccgatgacacggccgtttattactgt - |
| | SEQ ID NO: 508 |
| | HFR4 - tggggcccgggaaccctggtcaccgtctcctca - SEQ ID NO: 431 |
| | LFR1 - |
| | gacatcgtgatgacccagtctccagactccctggctctgtctctgggcgagagggccac |
| | cattaactgcaagtccagc - SEQ ID NO: 509 |
| | LFR2 - ttagcttgttaccaacagaaagcaggacagcctcctaagttgctcattttc - |
| | SEQ ID NO: 510 |
| | LFR3 |
| | acccgggaatccggggtccctgaccgattcagtggcagcgggtctgggacagatttcac |
| | tctcaccatcagcagcctgcaggctgaagatgtggcagtttattactgt - SEQ ID |
| | NO: 432 |
| | LFR4 - ttcggccgggggaccacactggagatcaaa - SEQ ID NO: 511 |
| 9G9 | HFR1 |
| | gaggtgcagctgttggagtctgggggaggcttggtacagcctggggggtccctgagact |
| | ctcctgtgcagcctct - SEQ ID NO: 512 |
| | HFR2 - atgagctgggtccgccaggctccagggaaggggctggagtgggtctcaggt - |
| | SEQ ID NO: 476 |
| | HER3 - |
| | tactacgcagactccgtgaagggccggttcaccatttccagagacaattccaagaacac |
| | gctgtatctgcaaatgaacagcctgagagccgacgacacggccttatattactgt - |
| | SEQ ID NO: 513 |
| | HER4 - tggggccagggaaccctggtcaccgtctcctca - SEQ ID NO: 436 |
| | LFR1 - |
| | gacatccagatgacccagtctccatcctccctgtctgcatctgtaggagacagagtcac |
| | catcacttgccgggcaagt - SEQ ID NO: 445 |
| | LFR2 - ttaatttggtatcagcagaaaccagggaaagcccctaagctcctgatttat - |
| | SEQ ID NO: 514 |
| | LFR3 - |
| | agtttgcaaagtggggtcccatcaaggttcagtggcagtggatctgggacagatttcac |
| | tctcattatcagcagtctgcaacctgaagattttgcaacttattactgt - SEQ ID |
| | NO: 515 |
| | LFR4 - ttcggcggagggaccaaagtggagatcaaa - SEQ ID NO: 516 |
| 9H9 | HER1 - |
| | gaggtgcagttggtggagtctgggggaggcttggtccagcctggagggtccctgagact |
| | ctcctgtgcagcctct - SEQ ID NO: 483 |
| | HFR2 - atgaactgggtccgccaggctccagggaagggactggagtgggtttcatac - |
| | SEQ ID NO: 484 |
| | HFR3 - |
| | aaatacgcagactctgtgaggggccgattcaccttctccagagacaacgccgagaactc |
| | agtgcatctgcaaatgaacagcctgagaggcgaggacacggctgtttattactgt - |
| | SEQ ID NO: 517 |
| | HFR4 - tggggccaagggaccgcggtcaccgtctcctca - SEQ ID NO: 486 |
| | LFR1 - |
| | gacatccagttgacccagtctccatccttcctgtctgcatctgtaggagacagagtcac |
| | catcacttgctgggccagt - SEQ ID NO: 487 |
| | LFR2 - ttagcctggtatcagcaaaaaccagggaaagcccctaagctcctgatttat - |
| | SEQ ID NO: 518 |
| | LFR3 - |
| | actttgcaaagtggggtcccatcaaggttcagcggcagtggatntgggacagaattcac |
| | tctcacaatcagcagcttgcagcgtgaagattttgcaacttattactgt - SEQ ID |
| | NO: 519 |
| | LFR4 - ttcggccctgggaccaaagtggatgtcaaa - SEQ ID NO: 490 |
| 11D12 | HFR1 - |
| | gaggtgcagttggtggagtctgggggaggcttggtccagcctggagggtccctgagact |
| | ctcctgtgcagcctct - SEQ ID NO: 483 |
| | HFR2 - atgaactgggtccgccaggctccagggaagggactggagtgggtttcatac - |
| | SEQ ID NO: 484 |
| | HFR3 - |
| | aaatacgcagactctgtgaggggccgattcaccttctccagagacaacgccgagaactc |
| | agtgtttctccaaatgaacagcctgagaggcgaggacacggctgtttattactgt - |
| | SEQ ID NO: 520 |
| | HFR4 - tggggccaagggaccgcggtcaccgtctcctca - SEQ ID NO: 486 |
| | LFR1 - |
| | gacatccagttgacccagtctccatccttcctgtctgcatctgtaggagacagagtcac |
| | catcacttgctgggccagt - SEQ ID NO: 487 |
| | LFR2 - ttagcctggtatcagcaaaaaccagggaaagcccctaagctcctgatctat - |
| | SEQ ID NO: 488 |
| | LFR3 - |
| | actttacaaagtggggtcccatcaaggttcagcggcagtggatctgggacagaattcac |
| | tctcacaatcagcagcctgcagcctgaagattttgcaacttattactgt - SEQ ID |
| | NO: 521 |
| | LFR4 - ttcggccctgggaccaaagtggatgtcaaa - SEQ ID NO: 490 |
| 14F5 | HFR1 - |
| | gaggtgcagttgttggagtctgggggaggcttggtacagcctggggggtccctgagact |
| | ctcctgtgcagtctct - SEQ ID NO: 522 |
| | HFR2 - atgagctgggtccgccaggctccagggaagggcctggagtgggtctcgggt - |
| | SEQ ID NO: 523 |
| | HFR3 |
| | aattacgcagactccgtgaaaggccggttcaccatctccagagacaattccaagaacac |
| | gctctatctgcaaatgagcagcctgagagccgaggacacggccgaatattactgt - |
| | SEQ ID NO: 524 |
| | HFR4 - tggggccagggaatcctggtcaccgtctcccca - SEQ ID NO: 525 |
| | LFR1 - |
| | gacatccagatgacccagtctccatcctccctgtctgcatctgtaggagacagagtcac |
| | catcacttgccgggcaagt - SEQ ID NO: 445 |
| | LFR2 - ttaatttggtatcagcagaaaccagggaaagcccctaaactcctgctttat - |
| | SEQ ID NO: 526 |
| | LFR3 |
| | agtttgcaaagtggggtcccagcaaggttcactggcagtgggtctgggacagatttcac |
| | tctcaccatcggcagtctgcaacctgaagattttgcaacttactactgt - SEQ ID |
| | NO: 527 |
| | LFR4 - ttcggcggagggaccaaggtggagatcaaa - SEQ ID NO: 470 |
| | HFR1 - |
| | gaggtgcagttgttggagtctgggggaggcttggtacagcctggggggtccctgagact |
| | ctcctgtgcagcctct - SEQ ID NO: 475 |
| | HFR2 - atgagctgggtccgccaggctccagggaagggcctggagtgggtctcgggt - |
| | SEQ ID NO: 523 |
| | HER3 - |
| | aattacgcagactccgtgaaaggccggttcaccatctccagagacaattccaagaacac |
| | gctctatctgcaaatgagcagcctgagagccgaggacacggccgaatattactgt - |
| | SEQ ID NO: 524 |
| 15C10 | HFR4 - tggggccagggaatcctggtcaccgtctcccca - SEQ ID NO: 525 |
| | LFR1 - |
| | gacatccagatgacccagtctccatcctccctgtctgcatctgtaggagacagagtcac |
| | catcacttgccgggcaagt - SEQ ID NO: 445 |
| | LFR2 - ttaatttggtatcagcagaaaccagggaaagcccctaagctcctgctctat - |
| | SEQ ID NO: 528 |
| | LFR3 - |
| | agtttgcaaagtggggtcccagcaaggttcactggcagtgggtctgggacagatttcac |
| | tctcaccatcggcagtctgcaacctgaagattttgcaacttactactgt - SEQ ID |
| | NO: 527 |
| | LFR4 - ttcggcggagggaccaaggtggagatcaaa - SEQ ID NO: 470 |

### Sequences

BK virus VP1 protein Accession No. P03088 SEQ ID NO: 529

MAPTKRKGECPGAAPKKPKEPVQVPKLLIKGGVEVLEVKTGVDAIT
EVECFLNPEMGDPDENLRGESLKLSAENDFSSDSPERKMLPCYST
ARIPLPNLNEDLTCGNLLMWEAVTVQTEVIGITSMLNLHAGSQKV
HEHGGGKPIQGSNFHFFAVGGEPLEMQGVLMNYRSKYPDGTITPK
NPTAQSQVMNTDHKAYLDKNNAYPVECWVPDPSRNENARYFGTFT
GGENVPPVLHVTNTATTVLLDEQGVGPLCKADSLYVSAADICGLF
TNSSGTQQWRGLARYFKIRLRKRSVKNPYPISFLLSDLINRRTQR
VDGQPMYGMESQVEEVRVEDGTERLPGDPDMIRYIDKQGQLQTKM
L

### EXAMPLES

### Example 1

### INTRODUCTION

BK virus is a member of the polyoma virus family and is most closely related to JC virus, the causative agent of progressive multifocal leukoencephalopathy (PML). BK is a non-enveloped ds DNA virus with three functional regions: early viral gene region (EVGR) that encodes the large and small T cell antigens, the late viral gene region (LVGR) encoding the capsid proteins VP1, VP2 and VP3 and the non-structural agnoprotein and the non-coding control region (NCCR). The capsid protein, VP1, found in both viruses and virus like particles (VLP) is essential for target cell binding and is the target of neutralizing antibodies (2). BK virus infection occurs in approximately 80-90% of the population with most occurring early in life (2). Following primary infection, the virus is largely asymptomatic in individuals with intact immunity and resides in the renal urinary tract as a persistent or latent infection. The virus is not detectable in the blood but in some individuals, virus is excreted in the urine (viruria). In immune suppressed patients 30-60% secrete virus into urine, half of these at very high levels (>7 log₁₀ c/ml). Half of the high viruria patients develop viremia (BK DNA in plasma). PyVAN following kidney transplantation and polyomavirus-associated hemorrhagic cystitis (PyVHC) following allogeneic hematopoietic stem cell transplantation represent unmet medical needs associated with reactivation of BK viruses. BK virus replication occurs in 30-50% of kidney transplant patients with 7-8% leading to graft dysfunction and/or loss (3). BKPyV in blood represents the primary method for diagnosis and levels of BKPyV-DNA greater than 10³ particles per ml suggests probable PyVAN while above 10⁴/ml it is presumed (2, 4, 5). When such levels of viral DNA are observed along with changes in kidney function the only treatment is lowering of immunosuppressive therapy (5). In hematopoietic stem cell transplantation patients, 10-25% develop PyVHC leading to pain, bleeding, obstruction and in some cases, renal failure (6, 7).

Several factors have been reported to predispose a recipient to develop PyVAN. High urine levels of virus or high serum antibody titers to the virus in the donor are risk factors for the development of PyVAN in the recipient (3, 8-10). There are four genotypes of the virus found in the population, I, II, III and IV with I the most prevalent (60-80%) and IV (10-20%) next most common. These genotypes/serotypes are serological distinct (11). The genotype of the donor is responsible for infection in most instances (2). The early appearance of BK replication/viremia increases the risk of acute graft rejection (12). Importantly, if the graft recipient lacks antibodies against the viral genotype found in the donor organ (8-10) or there is a mismatch in viral genotype between donor and recipient, the risk of developing viremia and PyVAN is increased (2, 3). The combination of high antibody titers in donor (indicating high viral load) and low or mismatched antibody titers in recipient is a significant risk factor (3, 8). In a prospective study of 168 transplant patients by Solis et al (3) the authors showed that high neutralizing antibody titer against the donor strain led to a very significant reduction in risk of viral BK viremia and each log₁₀ rise in antibody titer reduced the risk by 50%. The observations that genotype mismatch increased the risk significantly support a primary role for antibodies in prevention of disease and argue that monoclonal antibodies may be utilized prophylactically/therapeutically to lessen the risk of PyVAN (2, 3, 8). The goal of this study was to produce mAbs capable of neutralizing all four genotypes of BK virus or find combinations that could achieve the same.

### Results

### Generation of BK Virus Neutralizing Monoclonal Antibodies

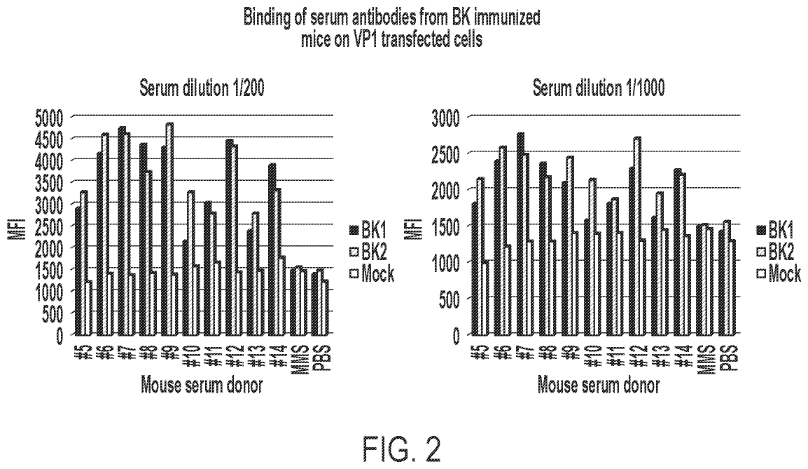

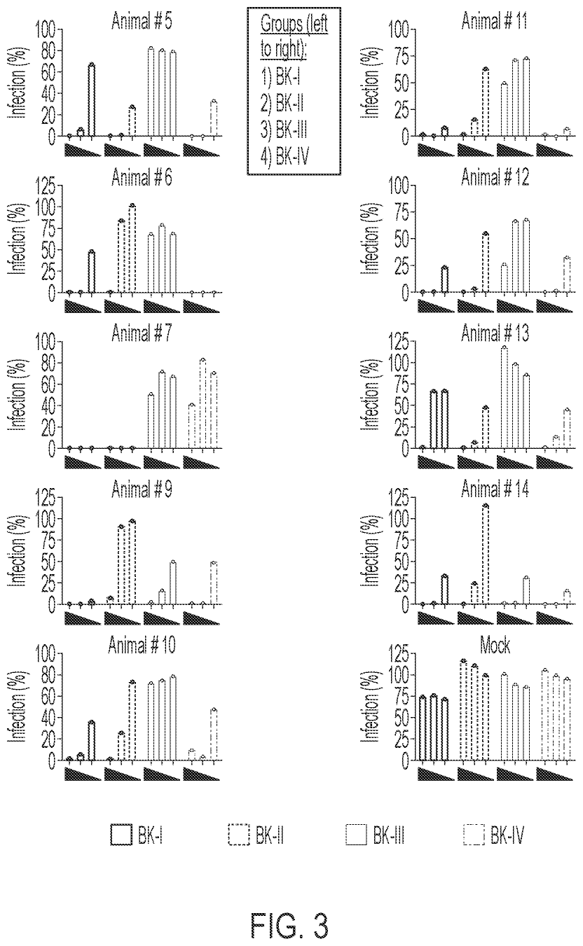

Immunization and serum testing: To generate mAb against BK virus, VelocImmune mice (13) were immunized with various combination of cDNAs encoding for the VP1 protein of the diverse BKV genotypes (Table 7). In general, three immunization regimens were utilized to develop antibodies to diverse BK serotypes. Veloclmmune mice encode for chimeric immunoglobulins consisting of human Vh and Vl chains and a mouse Fc domain. They can be made fully human by a simple cloning step (13). The immunization strategy of utilizing different VP1 genotypes cDNA was to enrich for broadly neutralizing mAbs against different BKV genotypes. Following immunization, the sera from the respective animals were collected and analyzed by flow cytometry on EXPI293F cells transfected with the VP1. Serum binding to VP1 from BK1 and BK4 is shown in FIG. 2. Sera from the animals was subsequently tested for neutralization of pseudoviruses generated with the VP1 from genotypes I, II, III and IV (FIG. 3).

TABLE 7
Immunization strategy to generate BK virus
neutralizing monoclonal antibodies

| | Primary- | Secondary- | | Boost- | |
|---|---|---|---|---|---|
| | genotype | genotype | Tertiary- | pseudovirus | |
| | DNA | DNA | DNA | preps | |
| Group | BKI | BKIV | BKIV | PSV I, IV | Fusion |
| 1 | | | | | 1, 2 |
| Group | BKI, | BKI, BKIV | BKI, BKII, | PSV I, IV | Fusion |
| 2 | BKIV | | BKIII | | 3 |
| Group | BKII, | BKII, BIII | BKI, BKIV | PSV I, II, | Fusion |
| 3 | BKIII | | BKI alone | III, IV | 4 |

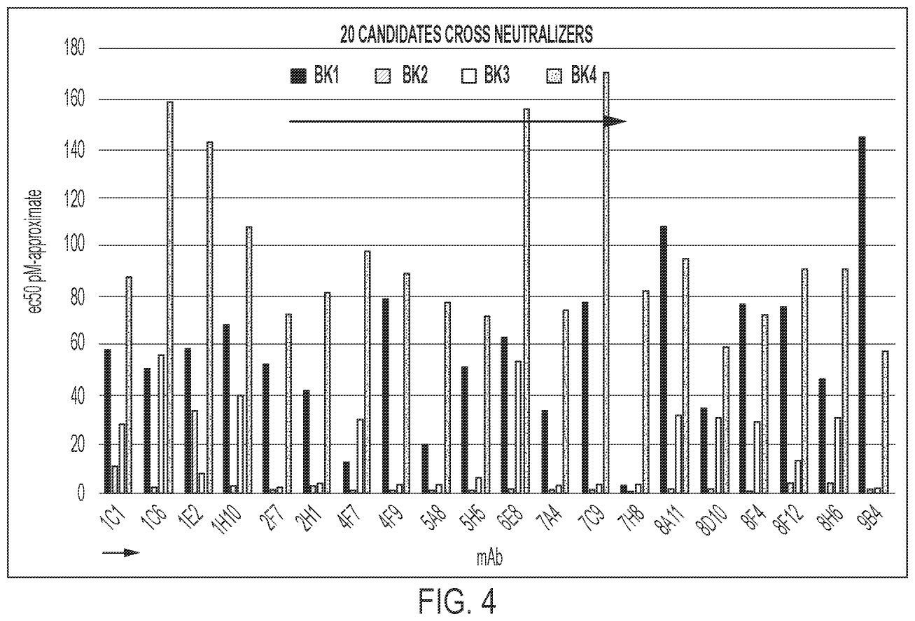

Generation of hybridomas: The immunized mice whose serum demonstrated a robust cross-neutralizing activity against diverse BK pseudoviruses were selected for the generation of hybridomas. The spleens from animals 9, 12, and 14 were fused to Sp2/0 cells, plated in culture dishes with selective media and individual clones collected 12-14 days later using the Hamilton/Stem Cell EzPick. Two days later supernatants were screened by flow cytometry on BK-VP1 transfected 293TT cells transfected with each of the four VP1 and positive clones were further evaluated in a pseudovirus neutralization assay against BK I, II, III, and IV genotypes. Antibodies binding and neutralizing all four genotypes, 3, 2 or a single genotype were identified. Neutralization of pseudovirus was performed as follows: Upper portion of the table shows responses of selection of supernatants tested for both neutralizing and binding to the VP1 of each of the four genotypes. Numbers in tables for neutralization represent percent of infected cells with antibody treatment. Numbers in table for flow cytometry represent MFI of antibody binding to cells transfected with BK I-IV VP I or mock transfected (median). In the lower portion of the table, the supernatants are grouped by the similarities of their binding profiles. These results are performed with undiluted culture supernatant with unknown protein concentration. These results represent hybridoma supernatants of unknown concentration at a single dilution. Hybridomas producing antibodies shown to have activity in the preliminary screening were grown and the approximate concentration of antibody was measured using biolayer label-free protein interferometry on an Octet Red 96 compared to isotype matched standard controls. Based upon the predicted protein concentrations the supernatants were tested for pseudovirus neutralization (FIG. 4) and approximate EC50 determined.

### Selection of Broadly Cross Neutralizing Antibodies:

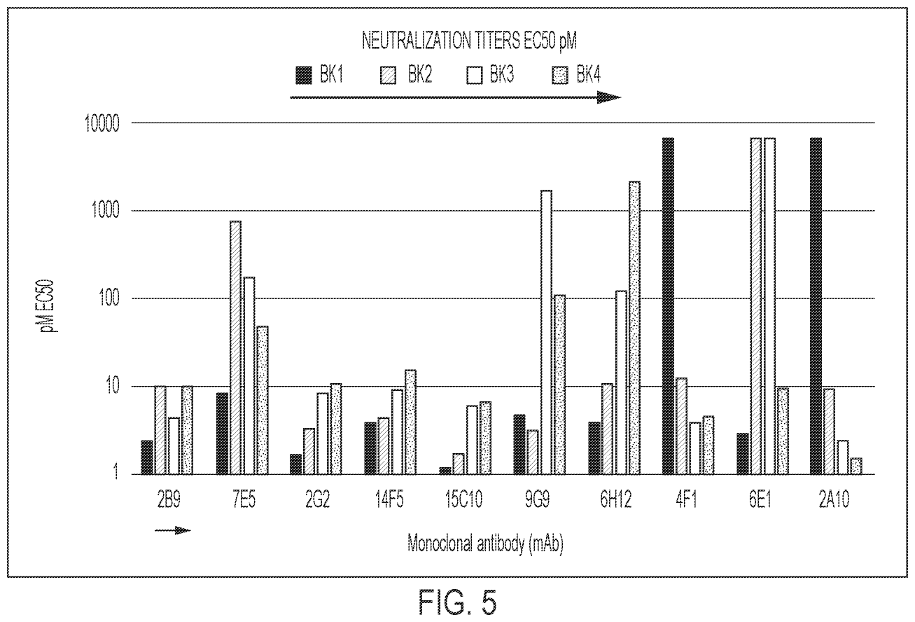

While many of the supernatants inhibited infection of BK I, II, III, and IV genotype pseudoviruses, in subsequent experiments using purified immunoglobulin many were unable to effectively neutralize all four genotypes efficiently when tested at concentrations below 200 ng/ml. The isotypes of all the positive clones were determined and antibodies of the IgG class were purified and tested for neutralization of all four genotypes and EC50 values calculated for each. FIG. 5 shows the EC50 values of a panel of purified cross neutralizing antibodies from three separate fusions. Several mAbs show broad cross neutralization at low pM concentrations while others neutralize two or three of the genotypes. An EC50 above 1000 is considered non-neutralizing.

Comparison of Neutralization with a Known Therapeutic Candidate Antibody

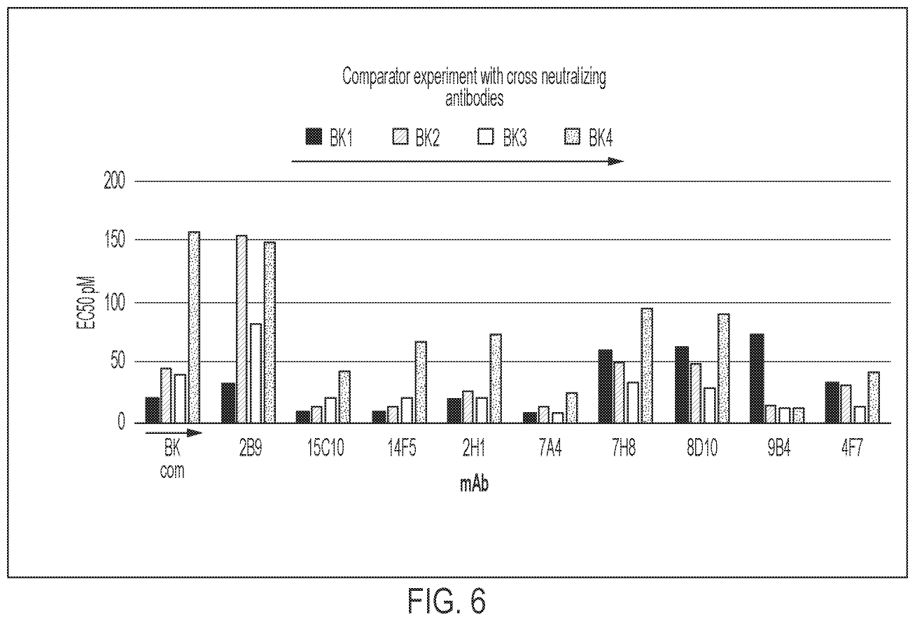

Purified monoclonal antibodies that neutralized all four genotypes were compared in pseudoviral neutralization assays to a control mAb known to be a therapeutic candidate (BK comparator) (FIG. 6). Most of the mAb neutralize as well or belier than the comparator mAb.

List of Candidate mAbs that Neutralize BK Virus

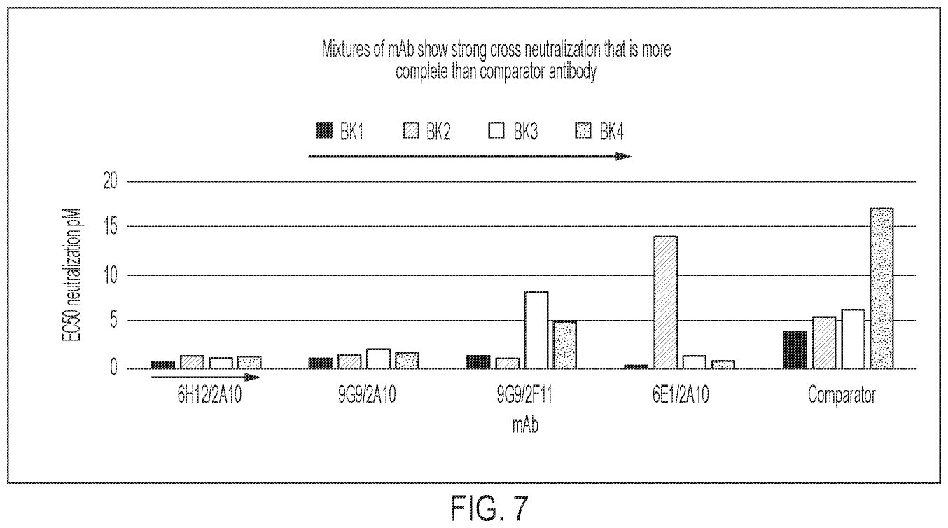

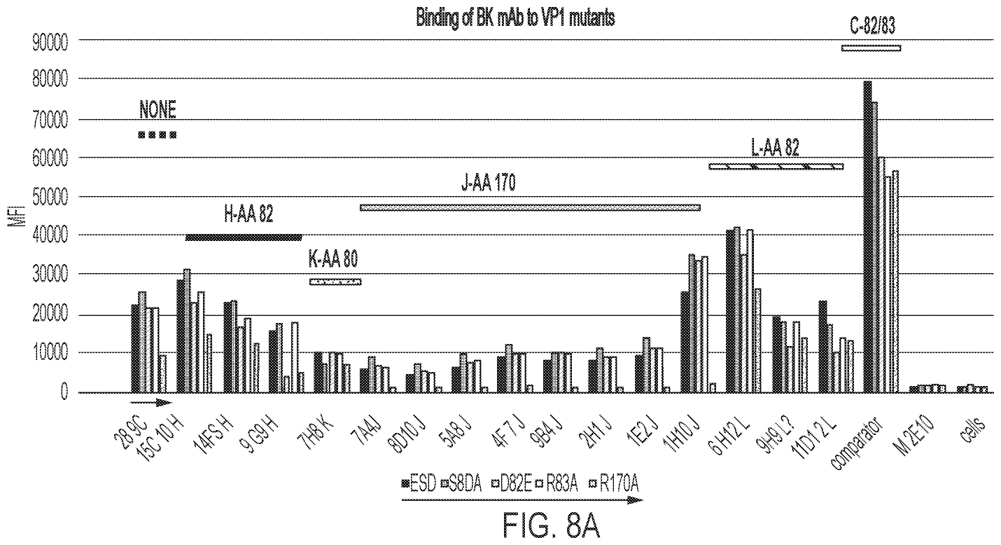

Table 8 shows a summary of mAb collected from four different fusions that neutralize some or all the BK genotypes as measured by pseudovirus assays (also see FIG. 7). The antibodies are all primarily IgG2a and IgG2b with a single IgG1. They represent 9 unique sequence families. All listed antibodies are unique within their sequence family. The EC50 of the comparator antibody is included for comparison.

TABLE 8
Summary of the neutralizing antibodies generated against BK viruses

| | BK1 | BK2 | BK3 | BK4 | Sequence Group | ISOTYPE | Fusion number |
|---|---|---|---|---|---|---|---|
| CROSS NEUTRALIZERS | | | | | | | |
| 2B9 | 32.2 | 154 | 80.8 | 148.2 | C | IgG2b | 1 |
| 7E5 | 24.9 | 1162 | 414.9 | 133 | C | IgG2a | 1 |
| 14F5 | 10.1 | 13.1 | 21.5 | 67.1 | H | IgG2b | 3 |
| 15C10 | 9.4 | 13.1 | 21.1 | 42.3 | H | IgG2b | 3 |
| 7A4 | 9.1 | 13.2 | 8.1 | 25.1 | J | IgG2a | 4 |
| 2H1 | 20.2 | 26.1 | 21.9 | 72.8 | J | IgG2a | 4 |
| 7H8 | 59.6 | 50 | 33.4 | 94.3 | K | IgG2a | 4 |
| 8D10 | 62.9 | 49.2 | 29 | 89.4 | J | IgG2a | 4 |
| 9B4 | 72.5 | 15.2 | 11.8 | 11.8 | J | IgG1 | 4 |
| 4F7 | 32.5 | 31.9 | 14.3 | 41.6 | J | IgG2a | 4 |
| 2F7 | 52.5 | 1.1 | 2.2 | 72.4 | J | IgG2a | 4 |
| 8A11 | 108 | 1.4 | 31.5 | 94.8 | J | IgG2a | 4 |
| 5A8 | 20.1 | 0.9 | 3.9 | 77.4 | J | IgG2a | 4 |
| 2H1 | 41.8 | 2.8 | 3.4 | 81.5 | J | IgG2a | 4 |
| 1E2 | 59 | 33.2 | 8.1 | 142.7 | J | IgG2a | 4 |
| 1H10 | 68.4 | 2.8 | 39.7 | 107.3 | J | IgG2a | 4 |
| Comparator | 20.2 | 45.1 | 40.2 | 156.3 | | | |
| PARTIAL NEUTRALIZERS-separate experiment | | | | | | | |
| 2F11 | >6667 | 83.8 | 10.5 | 6.1 | E | IgG2a | 2 |
| 6E1 | 2.95 | >6667 | >6667 | 9.5 | G | IgG2a | 2 |
| 2A10 | >6667 | 9.4 | 2.4 | 1.5 | D | IgG2a | 2 |
| 4E6 | >6667 | 2.1 | 29.6 | 19.7 | I | IgG2a | 3 |
| 6H12 | 3.9 | 10.9 | 126.6 | 2206.6 | L | IgG2b | 3 |
| 9H9 | 8.7 | 8.3 | 1144.9 | >6667 | (in progress) | IgG2b | 3 |
| 11D12 | 10.9 | 9.5 | 6385 | >6667 | L | IgG2b | 3 |
| 9G9 | 4.7 | 3.2 | 1698 | 110 | H | IgG2b | 3 |

Epitope Mapping with Mutated VP1

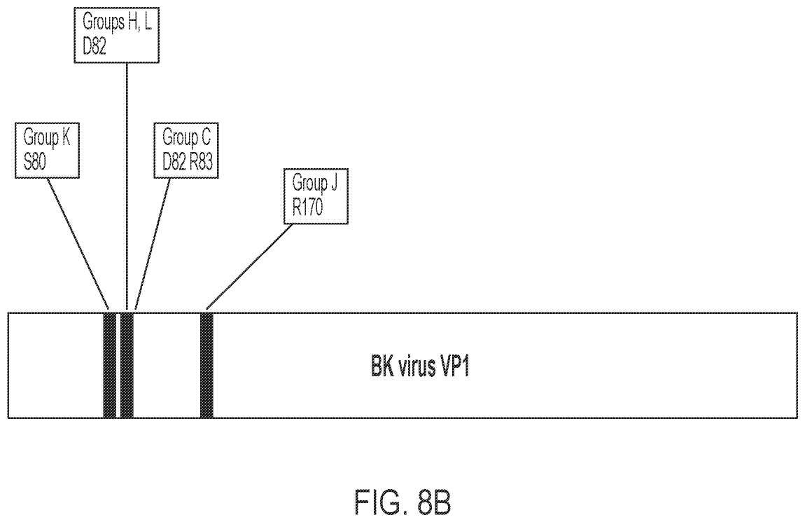

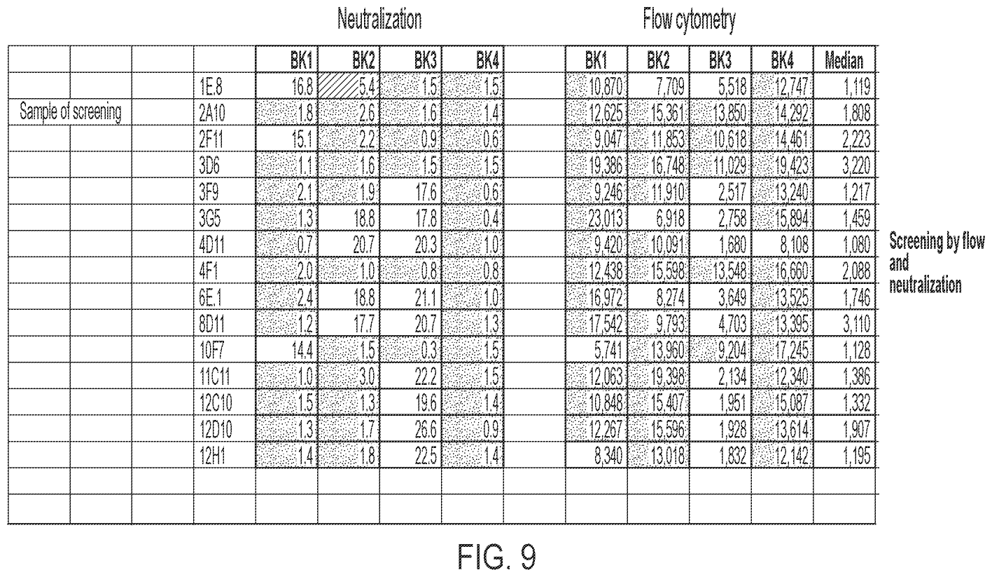

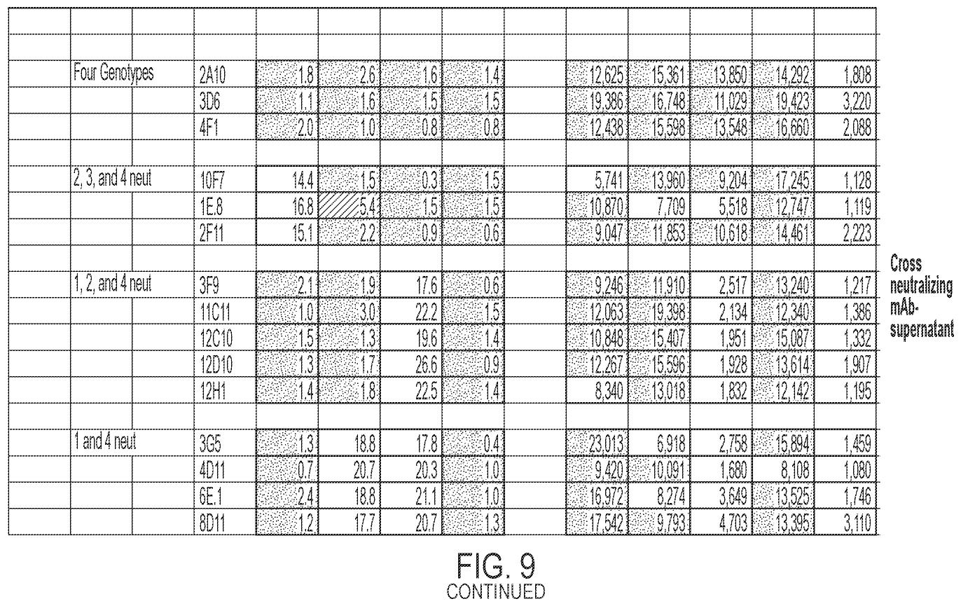

The region of the VP1 believed to be utilized for binding to target cells is between AA 70-85. Some have reported an epitope that utilized aa in the 170 range as well (14). To evaluate epitope specificity, genotype 1 VP1 expression mutant plasmids were constructed. These VP1 expression plasmids has single mutations at positions 80, 82, 83 or 170 or were unmutated (ESD). Cells transfected with these vectors were used in flow cytometry to determine if the inserted mutations altered the binding of the mAb. As shown in FIG. 8A characteristic binding changes can be identified by flow cytometry on cells transfected with wild type VP1 relative to those transfected with the mutant plasmids. The reduction in binding can be observed by all members of the same genetic family but to a different degree indicating key amino acids involved in their binding. Family H is most affected by a change at AA 82 as is family L. Family K is most influenced by a change at position 80 while family J shows profound reduction in binding when R170 is converted to an A.

Complementation of a Cross Reactive Neutralizing Antibody (2B9) with Partial Cross Neutralizing Antibodies from Different Genetic Families.

Monoclonal antibody 2B9, an antibody from family C was not affected significantly by any of the mutational changes. Because of its seeming uniqueness, mixture experiments were set up to determine if its activity could be complemented by antibodies from different families (Table 9). As a baseline, each individual mAb was mixed with an irrelevant isotype matched monoclonal antibody (M2) and dilutions performed. The addition of a partial cross neutralizing antibody from family D (2A10, does not neutralize genotype I) did not improve neutralization with 2B9. However, if 6E1 (only neutralizes I and IV) is mixed with 2A10 (also a partial cross neutralizer) they complement each other to give strong neutralization of all 4 genotypes. 6E1 added to 2B9 improves neutralization against BK1 and BK4 over 2B9 alone showing that they can synergize with each other without interfering. More importantly perhaps is the fact that the combination of 6E1 and 2B9 will simultaneously cover two neutralizing epitopes on BK1 and 4, thus greatly reducing the risk of escape mutations arising from selective antibody pressure.

TABLE 9
Combination of neutralizing antibodies can
complement to improve neutralization

| Gene family | | BK1 | BK2 | BK3 | BK4 |
|---|---|---|---|---|---|
| C | 2B9/M2 | 2.77 | 44 | 5.4 | 10.8 |
| D | 2A10/M2 | >6667 | 55.7 | 6.4 | 5.9 |
| C and D | 2B9/2A10 | 5.5 | 41.4 | 5.1 | 5.8 |
| D | 2A10/M2 | >6667 | 55.7 | 6.4 | 5.9 |
| G | 6E1/M2 | 0.67 | >6667 | >6667 | 49.6 |
| D and G | 2A10/6E1 | 1.1 | 64.4 | 7.3 | 5.8 |
| C | 2B9/M2 | 2.77 | 44 | 5.4 | 10.8 |
| G | 6E1/M2 | 0.67 | >6667 | >6667 | 49.6 |
| C and G | 2B9/6E1 | 0.09 | 53.9 | 7 | 6.8 |

Complementation of Partial Neutralizing Antibodies from Different Genetic Families

Mixtures of partial neutralizers from different families were tested for neutralization of pseudovirus particles from the VP1 of all four genotypes (Table 10). The family L mAb, 6H12, poorly neutralizes BK3 and BK4. However, when mixed with 2A10 from family D which neutralizes BK2, BK3 and BK4 but poorly neutralizes BK1, all four genotypes are strongly neutralized. When 9G9 (poorly neutralizes BK3 and BK4) from family H is mixed with either family D or family E, potent cross neutralization is achieved. Comparable results are observed when mAb from family G and D are mixed. These data argue that the families from which these antibodies derive bind to different but unique sites on the virus allowing them to work cooperatively. If one assumes that each of the individual families binds to a particular epitopic region than 9G9 from family H probably binds to the same site as the most potent cross neutralizing antibody of the family, 15C10. If true, a mixture of 15C10 with either 2A10 or 2F11 should constitute a very potent mixture and may work to block escape mutants from appearing in vivo.

TABLE 9
Combinations of partial neutralizing antibodies can
provide protection against all four genotypes of BK

| Family | Antibodies | BK1 | BK2 | BK3 | BK4 |
|---|---|---|---|---|---|
| L | 6H12/M2 | 0.96 | 2.4 | 172.3 | 944 |
| D | 2A10/M2 | >2000 | 11.2 | 0.654 | 0.72 |
| L & D | 6H12/2A10 | 0.95 | 1.4 | 1.2 | 1.3 |
| H | 9G9/M2 | 2.76 | 2.3 | 1292 | 196 |
| D | 2A10/M2 | >2000 | 11.2 | 0.654 | 0.72 |
| H & D | 9G9/2A10 | 1 | 1.5 | 2.2 | 1.6 |
| H | 9G9/M2 | 2.76 | 2.3 | 1292 | 196 |
| E | 2F11/M2 | >2000 | 148 | 6.1 | 1.85 |
| H & E | 9G9/2F11 | 1.3 | 1.2 | 8.1 | 5 |
| G | 6E1/M2 | 0.78 | >2000 | >2000 | 21 |
| D | 2A10/M2 | >2000 | 11.2 | 0.654 | 0.72 |
| G & D | 6E1/2A10 | 0.43 | 14 | 1.3 | 0.93 |
| Cross | 2B9 | 4.20 | 61.30 | 6.80 | 11.40 |
| neutralizing | 15C10 | 0.1 | 0.58 | 1.86 | 2.30 |
| antibodies | Comparator | 3.90 | 5.50 | 6.30 | 17.00 |

### DISCUSSION

There were 21,167 kidney transplant and 836 kidney-pancreas transplants performed in the US (United States) in 2018 according to the United Network for Organ Sharing. According to the World Health Organization, 70,000 transplants are performed yearly, worldwide. 23000 bone marrow transplants are performed yearly in the US and 50,000 are performed worldwide according to the WHO (World Health Organization). In hematopoietic stem cell transplantation patients, 10-25% develop PyVHC leading to pain, bleeding, obstruction and in some cases, renal failure (6, 7). According to NIDDK, Interstitial cystitis affects 3-8 million women and 1-4 million men every year. BK virus replication occurs in 30-50% of kidney transplant patients with 7-8% leading to graft dysfunction and/or loss (3). BKPyV in blood represents the primary method for diagnosis and levels of BKPyV-DNA (2, 4, 5). BK viremia precedes the development of PyVAN by 1-12 weeks. Blood samples with more than 10⁴ copies per ml indicate presumptive PyVAN. Patients with this level of viremia post transplantation are treated by reduction of immunosuppression and administration of drugs such as leflunomide but the risk of graft rejection escalates (46% loss).

Studies of patients given IVIG support an important role for antibodies in control of BK infection in transplant patients. In the study by Vu et al, patients that failed to respond (persistent viremia>10⁴) were treated with intravenous immunoglobulin (IVIG) (15). Thirty transplant patients with persistent viremia and presumptive PyVAN after standard treatment received IVIG. 27 patients showed clearance of virus from blood and only one of the grafts (from a patient that failed to respond to the IVIG) was lost. Several other studies with IVIG supported the observations and demonstrated control of viremia (16-18). Additionally, animal models of polyoma virus have shown that IgG antibodies can control infection and prevent nephropathy (19).

Further support for the use of antibody therapy as a viable treatment for PyVAN comes from a prospective study of 168 transplant patients by Solis et al (3) the authors showed that high titers of endogenous neutralizing antibody against the strain of BK carried by the donor led to a very significant reduction in risk of viral BK viremia with each log₁₀ rise in antibody titer reducing the risk by 50%. As reported in this and other studies, viral genotype mismatch between the donor and recipient increases the risk of PyVAN significantly (2, 3, 8). These observations support a primary role for antibodies in prevention of disease and argue that monoclonal antibodies may be utilized prophylactically to lessen the risk of PyVAN. To respond to this unmet need CTAD has developed an extensive panel of antibodies that alone or in combination can neutralize all known BK virus genotypes. These antibodies could be utilized prophylactically or therapeutically to reduce virus titers and conceivably protect the donated tissue.

More than 100 mAb were identified and tested against all four BK genotypes. mAb deriving from 4 different genetic families were found that neutralized all four of the BK genotypes.

Recently, a phase II clinical trial to determine the value of BK neutralizing mAb to prevent kidney graft infection had been initiated. The sequence of the monoclonal antibody used in this study was available and it was made genetically. This antibody, referred to as the comparator, was used as a standard to determine the relative efficacy of the antibodies. While the comparator was very good, many of the antibodies showed superior neutralization across the board particularly against genotype IV. The clinical trial was eventually withdrawn due to a reorganization in the company (ClinicalTrials.gov Identifier: NCT03456999).

Clinical trials using antibodies against pathogens like HIV or Ebola utilized pairs of antibodies based upon the assumption that doing this reduced the risk of viral mutants escaping from neutralization of a single antibody. Such antibody “cocktails” are attempted using pairs of antibodies that bind to non-overlapping sites on the viral attachment protein. Since antibodies from a single family use the same genetic elements with slight changes in the CDR or elsewhere resulting from somatic mutation they are likely to bind to an overlapping site on the protein. In order to identify where on the VP1 the antibodies bound, mutants of the VP1 were made at various positions reported to be involved in viral binding. Members of each family were tested by flow cytometry for an effect of the mutations on binding to cells expressing the VP1 following transfection. Family H, which has some of the strongest neutralizers showed reduced binding with a D82E change in the VP1. This change was most apparent with the 9G9 family member which while only a partial cross neutralizer (BK1 and BK2 strongly and BK4 less strong) was profoundly affected by the change. The K family cross neutralizing antibody, 7H8, showed reduced binding when the S80A mutation was incorporated into VP1. Interestingly, the J family, that represents the largest family of neutralizers, was profoundly affected when the R170A mutation was put into the VP1. This mutation would seem to be far from the alleged binding site (AA 70-85), but it has been reported by others that this area of the VP1 is somehow involved in binding of the virus. Lastly, the L family also showed reduced binding with the D82E change in the VP1. The comparator antibody shows reduced binding with either the change at position 82 or 83. Based upon these and other observations studies of complementation were undertaken to determine if mixtures of mAb would show enhanced neutralization. 2B9 when mixed with a partial neutralizer, 2A10 didn't improve but neither did it lose potency. This argues that the two antibodies probably bind to different sites. 2B9 when mixed with 6E1 showed an improvement in neutralization against BK1 and BK4. 2A10 and 6E1, two partial neutralizers did not show enhancement but neither was there any negative impact of mixing the antibodies.

Since it was difficult to show complementation with the potent cross neutralizers, experiments were designed to mix partial cross neutralizers. 6H12(family L) was mixed with 2A10(family D) leading to exceptional cross neutralization not seen with either alone. 6E1 (family G) when mixed with 2A10 showed exceptional activity against all genotypes. 9G9, a member of the H family that includes the potent cross neutralizer, 15C10, when mixed with 2A10 (family D) or 2F11 (family E) showed profound cross neutralizing activity. This data argues that family H members such as 15C10 could be mixed with 2A10 or 2F11 to achieve even more potent neutralization and such a combination is likely to prevent the escape of mutants that may arise in some situations. Further experiments of this type are ongoing.

The primary use for these antibodies would be as a prophylactic treatment given to the recipient prior to kidney transplantation to decrease viral load by preventing dissemination within the host. They should also be effective therapeutically in cases with existing PyVAN. The ideal antibody(s) would be capable of neutralizing all the BK genotypes. Such antibodies would avoid the need to identify genotypes in donor and recipient prior to transplant. While genotype I and IV make up most of the cases, worldwide the hierarchy of the genotypes may vary (2). Moreover, in the kidney transplant study of Solis et al 27% of DNA virus positive patients and 19% of DNA negative patients had neutralizing antibodies to 2 or 3 genotypes that may indicate infections with multiple genotypes are more common than previously believed (3). Ideally, pairs of mAb that could complement one another might be the ideal solution.

### Methods and Materials

### Animals, Cells, Reagents

VelocImmune mice are provided by Regeneron Pharmaceuticals and express human variable light and heavy chains in their antibodies as described (13). HEK-293TT were a gift from Chris Buck, NIH EXPI293F and transfection system were purchased from Invitrogen.

### Immunizations

Expression plasmids for VP1, VP2 and VP3 were used for immunizations with electroporation. Groups of mice were immunized with various protocols and the mice displaying the highest sera titer of neutralizing antibodies against BK I, II, III, and IV pseudoviruses were selected for fusion. The genotypes used for immunizations are shown in Table 7. Boosts with BK pseudovirus preparations were administered IP without adjuvant at 3-4 days prior to fusion (Table 7).

### Flow Cytometry Analysis

EXPI293F cells were transfected with either BK genotype VP1-I, -II, -III, and -IV along with VP2 and VP3 in the ratio of 3:1:1. Forty-eight hrs. later cells were permeabilized, washed and stained with immunized mouse sera (1:200, 1:1000, 1:5000), supernatant from the hybridoma clone or purified mAb in PBS with 0.5% BSA for 1 hr. After 2 washes in PBS/BSA, cells were incubated with Allophycocyanin-conjugated goat anti-mouse IgG secondary (Jackson Immunoresearch) for 30 mins. Cells were washed 1× PBS and resuspended in 50 ul PBS to be analyzed on a HTFC (Intellicyt).

### Pseudovirus Generation

BK pseudoviruses were produced from HEK-293TT cells transfected with plasmids that express VP1, VP2, VP3, and GFP reporter vector at ratio of 2:1:1:1 using Lipofectamine 2000 (Invitrogen). Forty-eight hours after transfection, the cells were harvested at ˜100 million cells/ml in PBS, washed 1× with PBS and lysed by addition of 0.5% Triton X-100, and RNase A/Ti cocktail (Ambion). The lysate was incubated at 37° C. overnight and cell lysates centrifuge at 5,000×g. The pseudovirus particles were isolated by ultracentrifugation through a 27-33-39% iodixanol (Optiprep, Sigma) step gradient at 300,000×g for 3.4 hrs at 16° C. The fractions from the Optiprep column were analyzed for detection of VP1/VP2/VP3 using SDS-PAGE (11, 20)

### Pseudovirus Neutralization Assay:

Neutralization of BK I, II, III, and IV pseudoviruses was performed as follows: 15,000 HEK-293 TT cells were plated in 96 well plates. The next day, hybridoma supernatant (or purified antibody, where indicated) and BK I, II, III, and IV pseudoviruses (titered to 30-60% infectivity) diluted in complete DMEM were pre-incubated at room temperature for 15 inns. 50 ul fresh complete media is added to the cells along with ab/PsV mixture and cultured for 72 hrs. PsV infected cells are visualized by fluorescent microscopy and, after incubation with trypsin and washing, analyzed by flow cytometry using GFP as the readout for infection using the HTFC (Intellicyt).

### ELISA

For measuring antibody binding by ELISA, 96 well plates were coated with 2 ug/ml purified pseudovirus in PBS overnight at 4 degrees C. and blocked for 30 minutes with PBS-0.5% BSA prior to the addition of the antibody containing solutions. Following a 90-minute incubation at room temperature the plates were washed, HRP-linked goat anti-mouse IgG heavy chain specific was added for 60 minutes, washed and substrate added. Plates were read on a microplate reader (BioTek Synergy) at 405 nm 45 minutes later.

### Hybridoma Generation

For the generation of hybridomas, spleens are removed from the boosted animals and fused to SP2/0 myeloma cells. The Sp2/0 cells are grown in Stem Cell media A for three days prior to fusion and mixed with the spleen cells both of which have been washed free of serum using DMEM without serum. The Sp2/0 cells are mixed with spleen cells at a 1:5 ratio and fused by the addition of 1 ml of a 50% solution of PEG 4000 (EMD, Germany). The cell mixture is incubated overnight in Stem Cell Recovery Media (media C), washed, and plated in semisolid selection media, Media D; Stem Cell Technology). Individual clones were isolated 10-12 days later from the plates using the Hamilton/Stem Cell EzPick robot and deposited in 96 well plates containing Stem Cell media E. 48 hours later the supernatants are collected by the robot and deposited in plates for ELISA, flow or neutralization assays.

### Hybridoma Screening

Primary screening was performed using high-throughput flow cytometry on HEK-293 cells transfected with the VP1 from both BKI and BKIV, as detailed above. Briefly, supernatants were added to the cell mixture and following permeabilization antibody binding was identified by the addition of goat anti-mouse IgG (heavy chain specific) APC (Jackson). Positive supernatants were confirmed by a similar flow experiment using HEK-293 cells expressing only one of the four subtypes. In some instances, the supernatants were also transferred to ELISA plates coated with BKI, BKII, BKII or BKIV pseudoviruses at 2 ug/ml. The supernatants (undiluted) were screened for neutralization in a pseudovirus neutralization assay using all four genotypes of the BK virus. It should be noted that a strong but not perfect correlation exists between binding and neutralization.

### Mutagenesis of VP1

Mutagenesis of VP1 was first performed by site directed mutagenesis at four sites located in the predicted neutralization domains of VP1 creating four separate mutants. The sites chosen for mutation were positions that showed some degree of variance among VP1s of all BKs with the specific residue chosen for the mutant representing the other reported residue at that position. These sites included position: 42 (L>Q), 60 (D>N), 73 (E>Q), and 82 (D>E). Site directed mutagenesis was performed following factory supplied protocol using the QuickChange II Site Directed Mutagenesis kit (Agilent Technologies). Briefly, site directed mutagenesis was carried out by designing a forward (5′-3′) and complementary (3′-5′) reverse primer around each site with a single base change that would correspond to the translation of the mutated residue. This changed base would be position roughly in the middle of each primer set. Forward and reverse PCRs initiated from the primers were performed on denatured expression plasmids containing the BK VP1 with conditions that allowed for the single base mismatch. Methylated template stands were digested by DPN1 leaving only newly synthesized plasmid strands containing the incorporated mutation. Newly synthesized plasmid strands were re-annealed and then transformed into competent E. coli for nick repair and amplification. Following experiments assessing binding changes of monoclonal antibodies compared to wildtype VP1, each mutant was ranked based on binding reduction by flow cytometry. Position 82 showed a prominent effect on most of the monoclonals and thus to further elucidate this potential epitope, two more mutants were created that straddled residue 82 by alanine substitution at position 80 (S>A) and 83 (R>A). Further, a sixth mutant at position 170 was also created by alanine substitution (R>A). This position had previously been reported as an important residue in a putative neutralizing epitope located outside the known binding domains of VP1. These last three mutants were generated by de novo DNA synthesis and cloned into pcDNA3.1 mammalian expression vectors (Genscript).

### Isotyping

The isotypes of positive hybridomas were determined using a commercial isotyping kit (BD Pharmingen Cat. 550487). Only IgG antibodies were further tested.

### Quantitation of Monoclonal Antibodies

Mabs were quantitated using label-free, biolayer interferometry (BLI) on an Octet Red96 (ForteBio, Sartorius). Briefly, optically capable biosensors conjugated with either protein A or G were dipped into supernatants containing mabs. Supernatants were measured undiluted and diluted 1:10 in conditioned media and compared to an isotype standard, diluted in conditioned media in a 1:2 dilution series ranging from 100 ug/ml to 1.56 ug/ml. Standard curves were analyzed in the ForteBio Data Analysis Software v.10 (ForteBio) using a 5-parameter logistics (5PL) dose response curve fitting model on the initial binding slopes. Mab concentrations are then calculated from the standard curve. Diluted samples were compared to undiluted samples after application of the respective dilution factors. Total concentrations were averaged together from the diluted and undiluted sample.

### Purification of Monoclonal Antibodies

Hybridomas producing antibodies positive for BK virus VP1 were grown in media E from 2 days and transferred to Serum Free Hybridoma Media (Gibco, ThermoFisher). The serum free supernatants from the hybridomas are purified using FPLC on a Akta Chromatography System (GE) using Protein A or Protein G HiTrap columns (GE).

### REFERENCES

1. Stolt A, Sasnauskas K, Koskela P, Lehtinen M, Dillner J. 2003. Seroepidemiology of the human polyomaviruses. J Gen Virol 84:1499-1504.
2. Hirsch H H, Randhawa P S, Practice ASTIDCo. 2019. BK polyomavirus in solid organ transplantation-Guidelines from the American Society of Transplantation Infectious Diseases Community of Practice. Clin Transplant 33:e13528.
3. Solis M, Velay A, Porcher R, Domingo-Calap P, Soulier E, Joly M, Meddeb M, Kack-Kack W, Moulin B, Bahram S, Stoll-Keller F, Barth H, Caillard S, Fafi-Kremer S. 2018. Neutralizing Antibody-Mediated Response and Risk of BK Virus-Associated Nephropathy. J Am Soc Nephrol 29:326-334.
4. Helantera I, Salmela K, Kyllonen L, Raisanen-Sokolowski A, Auvinen E, Mannonen L, Koskinen P, Lautenschlager I. 2012. BK virus viremia in a well-HLA-matched kidney transplant population mainly on low-dose cyclosporine-based immunosuppression. Clin Transplant 26:E596-601.
5. Schaub S, Hirsch H H, Dickenmann M, Steiger J, Mihatsch M J, Hopfer H, Mayr M. 2010. Reducing immunosuppression preserves allograft function in presumptive and definitive polyomavirus-associated nephropathy. Am J Transplant 10:2615-23.
6. Bennett S M, Broekema N M, Imperiale M J. 2012. BK polyomavirus: emerging pathogen. Microbes Infect 14:672-83.
7. Gander R, Asensio M, Guillen G, Royo G F, Bolanos A, Perez M, Diaz-De-Heredia C, Benitez M, Lopez M. 2018. Hemorrhagic cystitis after hematopoietic stem cell transplantation: A challenge for the pediatric urologist. J Pediatr Urol 14:366-373.
8. Abend J R, Changala M, Sathe A, Casey F, Kistler A, Chandran S, Howard A, Wojciechowski D. 2017. Correlation of BK Virus Neutralizing Serostatus With the Incidence of BK Viremia in Kidney Transplant Recipients. Transplantation 101:1495-1505.
9. Ali A M, Gibson I W, Birk P, Blydt-Hansen T D. 2011. Pretransplant serologic testing to identify the risk of polyoma BK viremia in pediatric kidney transplant recipients. Pediatr Transplant 15:827-34.
10. Sood P, Senanayake S, Sujeet K, Medipalli R, Van-Why S K, Cronin D C, Johnson C P, Hariharan S. 2013. Donor and recipient BKV-specific IgG antibody and posttransplantation BKV infection: a prospective single-center study. Transplantation 95:896-902.
11. Pastrana D V, Brennan D C, Cuburu N, Storch G A, Viscidi R P, Randhawa P S, Buck C B. 2012. Neutralization serotyping of BK polyomavirus infection in kidney transplant recipients. PLoS Pathog 8:e1002650.
12. Seifert M E, Gunasekaran M, Horwedel T A, Daloul R, Storch G A, Mohanakumar T, Brennan D C. 2017. Polyomavirus Reactivation and Immune Responses to Kidney-Specific Self-Antigens in Transplantation. J Am Soc Nephrol 28:1314-1325.
13. Murphy A J, Macdonald L E, Stevens S, Karow M, Dore A T, Pobursky K, Huang T T, Poueymirou W T, Esau L, Meola M, Mikulka W, Krueger P, Fairhurst J, Valenzuela D M, Papadopoulos N, Yancopoulos G D. 2014. Mice with megabase humanization of their immunoglobulin genes generate antibodies as efficiently as normal mice. Proc Natl Acad Sci USA 111:5153-8.
14. Abend J, Dragic Z, Feire A L, Knapp M, Kovacs S, Traggiai E, Wang L, Wang Y, Wu D, Xu F. 2017. Polyomavirus neutralizing antibodies WO 2017/046676 A1.
15. Vu D, Shah T, Ansari J, Naraghi R, Min D. 2015. Efficacy of intravenous immunoglobulin in the treatment of persistent BK viremia and BK virus nephropathy in renal transplant recipients. Transplant Proc 47:394-8.
16. Kable K, Davies C D, O'Connell P J, Chapman J R, Nankivell B J. 2017. Clearance of BK Virus Nephropathy by Combination Antiviral Therapy With Intravenous Immunoglobulin. Transplant Direct 3:e142.
17. Sener A, House A A, Jevnikar A M, Boudville N, McAlister V C, Muirhead N, Rehman F, Luke P P. 2006. Intravenous immunoglobulin as a treatment for BK virus associated nephropathy: one-year follow-up of renal allograft recipients. Transplantation 81:117-20.
18. Sharma A P, Moussa M, Casier S, Rehman F, Filler G, Grimmer J. 2009. Intravenous immunoglobulin as rescue therapy for BK virus nephropathy. Pediatr Transplant 13:123-9.
19. Nickeleit V, Singh H K, Rivier L H. 2018. Antibodies Can Extenuate Polyomavirus Infections. J Am Soc Nephrol 29:1577.
20. Buck C B, Thompson C D. 2007. Production of papillomavirus-based gene transfer vectors. Curr Protoc Cell Biol Chapter 26:Unit 26 1.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

1. (canceled)

2. (canceled)

3. An isolated antibody or antigen binding fragment thereof comprising a heavy chain variable region (VH) having three complementarity determining regions (CDRs) of HCDR1, HCDR2, and HCDR3, and a light chain variable region (VL) having three CDRs of LCDR1, LCDR2, and LCDR3, wherein:
(a) HCDR1 comprises SEQ ID NO: 113,
HCDR2 comprises SEQ ID NO: 114,
HCDR3 comprises SEQ ID NO: 115,
LCDR1 comprises SEQ ID NO: 116,
LCDR2 comprises SEQ ID NO: 117, and
LCDR3 comprises SEQ ID NO: 118,
(b) HCDR1 comprises SEQ ID NO: 97,
HCDR2 comprises SEQ ID NO: 98,
HCDR3 comprises SEQ ID NO: 99, 131, or 139,
LCDR1 comprises SEQ ID NO: 100,
LCDR2 comprises SEQ ID NO: 101, and
LCDR3 comprises SEQ ID NO: 102;
(c) HCDR1 comprises SEQ ID NO: 1,
HCDR2 comprises SEQ ID NO: 2,
HCDR3 comprises SEQ ID NO: 3,
LCDR1 comprises SEQ ID NO: 4,
LCDR2 comprises SEQ ID NO: 5, and
LCDR3 comprises SEQ ID NO: 6;
(d) HCDR1 comprises SEQ ID NO: 9,
HCDR2 comprises SEQ ID NO: 10,
HCDR3 comprises SEQ ID NO: 11,
LCDR1 comprises SEQ ID NO: 12,
LCDR2 comprises SEQ ID NO: 13, and
LCDR3 comprises SEQ ID NO: 14;
(e) HCDR1 comprises SEQ ID NO: 17,
HCDR2 comprises SEQ ID NO: 18,
HCDR3 comprises SEQ ID NO: 19,
LCDR1 comprises SEQ ID NO: 20,
LCDR2 comprises SEQ ID NO: 21, and
LCDR3 comprises SEQ ID NO: 22;
(f) HCDR1 comprises SEQ ID NO: 25,
HCDR2 comprises SEQ ID NO: 26,
HCDR3 comprises SEQ ID NO: 27,
LCDR1 comprises SEQ ID NO: 28,
LCDR2 comprises SEQ ID NO: 29, and
LCDR3 comprises SEQ ID NO: 30;
(g) HCDR1 comprises SEQ ID NO: 33,
HCDR2 comprises SEQ ID NO: 34,
HCDR3 comprises SEQ ID NO: 35,
LCDR1 comprises SEQ ID NO: 36,
LCDR2 comprises SEQ ID NO: 37, and
LCDR3 comprises SEQ ID NO: 38;
(h) HCDR1 comprises SEQ ID NO: 41,
HCDR2 comprises SEQ ID NO: 42,
HCDR3 comprises SEQ ID NO: 43,
LCDR1 comprises SEQ ID NO: 44,
LCDR2 comprises SEQ ID NO: 45, and
LCDR3 comprises SEQ ID NO: 46;
(i) HCDR1 comprises SEQ ID NO: 49,
HCDR2 comprises SEQ ID NO: 50,
HCDR3 comprises SEQ ID NO: 51,
LCDR1 comprises SEQ ID NO: 52,
LCDR2 comprises SEQ ID NO: 53, and
LCDR3 comprises SEQ ID NO: 54;
(j) HCDR1 comprises SEQ ID NO: 65,
HCDR2 comprises SEQ ID NO: 66,
HCDR3 comprises SEQ ID NO: 67,
LCDR1 comprises SEQ ID NO: 68,
LCDR2 comprises SEQ ID NO: 69, and
LCDR3 comprises SEQ ID NO: 70;
(k) HCDR1 comprises SEQ ID NO: 73,
HCDR2 comprises SEQ ID NO: 74,
HCDR3 comprises SEQ ID NO: 75,
LCDR1 comprises SEQ ID NO: 76,
LCDR2 comprises SEQ ID NO: 77, and
LCDR3 comprises SEQ ID NO: 79;
(l) HCDR1 comprises SEQ ID NO: 81,
HCDR2 comprises SEQ ID NO: 82,
HCDR3 comprises SEQ ID NO: 83,
LCDR1 comprises SEQ ID NO: 84,
LCDR2 comprises SEQ ID NO: 85, and
LCDR3 comprises SEQ ID NO: 86;
(m) HCDR1 comprises SEQ ID NO: 89,
HCDR2 comprises SEQ ID NO: 90,
HCDR3 comprises SEQ ID NO: 91,
LCDR1 comprises SEQ ID NO: 92,
LCDR2 comprises SEQ ID NO: 93, and
LCDR3 comprises SEQ ID NO: 94;
(n) HCDR1 comprises SEQ ID NO: 105,
HCDR2 comprises SEQ ID NO: 106,
HCDR3 comprises SEQ ID NO: 107,
LCDR1 comprises SEQ ID NO: 108,
LCDR2 comprises SEQ ID NO: 120, and
LCDR3 comprises SEQ ID NO: 110;
(o) HCDR1 comprises SEQ ID NO: 121,
HCDR2 comprises SEQ ID NO: 122,
HCDR3 comprises SEQ ID NO: 123,
LCDR1 comprises SEQ ID NO: 124,
LCDR2 comprises SEQ ID NO: 125, and
LCDR3 comprises SEQ ID NO: 126;
(p) HCDR1 comprises SEQ ID NO: 145,
HCDR2 comprises SEQ ID NO: 146,
HCDR3 comprises SEQ ID NO: 147,
LCDR1 comprises SEQ ID NO: 148,
LCDR2 comprises SEQ ID NO: 149, and
LCDR3 comprises SEQ ID NO: 150;
(q) HCDR1 comprises SEQ ID NO: 153,
HCDR2 comprises SEQ ID NO: 154,
HCDR3 comprises SEQ ID NO: 155,
LCDR1 comprises SEQ ID NO: 156,
LCDR2 comprises SEQ ID NO: 157, and
LCDR3 comprises SEQ ID NO: 158;
(r) HCDR1 comprises SEQ ID NO: 161,
HCDR2 comprises SEQ ID NO: 162,
HCDR3 comprises SEQ ID NO: 163,
LCDR1 comprises SEQ ID NO: 164,
LCDR2 comprises SEQ ID NO: 165, and
LCDR3 comprises SEQ ID NO: 166;
(s) HCDR1 comprises SEQ ID NO: 169,
HCDR2 comprises SEQ ID NO: 170,
HCDR3 comprises SEQ ID NO: 171,
LCDR1 comprises SEQ ID NO: 172,
LCDR2 comprises SEQ ID NO: 173, and
LCDR3 comprises SEQ ID NO: 174; or
(t) HCDR1 comprises SEQ ID NO: 177,
HCDR2 comprises SEQ ID NO: 178,
HCDR3 comprises SEQ ID NO: 179,
LCDR1 comprises SEQ ID NO: 180,
LCDR2 comprises SEQ ID NO: 181, and
LCDR3 comprises SEQ ID NO: 182.

4. (canceled)

5. (canceled)

6. The isolated antibody or antigen binding fragment thereof of claim 3, wherein:
(a) the VH comprises at least 90% sequence identity to any one of SEQ ID NO: 7, 15, 23, 31, 39, 47, 55, 63, 71, 79, 87, 95, 103, 111, 119, 127, 135, 143, 151, 159, 167, 175, or 183; and
(b) the VL comprises at least 90% sequence identity to any one of SEQ ID NO: 8, 16, 24, 32, 40, 48, 56, 64, 72, 80, 88, 96, 104, 112, 120, 128, 136, 144, 152, 160,168, 176, or 184.

7. (canceled)

8. The isolated antibody or antigen binding fragment thereof of claim 6, wherein,
(a) VH comprises SEQ ID NO: 119, and VL comprises SEQ ID NO: 120;
(b) VH comprises SEQ ID NO: 103, and VL comprises SEQ ID NO: 104;
(c) VH comprises SEQ ID NO: 135, and VL comprises SEQ ID NO: 136;
(d) VH comprises SEQ ID NO: 143, and VL comprises SEQ ID NO: 144;
(e) VH comprises SEQ ID NO: 7, and VL comprises SEQ ID NO: 8;
(f) VH comprises SEQ ID NO: 15, and VL comprises SEQ ID NO: 16;
(g) VH comprises SEQ ID NO: 31, and VL comprises SEQ ID NO: 32;
(h) VH comprises SEQ ID NO: 39, and VL comprises SEQ ID NO: 40;
(i) VH comprises SEQ ID NO: 47, and VL comprises SEQ ID NO: 48;
(j) VH comprises SEQ ID NO: 55, and VL comprises SEQ ID NO: 56;
(k) VH comprises SEQ ID NO: 63, and VL comprises SEQ ID NO: 64;
(l) VH comprises SEQ ID NO: 71, and VL comprises SEQ ID NO: 72;
(m) VH comprises SEQ ID NO: 79, and VL comprises SEQ ID NO: 80;
(n) VH comprises SEQ ID NO: 87, and VL comprises SEQ ID NO: 88;
(o) VH comprises SEQ ID NO: 95, and VL comprises SEQ ID NO: 96;
(p) VH comprises SEQ ID NO: 111, and VL comprises SEQ ID NO: 112;
(p) VH comprises SEQ ID NO: 127, and VL comprises SEQ ID NO: 128;
(r) VH comprises SEQ ID NO: 151, and VL comprises SEQ ID NO: 152;
(s) VH comprises SEQ ID NO: 159, and VL comprises SEQ ID NO: 160;
(t) VH comprises SEQ TD NO: 167, and VL comprises SEQ TD NO: 168;
(u) VH comprises SEQ TD NO: 175, and VL comprises SEQ TD NO: 176; or
(v) VH comprises SEQ TD NO: 183, and VL comprises SEQ TD NO: 184.

9.-18. (canceled)

19. The antibody or antigen binding fragment thereof of claim 3, wherein the VH of the antibody or antigen binding fragment thereof comprises heavy chain framework regions HFR1, HFR2, HFR3, HFR4, and wherein the VL of the antibody or antigen binding fragment thereof comprises light chain framework regions LFR1, LFR2, LFR3, LFR4, wherein:
(a) the HFR1 region comprises the amino acid sequence of any one of SEQ ID NOs: 185, 193, 197, 205, 213, 226, 234, 241, 250, or 269;
(b) the HFR2 region comprises the amino acid sequence of any one of SEQ ID NOs: 186, 194, 198, 206, 214, 223, 227, 232, 242, or 253;
(c) the HFR3 region comprises the amino acid sequence of any one of SEQ ID NOs: 187, 195, 199, 207, 215, 219, 224, 228, 233, 235, 243, 251, 254, 258, 259, 262, 265, 267, or 270;
(d) the HFR4 region comprises the amino acid sequence of any one of SEQ ID NOs: 188, 200, 208, 220, 229, 236, 244, or 271;
(e) the LFR1 region comprises the amino acid sequence of any one of SEQ ID NOs: 189, 201, 209, 237, 245, 255, or 260;
(f) the LFR2 region comprises the amino acid sequence of any one of SEQ ID NOs: 190, 202, 210, 216, 221, 225, 230, 238, 246, 261, 263, or 272;
(g) the LFR3 region comprises the amino acid sequence of any one of SEQ ID NOs: 191, 196, 203, 211, 217, 222, 230, 239, 247, 249, 252, 256, 264, 266, 268, or 273; and
(h) the LFR4 region comprises the amino acid sequence of any one of SEQ ID NOs: 192, 204, 212, 218, 240, 248, or 257.

20. The antibody or antigen binding fragment thereof of claim 3, wherein the antibody or antigen binding fragment thereof further comprises a heavy chain constant (CH) domain and a light chain constant (CL) domain, wherein:
(a) said CH domain is selected from the group consisting of IgG, IgG1, IgG2, IgG2a, IgG2b, IgG2c, IgG3, IgG4, IgA, IgA1, IgA2, IgD, IgM, and IgE constant domains, and comprises a sequence with least 90% sequence identity to one of SEQ ID NOs: 274-290; and
(b) said CL domain is selected from the group consisting of 1 g kappa and Ig lambda constant domains, and comprises a sequence with at least 90% sequence identity to one of SEQ ID NOs: 291-301.

21. The antibody or antigen binding fragment thereof of claim 3, wherein the antibody or antigen binding fragment thereof is a monoclonal antibody, a chimeric antibody, a humanized antibody, a human engineered antibody, a human antibody, a single chain antibody (scFv), or an antibody fragment.

22. The antibody or antigen binding fragment thereof of claim 3, wherein the antibody or antigen binding fragment thereof has reduced glycosylation, no glycosylation, or is hypofucosylated.

23. A pharmaceutical composition comprising the antibody or antigen binding fragment thereof of claim 3 and a pharmaceutically acceptable excipient.

24.-28. (canceled)

29. A method of neutralizing a BK virus infection in a subject in need thereof, the method comprising administering an effective amount of the antibody or antigen binding fragment thereof of claim 3 to the subject.

30. The method of claim 29, wherein the subject is diagnosed with BK virus infection or BK viremia.

31. A method of treating or reducing the likelihood of a BK virus or JC virus associated disorder, the method comprising administering to a subject in need thereof an effective amount of the antibody or antigen binding fragment thereof, wherein the BK virus or JC virus associated disorder is selected from the group consisting of transplant rejection, graft-versus-host disease (GvHD), nephropathy, BKVAN, hemorrhagic cystitis (HC), Progressive Multifocal Leukoencephalopathy (PML), granule cell neuronopathy (GCN), interstitial kidney disease, ureteral stenosis, vasculitis, colitis, retinitis, meningitis, immune reconstitution inflammatory syndrome (IRIS).

32. The method of claim 31, wherein the antibody or antigen binding fragment thereof is administered via injection or infusion.

33. (canceled)

34. The method of claim 29, method further comprising an additional therapeutic agent, wherein the therapeutic agent is an immunosuppressive agent.

35. The method of claim 34, wherein the immunosuppressive agent is a monophosphate dehydrogenase inhibitor, a purine synthesis inhibitor, a calcineurin inhibitor, or an mTOR inhibitor.

36. The method of claim 34, wherein the immunosuppressive agent is mycophenolate mofetil (MMF), mycophenolate sodium, azathioprine, tacrolimus, sirolimus, or cyclosporine.

37. The method of claim 31, wherein the therapeutic agent is an additional anti-VPI antibody.

38. A nucleic acid that encodes the antibody or antigen binding fragment of claim 3.

39. A vector comprising the nucleic acid of claim 38.

40. An isolated host cell comprising the vector of claim 39.

41.-43. (canceled)

44. A method of reducing the risk of transplant rejection in a transplant recipient receiving a donor organ, comprising
(i) determining serotype of BK virus present in the donor organ,
(ii) selecting an antibody or antigen binding fragment thereof of claim 3, wherein the antibody or antigen binding fragment thereof neutralizes the serotype of BK virus present in the donor organ, and
(iii) administering the selected antibody or antigen binding fragment thereof to the transplant recipient via injection or infusion.
